(19) United States
(12) Patent Application Publication
(10) Pub. No.: US 2024/0000835 A1
(43) Pub. Date: Jan. 4, 2024

(54) NUCLEIC ACID CONSTRUCTS AND CELLS

(71) Applicant: Cancer Research Technology Limited, London (GB)

(72) Inventors: Francis MUSSAI, Birmingham (GB); Carmela De Santo, Birmingham (GB)

(21) Appl. No.: 18/038,581

(22) PCT Filed: Nov. 24, 2021
(86) PCT No.: PCT/GB2021/053057

§ 371 Date: May 24, 2023

(30) Foreign Application Priority Data
Nov. 25, 2020 (GB) 2018554.2

(51) Int. Cl. A61K 35/17; C12N 5/0783; C07K 14/435; C07K 16/30; C07K 16/28; A61P 35/00; A61K 39/00

(52) U.S. Cl. CPC A61K 35/17; C12N 5/0636; C12N 5/0646; C07K 14/435; C07K 16/3084; C07K 16/2803; A61P 35/00; A61K 39/4631; A61K 39/4613; A61K 39/4611; A61K 39/464411; C07K 16/2863; A61K 39/464404; A61K 39/464471; A61K 2239/28; A61K 2239/13

(57) ABSTRACT

The invention relates to nucleic acid constructs comprising a combination of a first sequence encoding a CAR and second sequence encoding an amino acid transporter, and to cells comprising such nucleic acid constructs. It also relates to methods of manufacturing said cells, and to pharmaceutical compositions comprising said nucleic acid constructs or cells, for use in the treatment of diseases with cellular amino-acid depletion such as cancer.

### FIELD OF THE INVENTION

The invention relates to nucleic acid constructs, and to cells comprising such nucleic acid constructs. It also relates to methods of manufacturing therapeutically modified cells, and to pharmaceutical compositions comprising nucleic acid constructs or cells.

### INTRODUCTION

Fusion proteins with target-binding capabilities have been used in a number of therapeutic applications. Most notably, T cells engineered to express chimeric antigen receptors (CARs) have been used in the treatment of cancer. However, as discussed further below, despite showing considerable clinical promise, such treatments have not been universally effective.

### CAR-T Failure in Pre-Clinical and Clinical Studies

Despite advances in cytotoxic chemotherapy for both adult and paediatric cancers, it is clear that a number of major cancer subtypes still have an extremely poor prognosis. Immune therapies provide an alternative approach to targeting the malignant cancer cells directly, and avoid the toxic side-effects to normal cells of standard approaches.

Chimeric Antigen Receptor (CAR)-T cells (CAR-T) are autologous patient-derived T cells which have been engineered, typically with an antibody fragment (scFv), to specifically recognise surface antigens on tumour cells. The proof-of-principle of using CAR-T cells to successfully treat paediatric cancers has been established in patients with chemo-resistant, relapsed paediatric B Acute Lymphoblastic Leukaemia who underwent rapid and sustained remissions using anti-CD19 CAR T cells. In solid tumours neuroblastoma, the most common solid cancer of childhood, has been the model of choice and proved highly informative in the response of solid tumours to CAR-T cell therapy. Preclinical studies indicate that CAR T cells that recognise disialoganglioside 2 (GD2) antigen could represent a powerful new way of killing neuroblastoma cells. Although neuroblastoma has become the paradigm for CAR-T cell development against solid tumours, only limited anti-tumour efficacy has been seen in preclinical models and early phase trials. First generation anti-GD2 CAR T cells failed to persist in vivo and had minimal anti-tumour effects. Second generation anti-GD2-CAR T cells (with CD28 or 4-1BB costimulatory domains) had improved persistence in vivo, leading to moderate tumour regressions, but become functionally exhausted in the presence of neuroblastoma. In humans, a study of anti-GD2 CAR T cells made the key observation that despite infusion of large numbers of these cells, CAR T cell numbers become low or undetectable within weeks, and that the majority of patients with active disease did not achieve a complete remission. Importantly patients who had low-level persistence of CAR T cells had a longer survival. These findings suggest that the local and systemic tumour microenvironment impairs persistence of CAR-T cells, despite the presence of large target antigenic load on residual neuroblastoma tumours.

CAR-T cell therapy has also been tested against a limited number of other solid tumours in vitro, in vivo, and in man. In each case results against these malignancies have failed to replicate the exciting data found for anti-CD19 CAR-T cells in ALL.

### Acute Myeloid Leukaemia

Acute Myeloid Leukaemia, is the most common acute leukaemia of adults and the second most common leukaemia of childhood. Incidence increases with age, and for patients with high risk or relapsed disease the prognosis is extremely poor with survival <12 months in adults, despite haematopoietic stem cell transplant. For elderly patients or those with co-existing morbidities standard chemotherapeutic regimens are poorly tolerated leading to sub-optimal treatment, and an in ability to achieve cure. Few effective new drugs have been developed for AML, as such immunotherapeutics offers the potential of a different approach. CD33 is almost universally expressed on AML blasts and has proved to be an effective target for immunotoxin-based therapeutics (Gemtuzumab ozogamicin). Anti-CD33 CAR-T cells are cytotoxic to AML blasts in vitro and eradicate leukaemic burden in vivo. On this basis a Phase I clinical trial of anti-CD33 CAR T cells has been initiated in China (NCT01864902 and NCT02958397). Reports from 1 patient with chemo-refractory AML showed a reduction in bone marrow AML blasts. These results provide proof-of principle that anti-CD33 CAR T cells can be effective. However disease relapsed by 9 weeks post CAR infusion despite measurable CAR-T cells remaining in both the blood and bone marrow. The finding suggests that the CAR-T cells have been rendered inactive, by the leukaemia microenvironment (no evidence for CD33 loss on AML blasts as a mechanism of escape).

### Mesothelioma, Ovarian and Pancreatic Cancer

Mesothelioma, an asbestos related tumour with almost universally poor prognosis in adults, expresses the cell surface glycoprotein mesothelin. Mesothelin is also expressed on epithelial cancers, such as ovarian, lung adenocarcinoma, and pancreatic cancer. Mesothelin has been demonstrated to be an effective and selective target for passive immunotherapy with immunotoxins such as SS1P leading to its choice for development in CAR T technologies. In murine models anti-mesothelin CAR-T cells demonstrate clear and persistent anti-tumour activity. Anti-mesothelin CAR-T cells have also been administered to patients with these tumours and although limited responses were detected (PR, SD) in each case the tumours progressed. CAR-T cell persistence was extremely poor with cells becoming undetectable within only days of initial or repeat administration. Even when CAR-T cells are placed within the tumour, and hence in close proximity to target antigen, responses remain muted suggesting a strong immunosuppressive microenvironment that reduces the function of the T cells.

### Glioblastoma

Glioblastoma is one of the most devastating brain tumours of both adults and children, with patients frequently experiencing a rapid disease progression and treatment failure despite intensive chemotherapy and radiotherapy based regimens. Glioblastomas express a variant of the Epidermal Growth Factor Receptor—EGFRvIII, providing a tumour-specific antigen which can be targeted by immunotherapy. EGFRvIII may also be expressed on approximately one third of advanced colorectal cancers. Anti-EGFRvIIII CAR-T cells demonstrated disease control of glioblastomas in orthotopic murine xenografts. However in all cases tumours continue to grow, leading to murine death, despite detectable levels of CAR-T cells in all organs including the brain. Again this data suggests that the CAR-T cells are inactivated by the tumour microenvironment. A Phase I trial based on this rationale is currently underway (NCT02844062, NCT02664363).

### Arginine and the Immunosuppressive Microenvironment

Arginine is a semi-essential amino acid, required by healthy tissues for a number of cell processes including cell viability, proliferation and protein synthesis. Whole body arginine levels are maintained principally through dietary intake, and to a lesser extent by synthesis from precursors in an ‘intestinal-renal axis’. At a cellular level, arginine is imported from the extracellular fluid via Cationic Amino Acid (CAT; SLC7A) family of transporters and enters the urea cycle. In conditions of high demand such as inflammation, pregnancy, and cancer, arginine levels can become limited in the local tissue microenvironment and systemically. Some tissues and cells can protect themselves by resynthesizing arginine from precursors, through the expression of ArgininoSuccinate Synthase (ASS1) and Ornithine Transcarbamylase (OTC). Cells which lack expression of at least one of these enzymes are dependent on import of extracellular arginine, a state known as arginine auxotrophism.

Previous studies have suggested that inhibition of arginase at the tumour site may be beneficial in addressing the issues of poor CAR T cell activity in vivo.

### SUMMARY OF THE INVENTION

In a first aspect of the invention there is provided a nucleic acid construct comprising a first sequence encoding an amino acid transporter, and a second sequence encoding a fusion target-binding protein. These may also be referred to as “constructs of the invention” for the sake of brevity. The references to “first” and “second” sequences are simply made to allow distinction between the relevant sequence. They should not be taken as imposing any limitation on the order of these sequences within the construct, unless the context requires otherwise. The fusion target-binding protein may be a chimeric antibody receptor (CAR), as considered in more detail below. The first aspect of the invention also provides polypeptides that are the expression product of a nucleic acid construct in accordance with the first aspect of the invention. Such polypeptides may comprise constituents as described with respect to those encoded by the nucleic acid constructs defined herein.

According to a second aspect of the invention there is provided a cell comprising a nucleic acid construct according to any preceding claim. The cell may be selected from the group consisting of: a T cell; and a natural killer (NK) cell. The nucleic acid construct may be stably incorporated in the genome of a cell of the invention. The second aspect of the invention also provides a cell comprising a polypeptide that is the expression product of a nucleic acid construct in accordance with the first aspect of the invention.

A cell or nucleic acid construct of the invention may be used as a medicament in the prevention and/or treatment of a disease. Suitably the disease to be prevented and/or treated is cancer.

In a third aspect of the invention there is provided a method of manufacturing a therapeutically modified cell, the method comprising providing a cell with a nucleic acid sequence encoding a fusion target-binding protein and a nucleic acid sequence encoding an amino acid transporter such that the nucleic acid sequences are expressed by the cell to produce an amino acid transporter and a fusion target-binding protein. Suitably the required nucleic acid sequences may be provided by means of a nucleic acid construct in accordance with the first aspect of the invention.

In a fourth aspect of the invention there is provided a pharmaceutical composition comprising a nucleic acid construct of the first aspect of the invention, or a cell of the second aspect of the invention, and a pharmaceutically acceptable carrier or diluent.

It will be appreciated that, except where the context requires otherwise, embodiments of the invention described in the context of nucleic acid constructs of the invention are also applicable to cells, methods and compositions of the invention, and vice versa.

### BRIEF DESCRIPTION OF THE DRAWING

FIG. 1 shows SLC7A5 or SLC7A11 modified CAR-T cells can be manufactured. Panel a shows Flow cytometry demonstrating successful transduction (CD34+) of human T cells with the anti-CD33 CAR and SLC7A5 or SLC7A11. Representative staining of 2 donors. Panel b shows CAR-T cells can be sorted to a high degree of purity based on CD34 isolation. Panels c and d show that Flow cytometry of modified CAR-T cells demonstrated no negative impact on naïve (CD45RA+CD62L+):effector(CD45RA+CD62L-) phenotype. Results demonstrating that cells of the invention can be manufactured, and purified (panels “a” and “b”), and that transduction of T cells with nucleic acid constructs of the invention does not have a negative impact upon levels of naïve or effector T cell phenotypes.

FIG. 2 shows enhanced proliferation of SLC7A5 or SLC7A11 modified CAR-T cells. Panel a shows Flow cytometry of modified CAR-T cells demonstrated no negative impact on immune checkpoint exhaustion marker expression or panel b shows capacity to recognise and kill target tumour cells. Panel c shows SLC7A5 or SLC7A11 modified CAR-T cells demonstrate enhanced proliferation in the presence of tumour cells (THP-1). The results demonstrate that transduction of T cells with nucleic acid constructs of the invention, to yield cells of the invention, does not adversely impact cell exhaustion markers (panel “a”), or the cytotoxic activity of the cells (panel “b”). Furthermore, the cells of the invention demonstrate a marked increase in proliferation in response to contact with cancer cells expressing the target protein recognised by the fusion target-binding protein.

FIG. 3 shows panel “a”: Flow cytometry prior to cell sorting demonstrating expression of CAR-T constructs in human T cells, using lentiviral vectors. Panel “b” shows Western blotting analysis of amino acid transporter expression by cells of the invention. Panel “c” shows SLC7A11 modified CAR-T cells demonstrate increased uptake of fluorescently labelled Cysteine Probe as measured by flow cytometry. Panel “d” shows SLC7A11(xCT) modified CAR-T cells demonstrate increased release of IFN-gamma in the presence of THP-1 tumour target cells, consistent with enhanced activation under low cysteine conditions (75% cysteine free) compared to unmodified and MOCK CAR-T cells. No activation is seen in the absence of tumour target cells. The results demonstrate that cells of the invention can be manufactured using lentiviral vectors comprising a nucleic acid sequence of the invention (panel “a”), that cells of the invention express elevated levels of amino acid transporters (panel “b”), and have increased amino acid uptake (panel “c”) and that they demonstrate increased activation in response to the presence of cancer cells in amino acid depleted conditions.

FIG. 4 shows in panel “a” the percentage of viable control CAR T cells remaining after culture in tryptophan or cysteine-depleted medium. Panel “b” shows proliferation of healthy donor T cells (anti-CD3/CD28 antibody stimulated) is inhibited in leucine- or tryptophan- or cysteine-depleted media after 72 hours, measured by 3 H-thymidine assay. Panel “c” shows CAR T cells were labelled with CFSE, seeded in CD3/CD28 coated wells, and cultured for 96 hours in medium containing 10% dialysed FBS and supplemented with titrated concentrations of tryptophan. The data show the % of proliferating CAR+T cells after 96hours. Data show mean of duplicate cultures+SD. The data show that viability of control CAR T cells is reduced in amino acid depleted conditions (panel “a”), that proliferation of stimulated healthy donor T cells is inhibited by depletion of leucine, tryptophan or cysteine (panel “b”), and that proliferation of stimulated control CAR T cells is reduced in proportion to a reduction in tryptophan concentration in culture (panel “c”).

FIG. 5 shows that SLC7A5 (LAT1) or SLC7A11(xCT)-modified CAR-T cells maintain the capacity to kill target cancer cells on repeated exposure . Panel “a” is a cartoon demonstrating CAR-T cells and AML cells (TH P1) were incubated at a ratio of 1:5 respectively. Fresh AML cells were added at 0 hours, 48 hours, 96 hours, 128 hours. Panels b, c and d show Viability of AML cells was assessed by flow cytometry Time 1(48hours), Time 2 (96hours), time 3 (128hours), time 4 (148hours). Data for 3 individual donors shown in panels b, c and d.

FIG. 6 shows that SLC7A5 (LAT1) or SLC7A11(xCT)-modified CAR-T cells lead to increased tumour cell clearance and prolonged murine survival in vivo. Panel “a” is a cartoon demonstrating that NOG-SCID mice were engrafted with 1×10⁶ CD33+tumour cells (HL60). Once bone marrow engraftment was confirmed on femoral biopsy, 2.5×10⁶ CAR-T cells were administered by tail vein injection. Panel “b” is a graph showing LAT-1 or xCT modified CAR-T cells have enhanced clearance of tumour cells from the bone marrows compared to standard ‘no transporter’ CAR-T cells. Panel “c” is a graph showing that mice treated with LAT-1 or xCT modified CAR-T cells have prolonged survival compared to mice treated with standard ‘no transporter’ CAR-T cells. Panel “d” is a graph showing CAR-T cells are detectable within the bone marrow environment of treated mice, as measured by qRT-PCR.

FIG. 7 shows SLC7A5 (LAT1) modified CAR-T cells have a series of unique, downstream transcriptomic adaptations to the tumour microenvironment compared to unmodified CAR-T cells. Panel “a” shows a heatmap of differential expression analysis, showing the top 50 genes significantly up-/down-regulated in CD33 vs CD33-LAT1 CAR-T cells. Panel “b” shows a more detailed heatmap of transcriptomic adaptations in the glycolysis pathway in CD33 vs CD33-LAT1 CAR-T cells. Panel “c” shows a more detailed heatmap of transcriptomic adaptations in the oxidative phosphorylation pathway in CD33 vs CD33-LAT1 CAR-T cells. The genes shown in panels “b” and “c” are detailed in Table 2.

FIG. 8 shows that SLC7A11 (xCT) modified CAR-T cells have a series of unique, downstream transcriptomic adaptations to the tumour microenvironment compared to unmodified CAR-T cells. Panel “a” shows a heatmap of differential expression analysis, showing the top 50 genes significantly up-/down-regulated in CD33 vs CD33-xCT CAR-T cells. Panel “b” shows a more detailed heatmap of transcriptomic adaptations in the glycolysis pathway in CD33 vs CD33-xCT CAR-T cells. Panel “c” shows a more detailed heatmap of transcriptomic adaptations in the oxidative phosphorylation pathway in CD33 vs CD33-xCT CAR-T cells. The genes shown in panels “b” and “c” are detailed in Table 2.

FIG. 9 shows SLC7A5 (LAT1) or SLC7A11(xCT)-modified CAR-T are metabolically adapted to standard and low amino acid conditions. Representative seahorse assay for unmodified and modified CAR-T cells (A) SLC7A11(xCT) (B) SLC7A5 (LAT1). Panels C and D are pooled analysis showing unmodified CAR-T cells have lower basal, ATP linked and maximal respiratory response capacity compared to transporter modified CAR-T cells (panel C: CAR vs xCT CAR and panel D: CAR vs LAT1 CAR).

### DETAILED DESCRIPTION OF THE INVENTION

The invention is based, at least in part, on the inventors' observation that therapeutically effective cells, such as CAR-T cells, have markedly reduced expression of amino acid transporters. Furthermore, blocking the action of amino acid transporters inhibits proliferation of these cells, indicating that low endogenous expression of amino acid transporters by T cells and CAR-T cells contributes to their low proliferation noted in clinical settings.

In contrast, and as demonstrated in the Examples set out towards the end of this specification, cells of the invention (comprising a nucleic acid construct of the invention) demonstrate a dramatically improved proliferation response in the presence of cancer cells expressing a target protein recognised by the fusion target-binding protein. It will be appreciated that this increase in proliferation will offer significant clinical advantages, as the number of therapeutic cells present, and thus able to provide therapeutic activity, is much increased as compared to control or comparator CAR T cells.

Cells of the invention are shown to exhibit elevated expression of amino acid transporters. Without wishing to be bound by any hypothesis, the inventors believe that nucleic acid constructs in accordance with the invention are advantageous since the amino acid transporter that they encode allows uptake of amino acids into a cell in which it is expressed.

Cells comprising nucleic acid constructs of the invention offer a number of advantages in terms of the biological, and particularly therapeutic, activity of the cells that are well suited to increase cell activity in the immunosuppressive tumour microenvironment. Such advantages include the improved proliferation noted above, and retained cytocidal activity.

Accordingly, it will be recognised that the nucleic acid constructs and cells of the invention provide improved therapeutic agents as compared to CAR-based therapies of the prior art. The various aspects and embodiments of the invention described herein arise from, or contribute to, these improvements.

The invention will now be further described with reference to the following paragraphs.

### Nucleic Acid Constructs

Nucleic acid constructs in accordance with the present invention comprise at least first and second sequences, respectively encoding an amino acid transporter and a fusion target-binding protein. As disclosed further herein, they may optionally also comprise one or more additional sequence independently selected from the group consisting of:
Third sequences that encode a CD98 chain.
Further sequences that encode an amino acid transporter.
Cleavage sequences, optionally located between two or more of the first and second sequences, and third and further sequences (if present).

Further details of each of these optional sequences are set out below.

A nucleic acid construct of the invention may be provided in the form of a vector. Suitably the vector may be a viral vector, such as a retroviral vector or a lentiviral vector, or a transposon. Both retroviral and lentiviral approaches may be used successfully in the production of cells of the invention.

Suitably a nucleic acid construct in accordance with the invention comprises DNA. In a suitable embodiment, a nucleic acid construct of the invention comprises RNA. It will be appreciated that a suitable nucleic acid construct may essentially consist of DNA, may essentially consist of RNA, or may comprise a combination of DNA and RNA.

### First Sequences, Encoding Amino Acid Transporters

As set out above, the nucleic acid constructs of the invention comprise a first sequence encoding an amino acid transporter. Amino acid transporters that may be encoded by the first sequence are described in more detail below.

### Second Sequences, Encoding Fusion Target-Binding Proteins

The nucleic acid constructs of the invention comprise a second sequence that encodes a fusion target-binding protein, such as a CAR. Nucleic acid constructs of the invention may optionally comprise more than one copy of the second sequence. Fusion target-binding proteins that may be encoded by the second sequence are described in more detail below.

### Third Sequences, Encoding CD98

The nucleic acid constructs of the invention may optionally comprise a third sequence, that encodes a CD98 chain (also know as SLC3A2). The constructs of the invention comprise one or more copies of such a third sequence.

Suitably, a nucleic acid construct of the invention may comprise a sequence encoding the amino acid sequence of CD98 set out in SEQ ID NO: 5, or a fragment or derivative thereof. An example of a third sequence suitable for use in the nucleic acid constructs of the invention is set out in SEQ ID NO: 6, which encodes the protein of SEQ ID NO: 5. It will be appreciated that a functional fragment or variant of this sequence may be used as an alternative.

A number of amino acid transporters function as pairs of heavy and light chains. CD98 constitutes a heavy chain that may act alongside a light chain encoded by a first or further sequence of the invention.

Certain cells, and in particular leucocytes such as T cells or CAR-T cells, may lack sufficient endogenous expression of CD98 for optimal amino acid transporter activity. Accordingly, the inventors believe that the inclusion of optional third sequences, encoding CD98, may be of benefit when the nucleic acid constructs of the invention are for use in such cells.

### Further Sequences, Encoding Amino Acid Transporters

The nucleic acid constructs of the invention may optionally comprise one or more further sequences that encode an amino acid transporter. The amino acid transporter encoded by a further sequence may be the same as the amino acid transporter encoded by the first sequence, or it may be a different amino acid transporter.

A nucleic acid construct of the invention may comprise one, two, three, four, five, or more further sequences.

Each of these further sequences may encode the same amino acid transporter as the first sequence, or a different amino acid transporter. In the case that a nucleic acid construct comprises multiple further sequences, each of these may encode the same amino acid transporter, or they may encode one or more different amino acid transporters.

As noted above, the nomenclature of “first” and “second” sequences, and also “third” and “further” sequence, is used to allow distinction between the relevant sequences for the purpose of this disclosure. It should not be taken as imposing any limitation on the order of these sequences within the construct, unless the context requires otherwise. Thus a “second” sequence or a “third” or “further” sequence may be located before a “first” sequence in a nucleic acid construct of the invention.

Since each encodes a defined product, the first, second, third and further sequences may be referred to as “coding sequences” within this disclosure.

### Cleavage Sequences

Nucleic acid constructs of the invention may optionally comprise one or more cleavage sequences. These are optionally located between the sequences encoding proteins, to allow the products encoded by the first, second, third, and further sequences to be expressed as separate proteins.

Thus cleavage sequences may be provided between two or more of the first and second sequences, and third and further sequences (if present). Suitably a cleavage sequence may be provided between each adjacent coding sequence.

The P2A cleavage sequence represents a suitable example of a cleavage sequence for use in the constructs of the present invention. The DNA sequence is set out in SEQ ID NO: 9.

### Fragments or Variants of Sequences in the Context of the Present Invention

The specification contains a number of exemplary nucleic acid and protein sequences. These include sequences of amino acid transporters, fusion target-binding proteins, the CD98 chain, and cleavage sequences, as well as of nucleic acids encoding these products.

It will be appreciated that, except for where the context requires otherwise, the scope of the invention should not be limited to the specific exemplary sequences set out herein. In particular, the skilled reader will recognise that fragments or variants of the exemplary sequences may still be able to provide the required activity conferred by the exemplary sequences. Such suitable fragments or variants of the exemplary sequences may be utilised in the various aspects and embodiments of the invention.

In the context of the present invention, a fragment of a sequence should be taken as being a truncated version of the original sequence (i.e. not the full length sequence), but as sharing full sequence identity with a corresponding portion of the original sequence.

In contrast a variant of a sequence should be taken as being a protein or nucleic acid that shares a certain degree of identity with the original sequence (or with a particular fragment of the original sequence) but that includes at least one modification (for example, a substitution, addition, or deletion) as compared to the original sequence.

Accordingly, references in the present specification to exemplary amino acid or nucleic acid sequences should, except for where the context requires otherwise, be taken as also encompassing functional fragments or variants of the exemplary sequences. For example, a suitable fragment may comprise at least 60%, at least 65%, at least 70%, at least 75%, at least 80%, at least 85%, at least 90%, or at least 95% of the full length of a relevant exemplary sequence. Indeed, a suitable variant may comprise at least 96%, at least 97%, at least 98%, or at least 99% of the full length of the exemplary sequence.

A suitable variant may share at least 60%, at least 65%, at least 70%, at least 75%, at least 80%, at least 85%, at least 90%, or at least 95% identity with a relevant exemplary sequence. Indeed, a suitable variant may share at least 96%, at least 97%, at least 98%, or at least 99% identity with the relevant exemplary sequence.

That a fragment or variant is “functional” may be assessed experimentally, with reference to assays known to those skilled in the art, including those assays described in the Examples. These include assays for the assessment of amino acid transport and for binding and subsequent biological activities (e.g. cytocidal activity, activation, and proliferation) of fusion target-binding proteins, such as CARs. In the case of components of the fusion target-binding proteins of the invention, such as target binding moieties, function may be determined with reference to the ability to bind a desired target.

### Amino Acid Transporters That May Be Encoded By First or Further Sequences of the Invention

The first sequence of a nucleic acid construct of the invention, and any further sequences (if present) encode amino acid transporters. The amino acid transporter encoded by the first sequence, and any further transporter encoded by the further sequence if present, may be selected from the group consisting of: the SLC7A5 gene product, or a fragment or variant thereof; the SLC7A11 gene product, or a fragment or variant thereof; and the SLC7A1 gene product, or a fragment or variant thereof; or the gene product, or a fragment or derivative thereof, of any of the genes set out in Table 1. In the event that multiple further sequences are present the amino acid transporters that these encode may be independently selected from the group set out above.

The amino acid transporter encoded by a nucleic acid construct of the invention functions to import amino acids into a cell comprising the construct. This is advantageous, since many therapeutically important cells, such as leucocytes (including T cells and CAR-T cells) have been found to suffer from significantly reduced amino acid transporter expression. This lack of functional amino acid transporters contributes to poor performance (including biological activities such as cytocidal activity, proliferation, and persistence) that is exhibited by many such cells, and is exacerbated in conditions (such as those found in the tumour microenvironment) where amino acid concentration is low.

An amino acid transporter encoded by a nucleic acid construct of the invention may one that functions as a “light chain” in partnership with a “heavy chain”, such as CD98.

Of particular interest as amino acid transporters to be encoded by the nucleic acid constructs of the invention are the SLC7A5 gene product, or fragments or variants thereof; the SLC7A11 gene product, or fragments or variants thereof; and the SLC7A1 gene product, or fragments or variant thereof.

Suitably, a nucleic acid construct of the invention may comprise a sequence encoding the SLC7A5 gene product (the amino acid sequence of which is set out in SEQ ID NO: 3), or a fragment or derivative thereof. The nucleic acid sequence may comprise the DNA sequence set out in SEQ ID NO: 4, or a fragment or variant of this sequence.

Suitably, a nucleic acid construct of the invention may comprise a sequence encoding the SLC7A11 gene product (the amino acid sequence of which is set out in SEQ ID NO: 7), or a fragment or derivative thereof. The nucleic acid sequence may comprise the DNA sequence set out in SEQ ID NO: 8, or a fragment or variant of this sequence.

Suitably, a nucleic acid construct of the invention may comprise a sequence encoding the SLC7A1 gene product (the amino acid sequence of which is set out in SEQ ID NO: 1), or a fragment or derivative thereof. The nucleic acid sequence may comprise the DNA sequence set out in SEQ ID NO: 2, or a fragment or variant of this sequence.

SLC7A5 is known to function as a transporter of large neutral amino acids (such as those selected from the group consisting of tryptophan, phenylalanine, tyrosine, leucine and arginine). In view of this, and the effectiveness of cells and nucleic acids of the invention incorporating SLC7A5 (as discussed further elsewhere in the specification), the skilled person will appreciate that a suitable amino acid transporter to be encoded in a nucleic acid construct of the invention may be an amino acid transporter capable of transporting large neutral amino acids (such as those selected from the group consisting of tryptophan, phenylalanine, tyrosine, leucine and arginine), and in particular tryptophan. Alternative amino acid transporters having this activity, and hence suitable for use in the context of nucleic acid constructs or cells of the invention are set out in Table 1.

SLC7A11 is known to function as a transporter of anionic amino acids including cysteine and glutamate. In view of this, and the effectiveness of cells and nucleic acids of the invention incorporating SLC7A11 (as discussed further elsewhere in the specification), the skilled person will appreciate that a suitable amino acid transporter to be encoded in a nucleic acid construct of the invention may be an amino acid transporter capable of transporting anionic amino acids. A suitable amino acid transporter to be encoded in a nucleic acid construct of the invention may be an amino acid transporter capable of transporting cysteine and/or capable of transporting glutamate. Alternative amino acid transporters having this activity, and hence suitable for use in the context of nucleic acid constructs or cells of the invention, are set out in Table 1.

SLC7A1 is known to function as a transporter of cationic amino acids (such as those selected from the group consisting of arginine, lysine and ornithine). In view of this, the skilled person will appreciate that a suitable amino acid transporter to be encoded in a nucleic acid construct of the invention may be an amino acid transporter capable of transporting cationic amino acids (such as those selected from the group consisting of arginine, lysine and ornithine). Alternative amino acid transporters having this activity, and hence suitable for use in the context of nucleic acid constructs or cells of the invention are set out in Table 1.

Without limitation, suitable examples of alternative amino acid transporters may be selected from the group consisting of: SLC1A5 and SLC6A14.

Fusion target-binding proteins that may be encoded by second sequences of the invention

Fusion target-binding proteins are artificial fusion proteins that enable a desired specificity to be conferred on desired biological properties of a cell by which the protein of the invention is expressed. Perhaps the best known examples of such proteins are CARs—chimeric antigen receptors.

Whether or not a nucleic acid sequence encodes a fusion target-binding protein (whether generally, or with reference to a particular fusion target-binding protein of interest) may readily be resolved by determining the protein encoded by a nucleic acid sequence of interest, and comparing this protein with known databases of fusion target-binding proteins (such as CARs).

Different types of cells, and the desired biological properties that they are respectively able to provide in the context of the present invention, are discussed further elsewhere in the specification. Typically, in the context of medical uses of cells expressing fusion target-binding proteins, cytocidal activity targeted against cells associated with a disease (such as cancer cells or infected cells) confers the required therapeutic utility.

Fusion target-binding proteins suitable for use in the invention may comprise a target binding moiety and an intracellular signalling region. These terms are defined elsewhere within the present specification. The skilled person will appreciate that such proteins may also incorporate various other optional domains or regions.

The different portions of the fusion target-binding protein (e.g. target binding moieties and intracellular signalling regions) may be derived from two or more different “sources”. Thus, the different portions may be derived from two or more naturally occurring molecules, such as proteins. Additionally, the different portions may be derived from different sources in terms of different originating kingdoms or species.

A class of fusion target-binding proteins of particular interest in the context of the present invention are chimeric antigen receptor (CAR) proteins. CARs utilise antibodies, or fragments thereof, to confer specificity of binding, and intracellular signalling regions to determine the specific biological activity required. Various different generations of CARs are known, and each of these different generations represents a suitable example of a fusion target-binding protein for use in the invention, unless the context of the present disclosure requires otherwise.

For the avoidance of doubt, fusion target-binding proteins suitable for use in the context of the invention may also be taken as encompassing T cell receptors (TCRs) that have been modified to confer a desired specificity for a target protein of interest. In such embodiments, the target binding moiety may be provided by the TCR a and TCR R chains of the receptor. Since they comprise both “natural” and “artificial” sequence elements, such modified TCRs are considered fusion proteins for the purposes of the present invention.

Fusion target-binding proteins for use in the invention typically further comprise additional portions, including one or more from the group consisting of: a transmembrane portion, a CH2CH3 spacer portion, a CD8 hinge portion, and a CD8a signalling portion.

The amino acid sequences of exemplary fusion target-binding proteins that may be encoded by second sequences in the nucleic acid constructs of the invention are set out in SEQ ID NOs: 10 to 13. It will be appreciated that a molecule comprising or consisting of any of these sequences represents a suitable fusion target-binding protein in the context of the present invention.

Suitably the fusion target-binding protein encoded by the second sequence in a nucleic acid construct of the invention is selected from the group consisting of: an anti-GD2 fusion target- binding protein; an anti-CD33 fusion target-binding protein; an anti-mesothelin fusion target-binding protein; an EGFRvIII fusion target-binding protein; an anti-VEGFR2 fusion target-binding protein; an anti-FAP fusion target-binding protein; an anti-EpCam fusion target-binding protein; an anti-GPC3 fusion target-binding protein; an anti-CD133 fusion target-binding protein; an anti-IL13Ra fusion target-binding protein; an anti-EphA2 fusion target-binding protein; an anti-Muc1 fusion target-binding protein; an anti-BCMA fusion target-binding protein; an anti-CD70 fusion target-binding protein; an anti-CD123 fusion target-binding protein; an anti-ROR1 fusion target-binding protein; an anti-PSMA fusion target-binding protein; an anti-CD5 fusion target-binding protein; an anti-GAP fusion target-binding protein; an anti-CEA fusion target-binding protein; an anti-PSCA fusion target-binding protein; an anti-Her2 fusion target-binding protein; and an anti-CD19 fusion target-binding protein.

Target binding moieties of fusion target-binding proteins

The fusion target-binding proteins encoded by the constructs of the invention typically comprise a target binding moiety. Suitably the target binding moiety is an extracellular target binding moiety. Such moieties are particularly suitable for binding a target that is extracellular (with reference to the cell expressing the fusion target-binding protein).

The target binding moiety confers specificity of binding of the proteins (and hence of the cytocidal activity of the cells comprising nucleic acid constructs of the invention), to target structures, such as cells, on which a target molecule, recognised by the target binding moiety, is found.

In particular, the target binding moieties confer specificity of the biological activities of the cells of the invention (for example, cytocidal activity, or cell proliferation in response to activation) that underpin their therapeutic utility. Except for where the context requires otherwise, references to specific binding in the present disclosure may be interpreted as referring to a target binding moiety's ability to discriminate between possible partners in the environment in which binding is to occur. A target binding moiety that interacts with one particular target molecule when other potential targets are present is said to “bind specifically” to the target molecule with which it interacts. In some embodiments, specific binding is assessed by detecting or determining degree of association between the target binding moiety and its target molecule; in some embodiments, specific binding is assessed by detecting or determining degree of dissociation of a binding moiety-target molecule complex; in some embodiments, specific binding is assessed by detecting or determining ability of the target binding moiety to compete an alternative interaction between its target molecule and another entity. In some embodiments, specific binding is assessed by performing such detections or determinations across a range of concentrations, In a suitable embodiment, specific binding is assessed by determining the difference in binding affinity between cognate and non-cognate targets. For example, a target binding moiety that is specific may have a binding affinity for a cognate target molecule that is about 3 -fold, 4-fold, 5-fold, 6-fold, 7-fold, 8-fold, 9- fold, 10-fold or more than binding affinity for a non-cognate target,

In the context of the present disclosure, “specificity” is a measure of the ability of a particular target binding moiety to distinguish its target molecule binding partner from other potential binding partners.

A suitable target binding moiety may be directed to any desired target molecule. The target binding moiety may be directed to a target molecule expressed exclusively, or extensively, by a target against which it is desired to direct the cytocidal activity of a cell expressing a protein of the invention. For example, a target binding moiety may be directed to a target molecule associated with a disease. Suitably the target binding moiety may be directed to a target molecule associated with cancer, or with an infection.

In a suitable embodiment, the target binding moiety is selected from the group consisting of: a GD2 target binding moiety; a CD33 target binding moiety; a mesothelin target binding moiety; an EGFRvIII target binding moiety; a VEGFR2 target binding moiety; a FAP target binding moiety; a EpCam target binding moiety; a GPC3 target binding moiety; a CD133 target binding moiety; a IL13Ra target binding moiety; a EphA2 target binding moiety; a Muc1 target binding moiety; a BCMA target binding moiety; a CD70 target binding moiety; a CD123 target binding moiety; a ROR1 target binding moiety; a PSMA target binding moiety; a CD5 target binding moiety; a GAP target binding moiety; a CEA target binding moiety; a PSCA target binding moiety; a Her2 target binding moiety; and a CD19 target binding moiety.

Examples of suitable GD2, CD33, mesothelin, and EGFRvIII target binding moieties are set out in SEQ ID NOs: 14 to 18. It will be appreciated that fragments or variants (for example, variants differing from the exemplary sequence by 1, 2, 3, 4, 5, or more amino acid residues) may be used as alternative target binding moieties, as long as the fragment or variant retains the ability to bind the target molecule.

Without limitation, suitable target binding moieties may be selected from the group consisting of: antibodies; antibody fragments (such as scFvs); derivatives of antibodies or their fragments; TCRs, such as TCR a chains or TCR R chains; and aptamers.

### A GD2 Target Binding Moiety

A GD2 target binding moiety is a moiety capable of binding to disialoganglioside 2 (GD2), which may also be referred to as ganglioside GD2. A protein comprising a GD2 target binding moiety is suitable for use in circumstances in which it is desired to exert the cytocidal activity of a cell expressing a protein of the invention against a target comprising GD2 molecules, for example a cell expressing GD2.

GD2 is expressed by cancers of neuroectodermal origin, including neuroblastoma, osteosarcoma and melanoma. Therefore, it will be appreciated that a protein (such as a CAR) comprising a GD2 target binding moiety is suitable for use in circumstances in which it is desired to utilise a cell of the invention in a medical use for the prevention and/or treatment of any such GD2 expressing cancers, and particularly neuroblastoma.

A GD2 target binding moiety suitable for incorporation in such a protein may be an anti-GD2 antibody, such as an anti-GD2 monoclonal antibody, or an antigen binding fragment or derivative thereof. For example, a GD2 target binding moiety may be an anti-GD2 scFv antibody fragment. Merely by way of example, a suitable GD2 targeting domain comprising an scFv antibody fragment is set out in SEQ ID NO: 14.

The scFv antibody fragment set out in SEQ ID NO: 14 is also referred to as the 14g2a scFv, as described in U.S. Pat. No. 9,493,740 B2. It is derived from the ch14.18 antibody disclosed in U.S. Pat. No. 9,777,068 B2, and it will be appreciated that other ch14.18 antibody fragments or variants may be used as GD2 target binding moieties.

Alternatively, a suitable GD2 target binding moiety may be selected from the group consisting of: an anti-GD2 aptamer; or a fragment or derivative thereof.

Suitably a GD2 target binding moiety is capable of binding specifically to GD2.

### A CD33 Target Binding Moiety

A CD33 target binding moiety is a moiety capable of binding to CD33 (also known as Siglec-3). CD33 is transmembrane protein. A protein comprising a CD33 target binding moiety is suitable for use in circumstances in which it is desired to exert the biological activity of a cell of the invention against a target comprising CD33 molecules, for example a cell expressing CD33.

CD33 is expressed by acute myeloid leukaemia (AML) cells. Therefore, it will be appreciated that a protein comprising a CD33 target binding moiety is suitable for use in circumstances in which it is desired to utilise a cell of the invention in a medical use for the prevention and/or treatment of a CD33 expressing cancer, such as AML.

A CD33 target binding moiety suitable for incorporation in such a protein may be an anti-CD33 antibody, such as an anti-CD33 monoclonal antibody, or an antigen binding fragment or derivative thereof. For example, a CD33 target binding moiety may be an anti-CD33 scFv antibody fragment. Merely by way of example, a suitable CD33 targeting domain comprising an scFv antibody fragment is set out in SEQ ID NO: 15.

The scFv antibody fragment is set out in SEQ ID NO: 15 is derived from the humanised my96 clone monoclonal antibody. Details of the my96 antibody are set out in Leukemia. 2015 August;29(8):1637-47, and details of the scFv fragment of SEQ ID NO: 15 are set out in US20160096892A1 (where this scFv is disclosed as SEQ ID NO: 147). It will be appreciated that other my96 antibody fragments or variants may be used as CD33 target binding moieties.

Alternatively, a suitable CD33 target binding moiety may be selected from the group consisting of: an anti-CD33 aptamer; or a fragment or derivative thereof.

Suitably a CD33 target binding moiety is capable of binding specifically to CD33.

Examples of nucleic acid constructs of the invention comprising a CD33 target binding moiety are set out in SEQ ID NO: 20 and SEQ ID NO: 22.

### A Mesothelin Target Binding Moiety

A mesothelin target binding moiety is a moiety capable of binding to mesothelin. Mesothelin is a 40 kDa protein that is the product of the MSLN. A protein comprising a mesothelin target binding moiety is suitable for use in circumstances in which it is desired to exert the biological activity of a cell of the invention against a target comprising mesothelin molecules, for example a cell expressing mesothelin.

Mesothelin is expressed by cells of a number of different types of cancers. Mesothelin expressing cancers include, for example, epithelial cancers, such as ovarian cancer, lung adenocarcinoma, and pancreatic cancer. Therefore, it will be appreciated that a protein comprising a mesothelin target binding moiety is suitable for use in circumstances in which it is desired to utilise a protein of the invention in a medical use for the prevention and/or treatment of any mesothelin expressing cancer.

A mesothelin target binding moiety suitable for incorporation in such a protein may be an anti-mesothelin antibody, such as an anti-mesothelin monoclonal antibody, or an antigen binding fragment or derivative thereof. For example, a mesothelin target binding moiety may be an anti-mesothelin scFv antibody fragment. Merely by way of example, a suitable mesothelin targeting domain comprising an scFv antibody fragment is set out in SEQ ID NO: 16.

The scFv antibody fragment is set out in SEQ ID NO: 16 is derived from the SS1 antibody. Details of this antibody, and an scFV derived therefrom, are set out in WO 2015/090230 A (where the amino acid sequence of murine SS1 scFv is provided in SEQ ID NO: 279). It will be appreciated that other SS1 antibody fragments or variants may be used.

Alternatively, a suitable mesothelin target binding moiety may be selected from the group consisting of: an anti-mesothelin aptamer; or a fragment or derivative thereof.

Suitably a GD2 target binding moiety is capable of binding specifically to GD2.

### An EGFRvIII Target Binding Moiety

A EGFRvIII target binding moiety is a moiety capable of binding to epidermal growth factor receptor variant III (EGFRvIII). A protein comprising a EGFRvIII target binding moiety is suitable for use in circumstances in which it is desired to exert the biological activity of a cell of the invention against a target comprising EGFRvIII molecules, for example a cell expressing EGFRvIII.

EGFRvIII is expressed by a range of cancers of epithelial origin. Therefore, it will be appreciated that a protein comprising an EGFRvIII target binding moiety is suitable for use in circumstances in which it is desired to utilise a cell of the invention in a medical use for the prevention and/or treatment of cancers expressing EGFR, such as glioblastomas, and colorectal cancers. In particular, a protein comprising an EGFRvIII target binding moiety is suitable for use in the prevention and/or treatment of an EGFRvIII expressing cancer, such as glioblastoma.

An EGFRvIII target binding moiety suitable for incorporation in such a protein may be an anti-EGFRvIII antibody, such as an anti-EGFRvIII monoclonal antibody, or an antigen binding fragment or derivative thereof. For example, a EGFRvIII target binding moiety may be an anti-EGFRvIII scFv antibody fragment. Merely by way of example, a suitable EGFRvIII targeting domain comprising an scFv antibody fragment is set out in SEQ ID NO: 17.

The scFv antibody fragment is set out in SEQ ID NO: 17 is derived from the 139 antibody disclosed in WO 2012/138475 A1 (in which a human scFV of the 139 antibody is set out as SEQ ID NO: 5, and a CAR construct incorporating the scFv is set out as SEQ ID NO: 11). It will be appreciated that other 139 antibody fragments or variants may be used as mesothelin target binding moieties.

An alternative EGFRvIII target binding moiety may be derived from the MR1 anti-EGFRvIII antibody. An example of such an EGFRvIII target binding moiety is the scFv (derived from MR1) of SEQ ID NO: 18, encoded by the DNA sequence of SEQ ID NO: 19.

Alternatively, a suitable EGFRvIII target binding moiety may be selected from the group consisting of: an anti-EGFRvIII aptamer; or a fragment or derivative thereof.

Suitably a EGFRvIII target binding moiety is capable of binding specifically to EGFRvIII.

### Intracellular Signalling Regions

Fusion target-binding proteins encoded by constructs of the invention may comprise at least one intracellular signalling region. The intracellular signalling region serves to couple binding of the target binding moiety to a target molecule with other biological activities of the cell expressing the protein. In particular, a suitable intracellular signalling region may couple binding of the target binding moiety to its target molecule with activation of the cell's cytocidal activity and/or to the cells ability to proliferate in response to activation.

A suitable intracellular signalling region may activate cytotoxic or specific cytolytic activity in response to binding of the target molecule to the target binding moiety. Alternatively, or additionally, a suitable intracellular signalling region may facilitate activation-induced cell proliferation in response to binding of the target molecule to the target binding moiety.

In a suitable embodiment, an intracellular signalling region comprises a region selected from the group consisting of: a 4-1BB signalling region; an OX-40 signalling region; a CD28 signalling region; an ICOS signalling region; and a CD3 signalling region.

It will be appreciated that suitable proteins may comprise a plurality of intracellular signalling regions. Suitably the plurality may comprise more than one copy of an individual intracellular signalling region. For example, a protein may comprise multiple copies of one, or more, of: a 4-1BB signalling region; an OX-40 signalling region; a CD28 signalling region; an ICOS signalling region; and a CD3 signalling region.

Additionally, or alternatively, a protein comprise a combination of multiple intracellular signalling regions. For example, a protein may comprise a combination of intracellular signalling regions selected from the group consisting of: a 4-1BB signalling region; an OX-40 signalling region; a CD28 signalling region; an ICOS signalling region; and a CD3 signalling region. Merely by way of example, a protein may comprise both a 4-1BB signalling region and a CD3 signalling region.

### Exemplary Nucleic Acid Constructs of the Invention

SEQ ID NO: 20 and SEQ ID NO: 22 represent exemplary nucleic acid constructs of the invention.

Accordingly, in a suitable embodiment, a nucleic acid construct of the invention comprises the nucleic acid sequence of SEQ ID NO: 20. In a suitable embodiment, a nucleic acid construct of the invention consists of the nucleic acid sequence of SEQ ID NO: 20. In a suitable embodiment a nucleic acid construct of the invention comprises a variant of a SEQ ID NO: 20.

in a suitable embodiment, a nucleic acid construct of the invention comprises the nucleic acid sequence of SEQ ID NO: 22. In a suitable embodiment, a nucleic acid construct of the invention consists of the nucleic acid sequence of SEQ ID NO: 22. In a suitable embodiment a nucleic acid construct of the invention comprises a variant of a SEQ ID NO: 20.

The nucleic acid constructs of SEQ ID NO: 20 and SEQ ID NO: 22 encode an anti-CD33 CAR as a fusion target-binding protein, and (respectively) SLC7A5 and SLC7A11 as an amino acid transporter. Each of these nucleic acid constructs also incorporates a number of different coding sequences.

In the case of SEQ ID NO: 20, the nucleic acid construct encodes:
1. tCD34
2. F2A
3. CD8 signal peptide
4. CD8 hinge
5. 4-1BB
6. CD3z signalling domain
7. P2A
8. SLC7A5 (also referred to as LAT1).

In the case of SEQ ID NO: 22, the nucleic acid construct encodes:

1. tCD34
2. F2A
3. CD8 signal peptide
4. CD8 hinge
5. 4-1BB
6. CD3z signalling domain
7. P2A
8. SLC7A11 (also referred to as xCT).

In each case 2 and 7 (F2A and P2A) comprise cleavage sites, and 3-6 are the constituents of the anti-CD33 CAR.

For the purposes of the present disclosure, a variant of the nucleic acid construct of SEQ ID NO: 20 or a variant of a nucleic acid construct of SEQ ID NO: 22 may be considered to be a nucleic acid construct that encodes the same constituents as SEQ ID NO: 20 or SEQ ID NO:

22.

Suitably a variant of SEQ ID NO: 20 may comprise a coding sequence at least 80% identical to the tCD34 encoding sequence set out in SEQ ID NO: 20. Suitably a variant of SEQ ID NO: may comprise a coding sequence at least 85%, at least 90%, or at least 95%, at least 96%, at least 97%, at least 98% or at least 99% identical to the tCD34 encoding sequence set out in SEQ ID NO: 20.

Suitably a variant of SEQ ID NO: 20 may comprise a coding sequence at least 80% identical to the F2A encoding sequence set out in SEQ ID NO: 20. Suitably a variant of SEQ ID NO: may comprise a coding sequence at least 85%, at least 90%, or at least 95%, at least 96%, at least 97%, at least 98% or at least 99% identical to the F2A encoding sequence set out in SEQ ID NO: 20.

Suitably a variant of SEQ ID NO: 20 may comprise a coding sequence at least 80% identical to the CD8 signal peptide encoding sequence set out in SEQ ID NO: 20. Suitably a variant of SEQ ID NO: 20 may comprise a coding sequence at least 85%, at least 90%, or at least 95%, at least 96%, at least 97%, at least 98% or at least 99% identical to the CD8 signal peptide encoding sequence set out in SEQ ID NO: 20.

Suitably a variant of SEQ ID NO: 20 may comprise a coding sequence at least 80% identical to the CD33 ScFV encoding sequence set out in SEQ ID NO: 20. Suitably a variant of SEQ ID NO: 20 may comprise a coding sequence at least 85%, at least 90%, or at least 95%, at least 96%, at least 97%, at least 98% or at least 99% identical to the CD33 ScFV encoding sequence set out in SEQ ID NO: 20.

Suitably a variant of SEQ ID NO: 20 may comprise a coding sequence at least 80% identical to the CD8 hinge encoding sequence set out in SEQ ID NO: 20. Suitably a variant of SEQ ID NO: 20 may comprise a coding sequence at least 85%, at least 90%, or at least 95%, at least 96%, at least 97%, at least 98% or at least 99% identical to the CD8 hinge encoding sequence set out in SEQ ID NO: 20.

Suitably a variant of SEQ ID NO: 20 may comprise a coding sequence at least 80% identical to the 4-1BB encoding sequence set out in SEQ ID NO: 20. Suitably a variant of SEQ ID NO: may comprise a coding sequence at least 85%, at least 90%, or at least 95%, at least 96%, at least 97%, at least 98% or at least 99% identical to the 4-1BB encoding sequence set out in SEQ ID NO: 20.

Suitably a variant of SEQ ID NO: 20 may comprise a coding sequence at least 80% identical to the CD3z signalling domain encoding sequence set out in SEQ ID NO: 20. Suitably a variant of SEQ ID NO: 20 may comprise a coding sequence at least 85%, at least 90%, or at least 95%, at least 96%, at least 97%, at least 98% or at least 99% identical to the CD3z signalling domain encoding sequence set out in SEQ ID NO: 20.

Suitably a variant of SEQ ID NO: 20 may comprise a coding sequence at least 80% identical to the P2A encoding sequence set out in SEQ ID NO: 20. Suitably a variant of SEQ ID NO: may comprise a coding sequence at least 85%, at least 90%, or at least 95%, at least 96%, at least 97%, at least 98% or at least 99% identical to the P2A encoding sequence set out in SEQ ID NO: 20.

Suitably a variant of SEQ ID NO: 20 may comprise a coding sequence at least 80% identical to the SLC7A5 encoding sequence set out in SEQ ID NO: 20. Suitably a variant of SEQ ID NO: 20 may comprise a coding sequence at least 85%, at least 90%, or at least 95%, at least 96%, at least 97%, at least 98% or at least 99% identical to the SLC7A5 encoding sequence set out in SEQ ID NO: 20.

Suitably a variant of SEQ ID NO: 22 may comprise a coding sequence at least 80% identical to the tCD34 encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the tCD34 encoding sequence set out in SEQ ID NO: 22.

Suitably a variant of SEQ ID NO: 22 may comprise a coding sequence at least 80% identical to the F2A encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the F2A encoding sequence set out in SEQ ID NO: 22.

Suitably a variant of SEQ ID NO: 22 may comprise a coding sequence at least 80% identical to the CD8 signal peptide encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the CD8 signal peptide encoding sequence set out in SEQ ID NO: 22.

Suitably a variant of SEQ ID NO: 22 may comprise a coding sequence at least 80% identical to the CD33 ScFV encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the CD33 ScFV encoding sequence set out in SEQ ID NO: 22.

Suitably a variant of SEQ ID NO: 22 may comprise a coding sequence at least 80% identical to the CD8 hinge encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the CD8 hinge encoding sequence set out in SEQ ID NO: 22.

Suitably a variant of SEQ ID NO: 22 may comprise a coding sequence at least 80% identical to the 4-1BB encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the 4-1BB encoding sequence set out in SEQ ID NO: 22.

Suitably a variant of SEQ ID NO: 22 may comprise a coding sequence at least 80% identical to the CD3z signalling domain encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the CD3z signalling domain encoding sequence set out in SEQ ID NO: 22.

Suitably a variant of SEQ ID NO: 22 may comprise a coding sequence at least 80% identical to the P2A encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the P2A encoding sequence set out in SEQ ID NO: 22.

Suitably a variant of SEQ ID NO: 22 may comprise a coding sequence at least 80% identical to the SLC7A11 encoding sequence set out in SEQ ID NO: 22. Suitably a variant of SEQ ID NO: 22 may comprise a coding sequence at least 85%, at least 90%, or at least 95%, at least 96%, at least 97%, at least 98% or at least 99% identical to the SLC7A11 encoding sequence set out in SEQ ID NO: 22.

### Exemplary Polypeptides of the Invention

As set out above, the invention provides polypeptides that are the expression product of a nucleic acid construct in accordance with the first aspect of the invention. Amino acid sequences of examples of such polypeptides are set out in SEQ ID NO: 21 and SEQ ID NO:22.

A polypeptide sequence of the invention may comprise the amino acid sequence of SEQ ID NO: 21, or the amino acid sequence of SEQ ID NO: 23. A polypeptide of the invention may consist of the amino acid sequence of SEQ ID NO: 21, or the amino acid sequence of SEQ ID NO: 23.

A polypeptide sequence of the invention may share at least 75%, at least 80%, at least 85%, at least 90%, at least 95%, or at least 96%, 97%, 98% or 99% identity with the amino acid sequence of SEQ ID NO: 21.

A polypeptide sequence of the invention may share at least 75%, at least 80%, at least 85%, at least 90%, at least 95%, or at least 96%, 97%, 98% or 99% identity with the amino acid sequence of SEQ ID NO: 23.

Cells of the invention comprising such a polypeptide sequence may also be characterised with references to the definitions set out above.

### Cells of the Invention

The second aspect of the invention provides a cell comprising a nucleic acid construct in accordance with the first aspect of the invention.

Suitably a cell in accordance with the second aspect of the invention is a cell able to exert a cell-mediated immune response. A suitable cell may be able to exert cytocidal activity, for example by cytotoxic action, or by inducing specific cell lysis. Additionally, a suitable cell may be able to proliferate in response to binding of the protein to its corresponding target molecule. Suitably, a cell in accordance with the second aspect of the invention may be selected from the group consisting of: a T cell; and a natural killer (NK) cell.

Suitably a T cell may be selected from the group consisting of: an invariant natural killer T cell (iNKT); a natural killer T cell (NKT); a gamma delta T cell (gd T cell); an alpha beta T cell (ab T cell); an effector T cell; and a memory T cell.

Suitably a T cell may be selected from the group consisting of: a CD4⁺ lymphocyte; and a CD8⁺ lymphocyte.

The cell may be from a subject requiring prevention and/or treatment of a disease, such as cancer. The cell may be taken from a sample from such a subject.

Alternatively, the cell may be from a healthy donor subject (for the purposes of the present disclosure taken as a subject not afflicted with the disease to be treated with the protein or cell of the invention).

It will be appreciated that suitable cells may also include cells of cell lines.

Standard techniques for the collection of human cells, and their transformation with nucleic acids such as the constructs of the invention, are well known to those skilled in the art. Preferred techniques for the retroviral transduction of human T cells, determination of transduction efficiency, and sorting of transduced T cells by magnetic activated cell sorting, are described further in the Examples.

### Biological Activity of Cells of the Invention

Cells of the invention, comprising constructs of the invention exhibit a number of activities that are of benefit in applications such as the prevention and/or treatment of diseases.

These biological activities may be further considered with reference to cytocidal activities (which represent the means by which the cells of the invention are able to exert their therapeutic effects) and activities such as proliferation (for example in response to activation) which may contribute to increased persistence in vivo, thereby enabling the cells of the invention to exert their therapeutic effects for longer than has been the case for CAR-expressing cells of the prior art. As demonstrated in the Examples, cells of the invention demonstrate increased proliferation in response to cancer cells, and in amino acid depleted conditions, than do control or comparator CAR T cells. The Examples also illustrate that the cells of the invention demonstrate increased activation in response to cancer cells (in amino acid depleted conditions) than do control or comparator CAR T cells.

### Manufacture of Cells of the Invention

The skilled person will be aware of suitable methods by which nucleic acids, such as the nucleic acids constructs of the invention, may be used to manufacture transduced cells expressing proteins. Such methods may be used in the manufacture of cells of the invention, which comprise nucleic acid constructs of the invention. Without limitation, these include methods by which nucleic acids of the invention are introduced to cells by means such as viruses or nanoparticles.

The invention provides a method of manufacturing a therapeutically modified cell, the method comprising providing a cell with a nucleic acid sequence encoding a fusion target-binding protein and a nucleic acid sequence encoding an amino acid transporter such that the nucleic acid sequences are expressed by the cell to produce an amino acid transporter and a fusion target-binding protein. It will be appreciated that this aspect of the invention is not limited to methods utilising the nucleic acid constructs of the invention, though the use of these constructs does constitute a preferred embodiment of the methods of the invention.

In such an embodiment, a method of the invention comprises providing a cell with a nucleic acid construct according to the first aspect of the invention, such that the nucleic acid construct is expressed by the cell to produce an amino acid transporter and a fusion target-binding protein. The nucleic acid construct of the first aspect of the invention may be in accordance with any embodiment described herein. For example, the construct may comprise a further amino acid transporter and/or a CD98 chain. In this case the method is performed such that these additional elements are also expressed by the cell.

Suitably the cell may be a leukocyte, such as a T cell. Methods that are conventional in the manufacture of CAR-T cells may be utilised in the methods of the invention. Suitable methods may involve some or all of the following steps: T cell selection; T cell activation; provision of the nucleic acid to activated T cells; expansion of T cell numbers; and formulation into a pharmaceutical composition in accordance with the fourth aspect of the invention. Such a composition may then be preserved, for example by cryopreservation, until provided to a subject requiring treatment.

It will be appreciated that a cell expressing a non-naturally occurring therapeutic component (for example a fusion target-binding protein, such as a CAR, capable of binding to and killing cells expressing a target molecule) will represent a therapeutically modified cell for the purposes of the present invention.

### Cytocidal Activity of Cells of the Invention

For the purposes of the present invention, cytocidal activity should be taken as encompassing any activity by which cells of the invention kill other cells. By way of example, the killing of other cells may be achieved by means of cytotoxic action of the cells of the invention, or by specific cell lysis mediated by the cells of the invention.

Suitably, the cells of the invention exert their cytocidal activity in respect of a target that comprises target molecules bound by the fusion target binding proteins expressed by cells comprising the nucleic acid construct of the invention. Suitably, the target is a cell. Suitably a target molecule is a target protein. Suitably the cell expresses the target protein.

Suitably the cells of the invention demonstrate cytocidal activity towards cells expressing a target protein recognised by the fusion target-binding protein.

Preferably the cells killed by cytocidal activity of cells of the invention are cells associated with a disease. Suitably the cells associated with a disease may be cancer cells.

Suitably the cells of the invention demonstrate cytocidal activity towards cancer cells expressing a target protein recognised by the fusion target-binding protein.

As set out in the Examples, the inventors have demonstrated that cells of the invention (comprising the nucleic acid construct of the invention) exhibit cytocidal activity that is specifically directed to cells expressing target molecules bound by the fusion target binding proteins expressed by cells comprising the nucleic acid construct of the invention. The extent of cytocidal activity observed in respect of the cells of the invention is broadly in line with that of protein expressing cells described in the prior art, such as comparator CAR T-cells. Therefore the cells of the invention are not adversely impacted by comprising the nucleic acid construct, they maintain good cytocidal activity. However, the combination of this maintained cytocidal activity, with improved proliferation and/or persistence, exhibited by the cells of the invention confers therapeutic benefits not noted in respect of the cells of the prior art.

Suitably the cells of the invention demonstrate cytocidal activity towards cells expressing a target protein recognised by the fusion target-binding protein, wherein the activity is comparable to that of comparator CAR T-cells.

The skilled person will be aware of many suitable assays by which the cytocidal activity, whether cytotoxic activity or specific cell lysis, of a cell of the invention, or suitable comparator CAR T cell (such as an unmodified CAR T-Cell) may be assessed. Merely by way of example suitable assays are described in the Examples (point 2.2 of the examples), where they are used in the characterisation of exemplary cells of the invention.

The skilled reader, on considering the information set out in the Examples, will recognise that the cells of the invention exhibit cytocidal activity that makes them well suited to therapeutic use in the prevention and/or treatment of cancer in the manner described in this specification.

### Persistence of Cells of the Invention

Persistence in vivo, and particularly within a subject, of cells exerting a therapeutic effect is important for effective prevention and/or treatment of diseases.

Cells of the invention, comprising nucleic acid constructs of the invention, exhibit increased persistence in vivo. This increased persistence in vivo, which is demonstrated in the Examples, represents a mechanism by which the therapeutic effects of the cells of the invention can be prolonged, and so their therapeutic utility increased, as compared to prior art cells.

By persistence it is meant that the cells of the invention remain viable for longer than suitable comparator CAR T-cells , such as unmodified CAR T-cells. Suitably the cells of the invention remain viable for longer in an in vivo environment than suitable comparator CAR T-cells, such as unmodified CAR T-cells. Suitably the cells of the invention remain viable for periods of time as defined hereinbelow.

Persistence of cells of the invention, or suitable comparator CAR T-cells, may be assessed experimentally in a number of different ways. Merely by way of example cell persistence may be defined with reference to the percentage of cells originally administered that remain viable within a recipient after a given period of time. It will be appreciated that a useful comparison between two or more populations of cells (such as a population of cells of the invention and a population of suitable comparator CAR T-cells, such as unmodified CAR T-cells) may be made after any given period of time, so long as the time elapsed is approximately the same for each of the populations of cells.

The skilled person will be aware of many suitable assays by which the persistence of a cell of the invention, or suitable comparator CAR T cell (such as an unmodified CAR T-Cell) may be assessed. Merely by way of example suitable assays are described in the Examples (point 1.3 and 5.1 of the examples), where they are used in the characterisation of exemplary cells of the invention.

Suitably, persistence may also be measured by the ability of the cells to exert cytocidal activity towards cells expressing a target protein recognised by the fusion target-binding protein after a given period of time. Suitable periods of time are defined below. Suitably viability or persistence may be determined or defined by cytocidal activity. Suitably the cells of the invention retain cytocidal activity towards cells expressing a target protein recognised by the fusion target-binding protein for longer than suitable comparator CAR T-cells, such as unmodified CAR T-cells. Suitably the cells of the invention retain cytocidal activity towards cells expressing a target protein recognised by the fusion target-binding protein for longer in an in vivo environment than suitable comparator CAR T-cells, such as unmodified CAR T-cells.

Suitably the cells of the invention demonstrate cytocidal activity towards cells expressing a target protein recognised by the fusion target-binding protein over at least any of the periods of time described below.

Suitably the cells of the invention also retain cytocidal activity upon repeated exposure to cells expressing a target protein recognised by the fusion target-binding protein. Suitably upon repeated exposure to cells expressing a target protein recognised by the fusion target-binding protein over a given period of time. Suitable periods of time are defined below. Suitably upon exposure to cells expressing a target protein recognised by the fusion target-binding protein at 48 hours, 96 hours, 128 hours and/or 148 hours, for example.

Cells of the invention may retain cytocidal activity in a recipient for a longer period than suitable comparator CAR T-cells. Suitably, cells of the invention may retain cytocidal activity in the recipient for at least 12 hours, at least 24 hours, at least 36 hours, at least 48 hours, at least 60 hours, at least 72 hours, at least 84 hours, at least 96 hours, at least 108 hours, at least 120 hours, at least 132 hours, at least 144 hours, at least 156 hours or more in a recipient. Suitably for at least 128 hours.

Suitably, cells of the invention may retain cytocidal activity in in the recipient for at least 5 days, at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 90 days, at least 95 days, or at least 100 days or more in a recipient. Suitably, a cell of the invention, comprising a protein of the invention, may remain viable for at least 150 days, at least 200 days, at least 250 days, at least 300 days, or at least 350 days or more in a recipient. Suitably a cell of the invention comprising a protein of the invention, may remain viable for at least 6 months, at least 9 months, at least 12 months at least 15 months, at least 18 months, at least 21 months, at least 24 months or more in the recipient. Suitably a cell of the invention comprising a protein of the invention, may remain viable for at least 1 year, at least 2 years at least 3 years, at least 4 years, at least 5 years, at least 6 years, at least 7 years, at least 8 years, at least 9 years or at least 10 years or more in the recipient. Suitable, the cell of the invention comprising the protein of the invention, may remain viable for at least years, for at least 15 years, for at least 20 years, for at least 25 years, for at least 30 years, for at least 35 years, for at least 40 years, for at least 45 years, for at least 50 years or more in the recipient.

Therefore ‘persisting’ as referred to below may refer to proportion of remaining viable cells, and/or may refer to the proportion of cells having cytocidal activity in a given population of cells, such as a population of cells of the invention.

The proportion of cells of the invention persisting after a set period of time may be at least 5% higher than that of suitable comparator CAR T-cells. Indeed, the proportion of cells of the invention persisting may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% higher than that of suitable comparator T-cells. The proportion of cells of the invention persisting after a set period of time may be at least 100%, or more, higher than that of suitable comparator CAR T-cells.

The proportion of cells of the invention persisting after a set period of time may be up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, or even up to 100% of the total number of cells of the invention originally administered.

Cells of the invention may persist in a recipient for a longer period than do suitable comparator CAR T-cells. Cells of the invention may persist in the recipient for up to 5% longer than a suitable comparator CAR T-cell. Cells of the invention may persist in the recipient for up to 10% longer, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, or even up to 100% longer than a suitable comparator T-cell.

Another way in which persistence of cells, such as cells of the invention, may be assessed is with reference to the length of time a cell remains viable in a recipient. Suitably a cell of the invention, comprising a protein of the invention, may remain viable for at least 5 days, at least days, at least 15 days, at least 20 days, at least 25 days, at least 30 days, at least 35 days, at least 40 days, at least 45 days, at least 50 days, at least 55 days, at least 60 days, at least days, at least 70 days, at least 75 days, at least 80 days, at least 85 days, at least 90 days, at least 95 days, or at least 100 days or more in a recipient. Suitably, a cell of the invention, comprising a protein of the invention, may remain viable for at least 150 days, at least 200 days, at least 250 days, at least 300 days, or at least 350 days or more in a recipient. Suitably a cell of the invention comprising a protein of the invention, may remain viable for at least 6 months, at least 9 months, at least 12 months at least 15 months, at least 18 months, at least 21 months, at least 24 months or more in the recipient. Suitably a cell of the invention comprising a protein of the invention, may remain viable for at least 1 year, at least 2 years at least 3 years, at least 4 years, at least 5 years, at least 6 years, at least 7 years, at least 8 years, at least 9 years or at least 10 years or more in the recipient. Suitable, the cell of the invention comprising the protein of the invention, may remain viable for at least 10 years, for at least 15 years, for at least 20 years, for at least 25 years, for at least 30 years, for at least years, for at least 40 years, for at least 45 years, for at least 50 years or more in the recipient.

### Proliferation of Cells of the Invention

Activation of cells of the invention, via binding of the fusion target-binding protein to the corresponding target protein , induces cell proliferation. This allows the production of increased numbers of cells able to exert a therapeutic activity. However, such cell proliferation is normally inhibited in the tumour microenvironment, and this has contributed to the failure of CAR T cell treatments disclosed in the prior art.

The cells of the invention demonstrate a marked increase in proliferation, especially in response to contact with cancer cells expressing the target protein recognised by the fusion target-binding protein expressed by the cells of the invention. Since cell proliferation results in expansion of populations of cells of the invention that are then able to exert their therapeutic cytocidal activity, this is highly advantageous. Suitably therefore, the cells demonstrate increased proliferation in the presence of cancer cells expressing a target protein recognised by the fusion target-binding protein, compared to comparator CAR T cells.

Proliferation of cells, such as cells of the invention may be assessed experimentally in a number of ways know by a person skilled in the art. Merely by way of example cell proliferation may be defined with reference to comparison between two or more populations of cells (such as a population of cells of the invention and a population of suitable comparator CAR T-cells, such as unmodified CAR T cells). Comparisons may be made after any given period of time, so long as the time elapsed is approximately the same for each of the populations of cells.

The skilled person will be aware of many suitable assays by which the proliferation of a cell of the invention, or suitable comparator CAR T cell (such as an unmodified CAR T-Cell) may be assessed. Merely by way of example suitable assays are described in the Examples (point 2.3 of the examples), where they are used in the characterisation of exemplary cells of the invention.

In such conditions, the cells of the invention may exhibit a rate of proliferation that is at least 5% higher than that of suitable comparator CAR T-cells, at least 10% higher than that of suitable comparator T-cells, at least 15% higher than that of suitable comparator T-cells, at least 20% higher than that of suitable comparator T-cells, at least 25% higher than that of suitable comparator T-cells, at least 30% higher than that of suitable comparator T-cells, at least 35% higher than that of suitable comparator T-cells, at least 40% higher than that of suitable comparator T-cells, at least 45% higher than that of suitable comparator T-cells, at least 50% higher than that of suitable comparator T-cells, at least 55% higher than that of suitable comparator T-cells, at least 60% higher than that of suitable comparator T-cells, at least 65% higher than that of suitable comparator T-cells, at least 70% higher than that of suitable comparator T-cells, at least 75% higher than that of suitable comparator T-cells, at least 80% higher than that of suitable comparator T-cells, at least 85% higher than that of suitable comparator T-cells, at least 90% higher than that of suitable comparator T-cells, or at least 95% higher than that of suitable comparator T-cells. Indeed, the cells of the invention may exhibit a rate of proliferation that is at least 100%, or more, higher than that of suitable comparator T-cells in the same experimental conditions.

Alternatively, proliferation of cells may be assessed with reference to the number of cells present in a recipient after a set period of time from administration, as compared to the number of comparator CAR T-cells present under the same conditions. The number of cells of the invention present in a recipient after a given time may be at least 5% higher than that of suitable comparator T-cells, at least 10% higher than that of suitable comparator T-cells, at least 15% higher than that of suitable comparator T-cells, at least 20% higher than that of suitable comparator T-cells, at least 25% higher than that of suitable comparator T-cells, at least 30% higher than that of suitable comparator T-cells, at least 35% higher than that of suitable comparator T-cells, at least 40% higher than that of suitable comparator T-cells, at least 45% higher than that of suitable comparator T-cells, at least 50% higher than that of suitable comparator T-cells, at least 55% higher than that of suitable comparator T-cells, at least 60% higher than that of suitable comparator T-cells, at least 65% higher than that of suitable comparator T-cells, at least 70% higher than that of suitable comparator T-cells, at least 75% higher than that of suitable comparator T-cells, at least 80% higher than that of suitable comparator T-cells, at least 85% higher than that of suitable comparator T-cells, at least 90% higher than that of suitable comparator T-cells, or at least 95% higher than that of suitable comparator T-cells if both cells of the invention and comparator T-cells are administered in approximately equal amounts.

### Comparator/Control Cells

Cells of the invention may be assessed in a variety of ways known to the person skilled in the art. For the purposes of the present disclosure cells of the present invention may be compared to “comparator CAR T-cells”. Comparator CAR T cells are suitably unmodified CAR T cells, which may also be referred to as “unmodified CAR T cells” or “ control CAR T cells” herein. Suitably, comparator CAR T cells do not comprise, or have not been modified to comprise a sequence encoding an amino acid transporter such as SLC7A5 or SLC7A11 as defined herein. Suitably a comparator CAR T cell may be the same in all other respects as a cell of the invention, except that it does not comprise or express an amino acid transporter such as SLC7A5 or SLC7A11 as defined herein.

Therapeutic activity of the cells of the invention may be determined as compared to comparator CAR T-cells. Suitable comparator CAR T- cells would be easily determined by the skilled person. Suitably, a comparator CAR T-cell may be a T cell comprising a CAR that does not comprise an amino acid transporter such as either SLC7A5 or SLC7A11, or which has not been modified to comprise an amino acid transporter such as either SLC7A5 or SLC7A11. Suitably, a comparator CAR T-cell may be a CAR T cell expressing a CAR that does not express an amino acid transporter such as either SLC7A5 or SLC7A11. Suitably, a comparator CAR T-cell expressing a CAR may express any CAR as defined herein, for example an anti-GD2 CAR, or an anti-CD33 CAR. Suitably in comparisons, the comparator CAR T cell may comprise and express the same CAR as the cell of the invention.

The skilled person would have the relevant skills and knowledge to identify suitable comparator CAR T-cell from those known in the prior art in order to determine the therapeutic activity of the cells of the invention.

### Medical Uses and Methods of Treatment

The nucleic acid constructs of the invention, particularly when provided in the cells of the second aspect of the invention, are well suited to medical use, which is to say for use as medicaments in the prevention and/or treatment of diseases.

Prevention of a disease may be required when a subject has not yet developed a disease, but has been identified as being at risk of developing the disease in future. Suitably such identification may be based upon details such as the clinical history of the subject or their family, results of genetic testing of the subject of their family, or exposure risk to known disease causing agents. In the case of cancer, prevention may be desirable in the case of a subject exhibiting symptoms or features of pre-malignant disease.

Treatment of a disease may be required once a subject has been identified as already having developed a disease. The stage of development of the disease at the time of identification may be symptomatic or asymptomatic. Such identification may be based upon clinical assessment of the subject, symptoms presented by the subject, or analysis of samples provided by the subject (such biopsies, blood samples, or the like, allowing for the identification of the presence of malignancies, infectious agents, or other indicators of pathology).

Prevention and/or Treatment of Cancer

In particular, the cells, nucleic acid constructs, or pharmaceutical compositions of the invention may be of use in the prevention and/or treatment of cancer. It is in these applications that the increased cytocidal and proliferative activity of cells of the invention (as compared to control CAR-T cells) under arginine-depleted conditions representative of the tumour microenvironment are particularly advantageous.

Suitable examples of cancers that may be prevented and/or treated by medical uses or methods of treatment utilising the cells, nucleic acid constructs, or pharmaceutical compositions of the invention include those associated with cancer cell expression of one or more markers selected from the group consisting of: GD2; CD33; Mesothelin; EGFRvIII; VEGFR2; FAP; EpCam; GPC3; CD133; IL13Ra; EphA2; Muc1; BCMA; CD70; CD123; ROR1; PSMA; CD5; GAP; CEA; PSCA; Her2; and CD19. Such cancers may be treated by use of fusion target-binding proteins incorporating corresponding target binding moieties.

Merely by way of example, suitable cancers that may be prevented and/or treated by medical uses or methods of treatment of the invention may be selected from the group consisting of:

neuroblastoma; mesothelioma; ovarian cancer; breast cancer; colon cancer; medulloblastoma; pancreatic cancer; prostate cancer; testicular cancer; acute myeloid leukaemia; glioblastoma; osteosarcoma; and melanoma.

### Pharmaceutical Compositions and Formulations

The present invention also provides compositions including cells, or nucleic acid constructs of the invention. In particular, the invention provides pharmaceutical compositions and formulations, such as unit dose form compositions including cells or nucleic adds of the invention for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the choice of carrier is determined in part by the particular protein, cell, or nucleic acid of the invention, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001 to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin ; gelatine, or immunoglobulins; hydrophilic polymers such as polyvinylpyrrolidone; amino acids such as glycine; glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes): and/or non-ionic surfactants such as polyethylene glycol (PEG).

Buffering agents are included in some embodiments of the compositions of the invention. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001 to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the proteins s, cells, or nucleic acids of the invention, preferably those with activities complementary to the proteins s, cells, or nucleic acids of the invention ; where the respective activities do not adversely affect one another. Such active ingredients are suitably present in combination in amounts that are effective for the purpose intended. Thus ; in some embodiments ; the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the CARs, cells, or nucleic acids of the invention in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the proteins, cells, or nucleic acids of the invention, by multiple bolus administrations of the proteins, cells ; or nucleic acids, or by continuous infusion administration of the proteins, cells, or nucleic acids.

The compositions may be administered using standard administration techniques ; formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the proteins, cells, or nucleic acids of the invention in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavouring agents, and/or colours, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminium monostearate and gelatine.

The formulations to be used for in vivo administration are generally sterile. Sterility may be readily accomplished, e.g., by filtration through sterile filtration membranes.

### Doses and Dosage Regimens

### Size or Amount of Doses

The cells or nucleic acids of the invention may be provided in a first dose, and optionally in subsequent doses. In some embodiments, the first or subsequent dose contains a number of proteins, cells, or nucleic acids of the invention in the range from about 10⁵ to about 10⁶ of such cells per kilogram body weight of the subject, and/or a number of such cells that is no more than about 10⁵ or about 10⁶ such cells per kilogram body weight of the subject. For example, in some embodiments, the first or subsequent dose includes less than or no more than at or about 1×10⁵, at or about 2×10⁵, at or about 5×10⁵, or at or about 1×10⁶ of such cells per kilogram body weight of the subject. In some embodiments, the first dose includes at or about 1×10⁵, at or about 2×10⁵, at or about 5×10⁵, or at or about 1×10⁶ of such cells per kilogram body weight of the subject, or a value within the range between any two of the foregoing values.

In some embodiments, for example, where the subject is a human, the first or subsequent dose includes fewer than about 1×10total cells or nucleic acids of the invention e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the first or subsequent dose contains fewer than about 1×10⁸ total cells or nucleic acids of the invention per m² of the subject, e.g., in the range of about 1×10⁶ to 1×10 8 such cells per m 2 of the subject, such as 2×106, 5×106, 1×107, 5×107, or 1×108 such cells per m of the subject, or the range between any two of the foregoing values.

In certain embodiments, the number of cells or nucleic acids of the invention in the first or subsequent dose is greater than about 1×10⁶ such cells or nucleic acids of the invention per kilogram body weight of the subject, e.g., 2×10⁶, 3×10⁶, 5×10⁶, 1×10⁷, 5×10⁷, 1×10⁶, 1×10⁹, or 1×10¹⁰ such cells per kilogram of body weight and/or, 1×108, or 1×109, 1×1010 such cells per m 2 of the subject or total, or the range between any two of the foregoing values.

In some embodiments, the number of cells or nucleic acids of the invention administered in the subsequent dose is the same as or similar to the number of proteins, cells, or nucleic acids of the invention administered in the first dose in any of the embodiments herein, such as less than or no more than at or about 1×10⁵, at or about 2×10⁵, at or about 5×10⁵, or at or about 1×10⁶ of such cells per kilogram body weight of the subject. In some embodiments, the subsequent dose(s) contains at or about 1×10⁵, at or about 2×10⁵, at or about 5×10⁵, or at or about 1×10⁶ of such cells per kilogram body weight of the subject, or a value within the range between any two of the foregoing values. In some embodiments, such values refer to numbers of cells or nucleic acids of the invention. In some aspects, the subsequent dose is larger than the first dose. For example, in some embodiments, the subsequent dose contains more than about 1×10⁶ cells or nucleic acids of the invention per kilogram body weight of the subject, such as about 3×10⁶, 5×10⁶, 1×107, 1×108, or 1×109 such cells per kilogram body weight of the subject, In some embodiments, the amount or size of the subsequent dose is sufficient to reduce disease burden or an indicator thereof, and/or one or more symptoms of the disease or condition. In some embodiments, the second (or other subsequent) dose is of a size effective to improve survival of the subject, for example, to induce survival, relapse-free survival, or event-free survival of the subject for at least 6 months, or at least 1, 2, 3, 4, or 5 years. In some embodiments, the number of cells or nucleic acids of the invention administered and/or number of such cells administered per body weight of the subject in the subsequent dose is at least 2-fold, 5-fold, 10-fold, 50-fold, or 100-fold or more greater than the number administered in the first dose. In some embodiments, disease burden, tumour size, tumour volume, tumour mass, and/or tumour load or bulk is reduced following the subsequent dose by at least at or about 50, 60, 70, 80, 90% or more compared to that immediately prior to the administration of the first dose or of the second (or other subsequent) dose.

In other embodiments, the number of cells or nucleic acids of the invention administered in the subsequent dose is lower than the number of cells or nucleic acids of the invention administered in the first dose.

In some embodiments, multiple subsequent doses are administered following the first dose, such that an additional dose or doses are administered following administration of the second (or other subsequent) dose, In some aspects, the number of cells administered to the subject in the additional subsequent dose or doses (i.e., the third, fourth, fifth, and so forth) is the same as or similar to the first dose, the second dose, and/or other subsequent dose. In some embodiments, the additional dose or doses are larger than prior doses.

In some aspects, the size of the first and/or subsequent dose is determined based on one or more criteria such as response of the subject to prior treatment, e.g. chemotherapy, disease burden in the subject, such as tumour load, bulk, size, or degree, extent, or type of metastasis, stage, and/or likelihood or incidence of the subject developing toxic outcomes, e.g., CRS, macrophage activation syndrome, tumour lysis syndrome, neurotoxicity, and/or a host immune response against the cells and/or recombinant receptors being administered.

In some aspects, the size of the first and/or subsequent dose is determined by the burden of the disease or condition in the subject, For example, in some aspects, the number of proteins, cells, or nucleic acids of the invention administered in the first dose is determined based on the tumour burden that is present in the subject immediately prior to administration of the first dose. In some embodiments, the size of the first and/or subsequent dose is inversely correlated with disease burden. In some aspects, as in the context of a large disease burden, the subject is administered a low number of proteins, cells, or nucleic acids of the invention, for example less than about 1×10⁶ cells or nucleic acids of the invention per kilogram of body weight of the subject. In other embodiments, as in the context of a lower disease burden, the subject is administered a larger number of cells or nucleic acids of the invention, such as more than about 1×10⁶ cells or nucleic acids of the invention per kilogram body weight of the subject.

In some aspects, the number of cells or nucleic acids of the invention administered in the subsequent dose is determined based on the tumour burden that is present in the subject following administration of the first dose. In some embodiments, e.g. where the first dose has decreased disease burden or has done so below a particular threshold amount or level, e.g., one above which there is an increased risk of toxic outcome, the subsequent dose is large, e.g. more than 1×10⁶ cells or nucleic acids of the invention per kilogram body weight, and/or is larger than the first dose. In other aspects, the number of cells or nucleic acids of the invention administered in the subsequent dose is low, e.g. less than about 1×10⁶, e.g. the same as or lower than the first dose, where the first dose has reduced tumour burden to a small extent or where the first dose has not led to a detectable reduction in tumour burden.

In some embodiments, the number of cells or nucleic acids of the invention administered in the first dose is lower than the number of cells or nucleic acids of the invention administered in other methods, such as those in which a large single dose of cells is administered, such as to administer the cells or nucleic acids of the invention in before an immune response develops. Thus, in some embodiments, the methods reduce toxicity or toxic outcomes as compared to other methods that involve administration of a larger dose.

In some embodiments, the first dose includes the cells or nucleic acids of the invention in an amount that does not cause or reduces the likelihood of toxicity or toxic outcomes, such as cytokine release syndrome (CRS), severe CRS (sCRS), macrophage activation syndrome, tumour lysis syndrome, fever of at least at or about 38 degrees Celsius for three or more days and a plasma level of CRP of at least at or about 20 mg/dL, and/or neurotoxicity. In some aspects, the number of cells administered in the first dose is determined based on the likelihood that the subject will exhibit toxicity or toxic outcomes, such as CRS, sCRS, and/or CRS-related outcomes following administration of the cells. For example, in some embodiments, the likelihood for the development of toxic outcomes in a subject is predicted based on tumour burden. In some embodiments, the methods include detecting or assessing the toxic outcome and/or disease burden prior to the administration of the dose.

In some embodiments, the second (or other subsequent) dose is administered at a time point at which a clinical risk for developing cytokine-release syndrome (CRS), macrophage activation syndrome, or tumour lysis syndrome, or neurotoxicity is not present or has passed or has subsided following the first administration, such as after a critical window after which such events generally have subsided and/or are less likely to occur, e.g., in 60, 70, 80, 90, or 95% of subjects with a particular disease or condition.

In some embodiments the dose of the cells of the invention is lower than previous doses used for such therapies. Advantageously, the increased proliferation of the cells of the invention and the persistence of the cells of the invention allows for a lower dose of cells of the invention to be used in therapy.

### Timing of Doses

In some aspects, the timing of the second or subsequent dose is measured from the initiation of the first dose to the initiation of the subsequent dose. In other embodiments, the timing of the subsequent dose is measured from the completion of the first dose, or from the median day of administration of the first dose, e.g. in the context of split dosing, described herein, where a dose is administered over more than one day, e.g. over 2 days or over 3 days.

In some embodiments, whether a subsequent dose of proteins, cells, or nucleic acids of the invention distinct from that of the first dose is administered, is determined based on the presence or degree of an immune response or detectable immune response in the subject to the proteins, cells, or nucleic acids of the invention of the first dose. In some aspects, a subsequent dose containing cells expressing a different receptor than the cells of the first dose will be administered to a subject with a detectable host adaptive immune response, or an immune response that has become established or reached a certain level, stage, or degree.

In some embodiments, the second (or other subsequent) dose is administered at a point n time at which a second administration of proteins, cells, or nucleic acids of the invention is likely to be or is predicted to be eliminated by the host immune system. The likeliness of developing an immune response may be determined by measuring receptor-specific immune responses in the subject following administration of the first dose, as described herein.

For example, in some embodiments, subjects may be tested following the first (or other prior) dose and prior to the second (or other subsequent) dose to determine whether an immune response is detectable in the subject after the first dose. In some such embodiments, the detection of an immune response to the first dose may trigger the need to administer the second dose.

In some aspects, samples from the subjects may be tested to determine if there is a decline in or lower than desired exposure, for example, less than a certain number or concentration of cells, as described herein, in the subject after the first or prior dose. In some such aspects, the detection of a decline in the exposure of the subject to the cells may trigger the need to administer the second dose.

In some embodiments, the subsequent dose is administered at a point in time at which the disease or condition in the subject has not relapsed following the reduction in disease burden in response to the first or prior dose. In some embodiments, the disease burden reduction is indicated by a reduction in one or more factors, such as load or number of disease cells in the subject or fluid or organ or tissue thereof, the mass or volume of a tumour, or the degree or extent of metastases. Such a factor is deemed to have relapsed if after reduction in the factor in response to an initial treatment or administration, the factor subsequently increases.

In some embodiments, the second dose is administered at a point in time at which the disease has relapsed. In some embodiments, the relapse is in one or one or more factors, or in the disease burden generally. In some aspects, the subsequent dose is administered at a point in time at which the subject, disease burden, or factor thereof has relapsed as compared to the lowest point measured or reached following the first or prior administration, but still is lower compared to the time immediately prior to the first dose. In some embodiments, the subject is administered the subsequent dose at a point in time at which disease burden or factor indicative thereof has not changed, e.g. at a time when an increase in disease burden has been prevented.

In some embodiments, the subsequent dose is administered at a time when a host adaptive immune response is detected, has become established, or has reached a certain level, degree, or stage. In some aspects, the subsequent dose is administered following the development of a memory immune response in the subject.

In some aspects, the time between the administration of the first dose and the administration of the subsequent dose is about 28 to about 35 days, about 29 to about 35 days, or more than about 35 days. In some embodiments, the administration of the second dose is at a time point more than about 28 days after the administration of the first dose. In some aspects, the time between the first and subsequent dose is about 28 days.

In some embodiments, an additional dose or doses, e.g. subsequent doses, are administered following administration of the second dose. In some aspects, the additional dose or doses are administered at least about 28 days following administration of a prior dose. In some embodiments, no dose is administered less than about 28 days following the prior dose.

In some embodiments, e.g. where one or more consecutive doses are administered to the subject, the consecutive doses may be separated by about 7, about 14, about 15, about 21, about 27, or about 28 days. In some aspects, the consecutive dose is administered 21 days following a prior dose. In some embodiments, the consecutive dose is administered between 14 and 28 days following administration of a prior dose.

In any of the embodiments, the methods in some cases include the administration of the first or prior dose and the subsequent dose(s), and in other cases include the administration of the subsequent dose(s) to a subject who has previously received the first or prior dose but do not include the administration of the first or prior dose itself. Thus, the methods in some cases involve the administration of consolidating treatment, such as by administering a consolidating subsequent dose to a subject that has previously received a dose of proteins, cells, or nucleic acids of the invention.

In some embodiments, disease burden, tumor size, tumor volume, tumor mass, and/or tumor load or bulk is reduced following the subsequent dose by at least at or about 50, 60, 70, 80, 90% or more compared to that immediately prior to the administration of the first or prior dose or of the second or subsequent dose.

In some embodiments the cells of the invention are administered less frequently than previous dosage schedules used for such therapies. Advantageously, the increased proliferation of the cells of the invention and the persistence of the cells of the invention allows for fewer doses of cells of the invention, and less frequent doses, to be used in therapy.

The invention will now be further described with reference to the following Examples.

### EXAMPLES

Various studies investigating the manufacture and characterisation of cells of the invention (produced by transduction with nucleic acid constructs of the invention) have been undertaken by the inventors. Details of the results of these studies are set out below, and experimental protocols used in the studies are set out at the end of the Examples.

1.1 Nucleic Acid Constructs of the Invention Can be Transduced into Human T Cells

T cells from two exemplary donors were subjected to one of the following protocols:
mock transduction;
transduction with a nucleic acid encoding an unmodified anti-CD33 CAR;
transduction with a nucleic acid construct of the invention encoding an anti-CD33 CAR and amino acid transporter SLC7A5; or
transduction with a nucleic acid construct of the invention encoding an anti-CD33 CAR and amino acid transporter SLC7A11.

Transduction was accomplished using either retroviral or lentiviral vectors incorporating nucleic acid constructs of the invention (SEQ ID NO: 20 or SEQ ID NO: 22) that encode an anti-CD33 CAR and the amino acid transporter SLC7A5 or SLC7A11 (respectively).

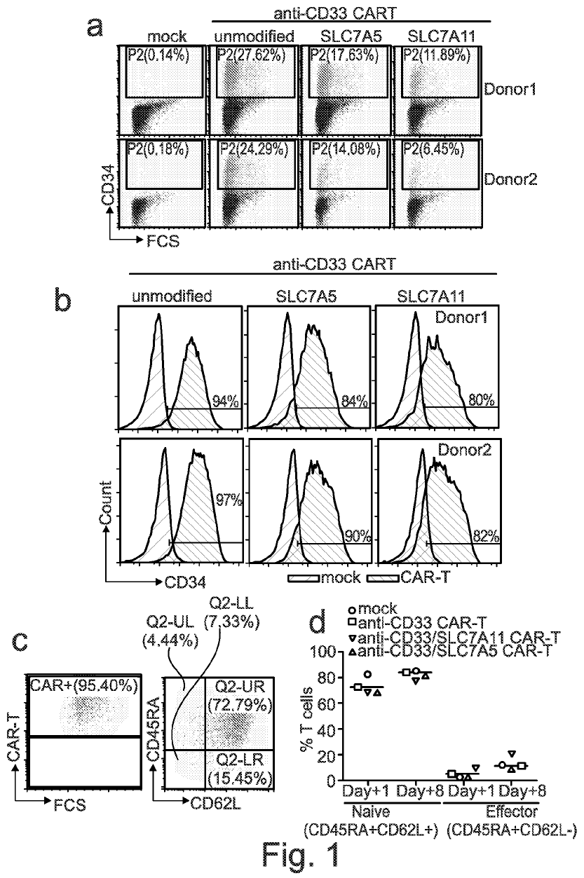

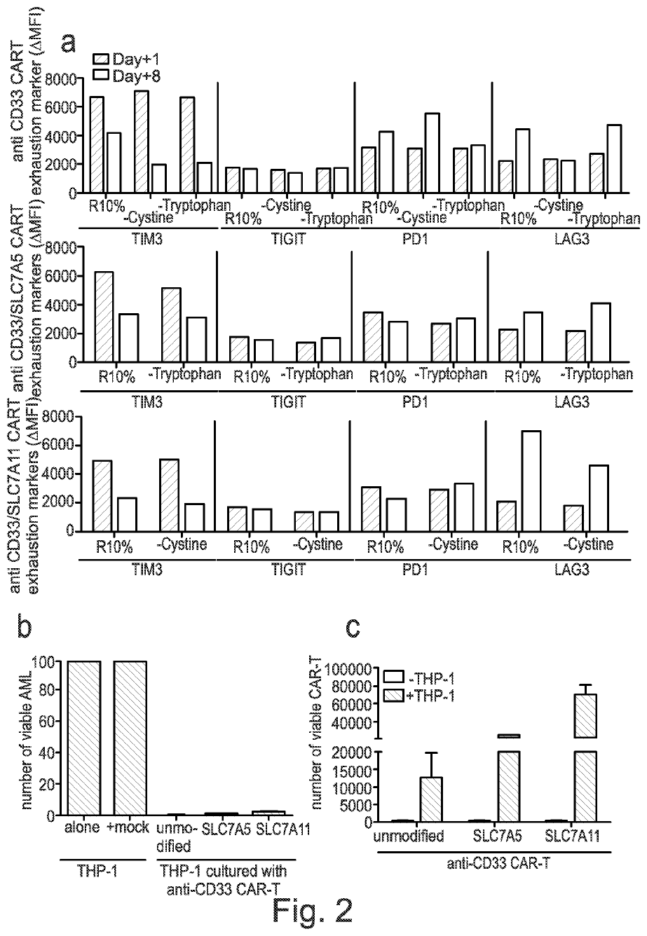

Panel “a” of FIG. 1 demonstrates effective transduction using retroviral vectors, as assessed by measuring expression of a truncated form of CD34 (tCD34) using flow cytometry. Cells in the upper region of each plot (outlined by a rectangle) indicate successful transduction leading to cell expression. As can be seen, both nucleic acid constructs of the invention were capable of successful transduction.

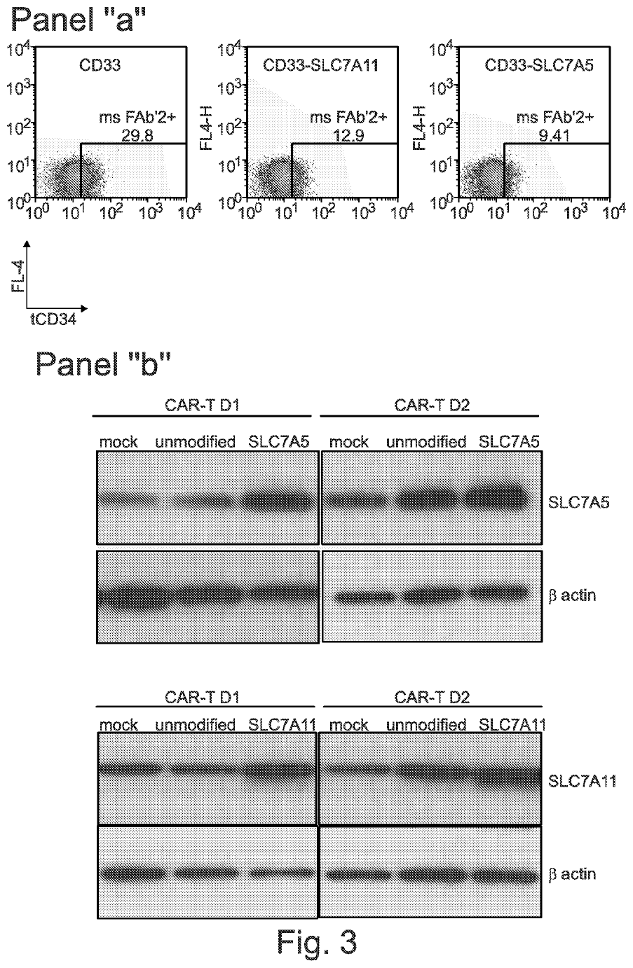

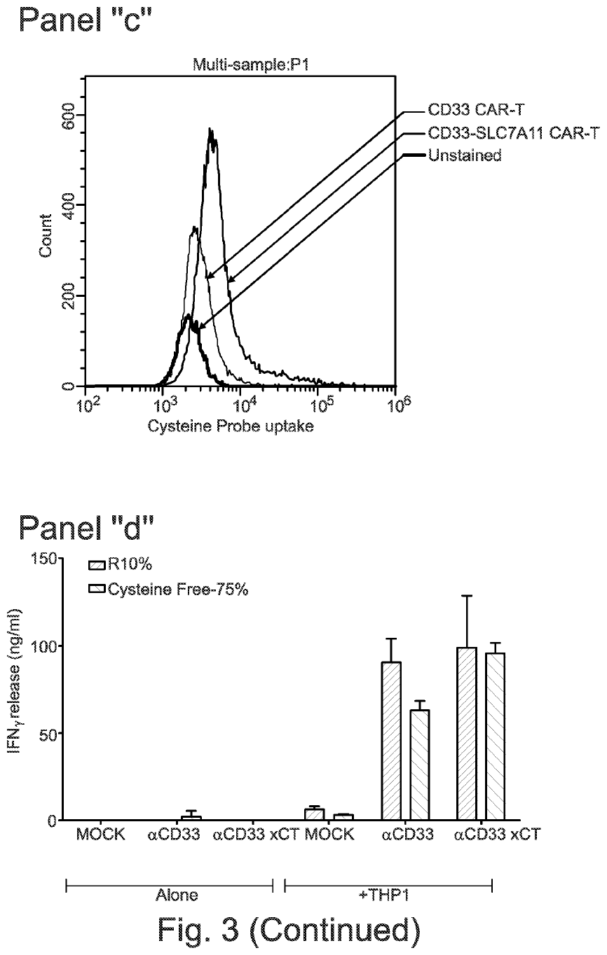

Panel “a” of FIG. 3 demonstrates effective transduction using lentiviral vectors, setting out the results of flow cytometry prior to cell sorting of the populations of cells produced. As can be seen transduction with a nucleic acid construct of the invention encoding an anti-CD33 CAR in combination with either SLC7A11 or SLC7A5 is comparably effective to transduction using a nucleic acid encoding the anti-CD33 CAR alone.

1.2 Cells of the Invention can be Sorted to a High Degree of Purity on the Basis of tCD34 Expression

Panel “b” of FIG. 1 illustrates the results of a study in which T cells from two exemplary donors were either:
transduced with a nucleic acid encoding an unmodified anti-CD33 CAR;
transduced with a nucleic acid construct of the invention encoding an anti-CD33 CAR and amino acid transporter SLC7A5; or
transduced with a nucleic acid construct of the invention encoding an anti-CD33 CAR and amino acid transporter SLC7A11.

Cells then underwent cell sorting on the basis of tCD34 expression. As can be seen, all cells produced (control “unmodified” cells, and the two variants of cells of the invention: “SLC7A5” and “SLC7A11”) were able to be sorted to a high degree of purity on the basis of tCD34 expression.

1.3 Transduction with Nucleic Acid Constructs of the Invention Does Not Adversely Impact Numbers of Naïve or Effector T Cell Phenotype

Panels “c” and “d” of FIG. 1 set out the results of a study investigating the impact of transduction with (and expression of) nucleic acid constructs of the invention on numbers of cells having naïve or effector T cell phenotypes among the resulting population of cells of the invention.

Naïve T cells were characterised as CD45RA⁺/CD62L⁺, while effector T cells were characterised as CD45RAV⁺/CD62L⁻.

Cells from two representative donors underwent:
mock transduction;
transduction with a nucleic acid encoding an unmodified anti-CD33 CAR;
transduction with a nucleic acid construct of the invention encoding an anti-CD33 CAR and amino acid transporter SLC7A5; or
transduction with a nucleic acid construct of the invention encoding an anti-CD33 CAR and amino acid transporter SLC7A11.

The resultant cell populations were then labelled for CD45RA and CD62L, before undergoing flow cytometry to investigate expression of these markers. As can be seen from the results set out in panels “c” and “d” of FIG. 1, the proportion of cells of either the naïve or effector T cell phenotype was not significantly different between populations of cells of the invention or populations of relevant control cells.

2.1 Transduction with Nucleic Acid Constructs of the Invention Does Not Increase Cell Exhaustion

The inventors undertook a study to investigate the effects of transduction with (and expression of) nucleic acid constructs of the invention on markers of T cell exhaustion. The results of this study are set out in panel “a” of FIG. 2.

The levels of expression of markers of T cell exhaustion were compared between control cells (expressing an unmodified anti-CD33 CAR) and cells of the invention (expressing an anti-CD33 and either SLC7A5 or SLC7A11). Markers were assessed by flow cytometry after culture of CAR T cells in “normal” cell culture conditions (“R10%”—RPMI medium supplemented with 10% foetal bovine serum) and low amino acid conditions (specifically, culture in reduced concentration of cystine or reduced concentration of tryptophan).

As can be seen, the results demonstrate that incorporation of the nucleic acid constructs of the invention has no detrimental effect on T cell exhaustion.

### 2.2 Cells of the Invention Retain Cytocidal Activity

The inventors investigated the ability of cells of the invention to kill cells expressing suitable target molecules. For this study THP-1 cells (cells of a cancer cell line derived from an acute myeloid leukaemia patient) were chosen for their expression of CD33 target molecules.

To produce cell populations to be investigated, cells underwent:
mock transduction;
transduction with a nucleic acid encoding an unmodified anti-CD33 CAR;
transduction with a nucleic acid construct of the invention encoding an anti-CD33 CAR and amino acid transporter SLC7A5; or
transduction with a nucleic acid construct of the invention encoding an anti-CD33 CAR and amino acid transporter SLC7A11.

The resultant cell populations were cultured with THP-1 cells for 7 days, at the end of which the percentage of viable THP-1 cells remaining was calculated.

Culture with THP-1 cells in this manner replicates physiological conditions observed in cancer, in that the AML cells deplete amino acids within the culture medium. Accordingly, this in vitro model provides a useful indication of the likelihood that a putative therapeutic agent will give beneficial results in vivo.

The results set out in panel “b” of FIG. 2 illustrate that, when assessed in this model, cells of the invention (comprising nucleic acid construct encoding an anti-CD33 fusion target-binding protein and either the SLC7A5 or SLC7A11 amino acid transporter) retain two biological functions essential for therapeutic effectiveness. First, the presence of the sequence encoding an amino acid transporter does not have any adverse impact on their specificity for cancer cells expressing CD33. Secondly, the cells of the invention retain cytocidal activity, and are as effective in killing cancer cells expressing CD33 as are T cells transduced to express an unmodified anti-CD33 CAR. These results thus provide a useful indication that cells of the invention will be useful in the treatment of cancer in vivo.

### 2.3 Cells of the Invention Exhibit Increased Proliferation in Response to Cells Expressing Target Molecules

Panel “c” of FIG. 2 illustrates the results of a study to investigate the response of cells of the invention to the presence of cells expressing a target molecule (specifically, cells of the THP-1 cancer cell line expressing CD33).

T cells expressing either an anti-CD33 CAR (designated “unmodified” cells), or expressing an anti-CD33 CAR and one of either SLC7A5 or SLC7A11, were cultured for 7 days in the presence or absence of THP-1 cells. As noted above, culture with AML blasts in this manner constitutes a useful model of physiological conditions associated with cancer in vivo, in both the presence of cancer cells (and the associated target molecules that they express) and generation of an environment in which amino acid concentrations are depleted.

The results shown demonstrate that the size of the cell populations did not change in the absence of stimulation by CD33+cancer cells. However, when cancer cells are present, cells of the invention are stimulated to proliferate strongly. The increased proliferation stimulated in the cells of the invention is markedly elevated as compared to that observed in cells expressing “unmodified” CARs (i.e. CARs alone without expression of additional amino acid transporters).

Cell proliferation is most increased in cells of the invention comprising SLC7A11, but both sets of cells of cells of the invention (those transduced with nucleic acid constructs encoding a CAR and SLC7A11 and those transduced with constructs encoding a CAR and SLC7A5) demonstrate elevated proliferation as compared to the appropriate controls, indicating that both offer favourable properties for therapeutic use.

3.1 Transduction with Nucleic Acid Constructs of the Invention Increases Cellular Expression of Amino Acid Transporters

Panel “b” of FIG. 3 sets out the results of Western blotting to investigate expression of amino add transporters SLC7A5 or SLC7A11 by T cells from two exemplary donors that have undergone:
mock transduction;
transduction with a nucleic acid encoding an unmodified anti-CD33 CAR;
transduction with a nucleic acid construct of the invention encoding an anti-CD33 CAR and amino acid transporter SLC7A5; or transduction with a nucleic acid construct of the invention encoding an anti-CD33 CAR and amino acid transporter SLC7A11.

Western blotting was conducted using antibodies reactive to SLC7A5 or SLC7A11, or to 13 actin (as a control).

The results demonstrate that cells of the invention, produced by transduction with nucleic acid constructs of the invention, exhibit levels of expression of amino acid transporters encoded by the nucleic acid constructs that are elevated as compared to expression by cells that underwent mock transduction, or transduction to express an unmodified CAR.

### 3.2 Cells of the Invention Exhibit Increased Uptake of Amino Acids

Cysteine uptake was compared between cells of the invention (comprising a nucleic acid construct encoding an anti-CD33 CAR and the amino acid transporter SLC7A11), and control cells expressing the CAR (but not SLC7A11). Uptake was measured by assessing levels of a fluorescently labelled cysteine probe using flow cytometry, and the results are set out in panel “c” of FIG. 3.

Here it can be seen that the staining observed in respect of cells of the invention is notably higher than that seen for the control cells expressing CARs alone. This indicates that the results observed in respect of increased expression of amino acid transporters are reflected in increased biological activity of such transporters in the cells of the invention.

### 3.2 Cells of the Invention Exhibit Increased Activation in Conditions of Depleted Amino Acid Concentrations

The inventors investigated the activation response of cells of the invention to the presence of cancer cells. This was investigated in “normal” cell culture conditions (RPMI medium supplemented with 10% foetal bovine serum) and in conditions of depleted amino acid concentration (75% cysteine free) representing the conditions observed in cancer in vivo.

Activation of the cells of the invention was compared to the response of cells that had undergone mock transduction, and to that of cells transduced with an anti-CD33 CAR, but no additional amino acid transporter. Activation was assessed by investigation of interferon-γ release, and the results are shown in panel “d” of FIG. 3.

It can be seen that none of the T cells (“mock”—mock transfected; “aCD33”—the CAR only; or “aCD33 xCT”—cells of the invention with an anti-CD33 CAR and SLC7A11) exhibited significant IFNγ release (indicative of activation) in the absence of THP-1 cancer cells (“alone” results on the left hand side of the panel).

IFNγ release was observed in response to the presence of THP-1 cells. In normal cell culture conditions, the activation observed in respect of the cells of the invention was slightly increased, as compared to control CAR T cells. In conditions of depleted cysteine representative of cancer in vivo, the activation of control CAR T cells decreased significantly, illustrative of the immunosuppressive effects of these conditions. In contrast, the activation of cells of the invention was hardly altered by the reduction in cysteine concentration, yielding a significantly improved response as compared to control CAR T cells in the same conditions. This indicates that the cells of the invention are better able to survive and function in otherwise immunosuppressive conditions associated with cancer.

### 4.1 Unmodified T Cells and CAR T Cells Have Impaired Proliferation and Viability in Amino Acid Depleted Culture Conditions

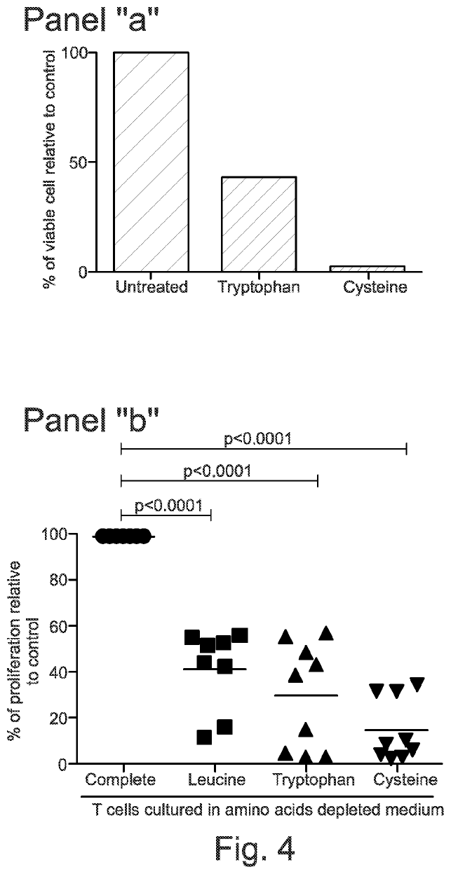

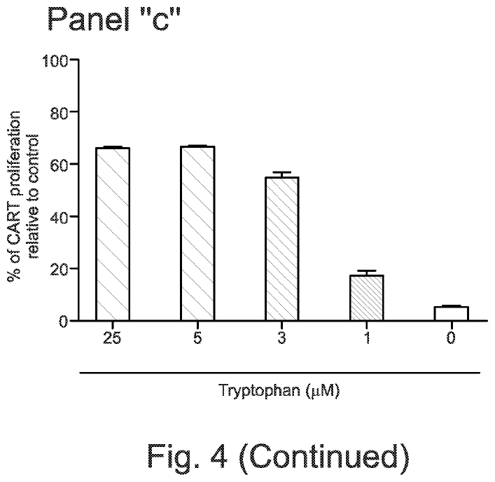

The immunosuppressive effects of cancer are well known. The results set out in panels “a”, “b” and “c” of FIG. 4 illustrate that reduced concentration of amino acids contribute to poor viability of activated T cells and CAR T cells in vitro.

As shown in panel “a” proliferation of healthy donor T cells (stimulated by treatment with anti-CD3/CD28 antibodies) is inhibited in media depleted in respect of leucine, tryptophan, or cysteine. Proliferation was measured by 3 H-thymidine assay after 72 hours of culture.

Panel “b” shows that viability of CAR T cells (not incorporating additional amino acid transporters, and hence not constituting cells of the invention) is impaired when cultured in tryptophan-depleted or cysteine-depleted media.

The results in panel “c” demonstrate that proliferation of stimulated control CAR T cells is reduced in proportion to a reduction in tryptophan concentration in culture.

### 5.1 Modified CAR-T Cells Maintain The Capacity to Kill Target Cancer Cells on Repeated Exposure

The inventors investigated the capacity of SLC7A5 (LAT1) or SLC7A11(xCT)-modified CAR-T cells to kill target cancer cells on repeated exposure. CAR-T cells and AML cells (THP1) were incubated at a ratio of 1:5 respectively and fresh AML cells were added at 0 hours, 48 hours, 96 hours, 128 hours. The viability of the AML cells was assessed by flow cytometry Time 1(48 hours), Time 2 (96 hours), time 3 (128 hours), time 4 (148 hours).

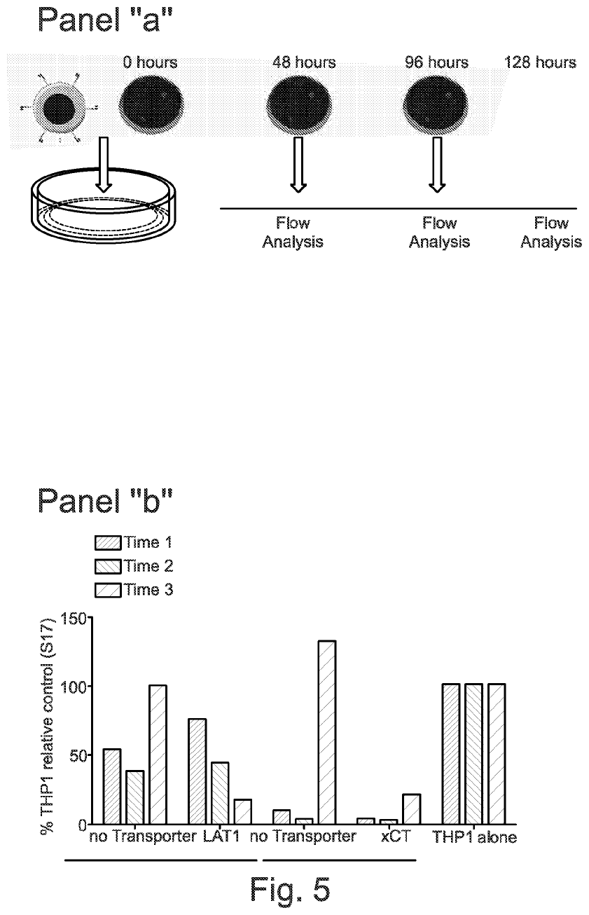

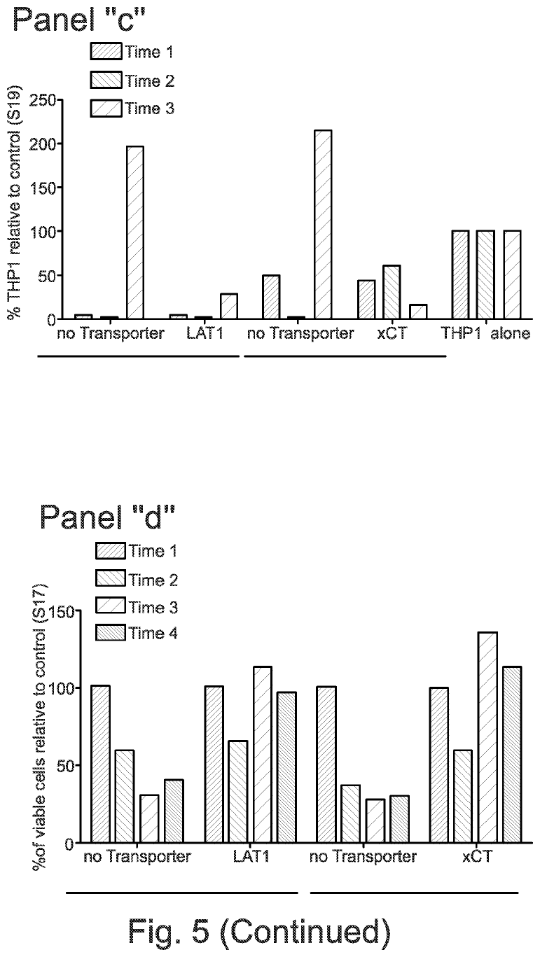

The data show that the unmodified ‘no transporter’ CAR-T cells lost the ability to kill after repeated exposure. However, killing capacity was preserved in LAT1 or xCT modified CAR-T cells. Data for 3 individual donors is shown in FIG. 5, panels b, c and d.

### 6.1 Modified CAR-T Cells Demonstrate Increased Tumour Cell Clearance and Prolonged Murine Survival In Vivo

The inventors investigated the ability of SLC7A5 (LAT1) or SLC7A11(xCT)-modified CAR-T cells to clear tumour cells and prolonged survival of mice treated with the modified CAR-T cells.

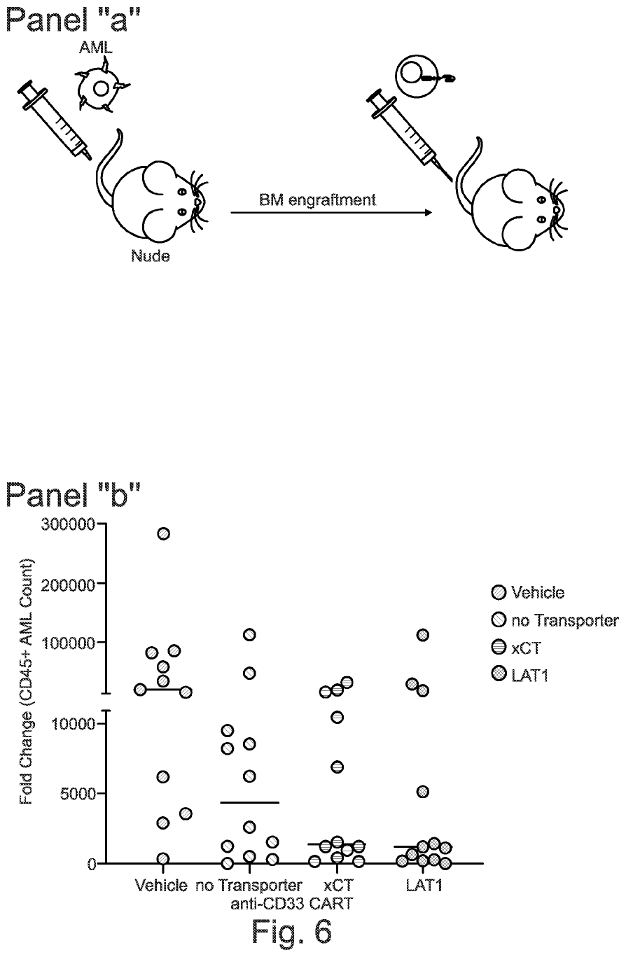

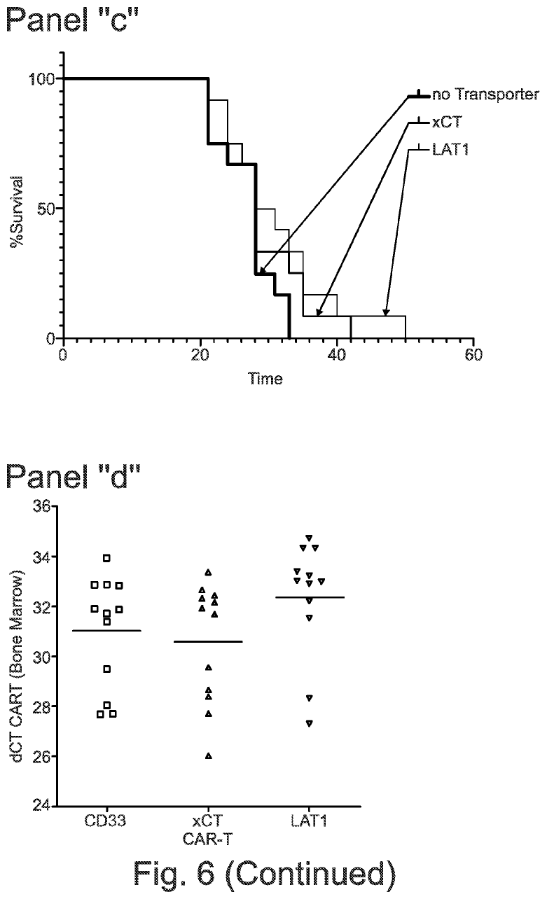

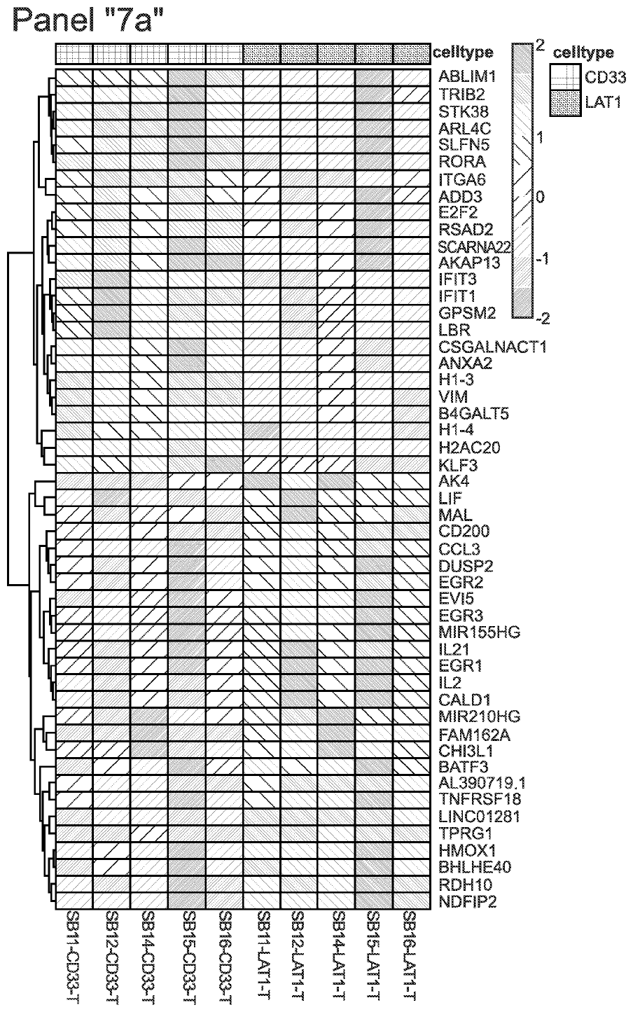

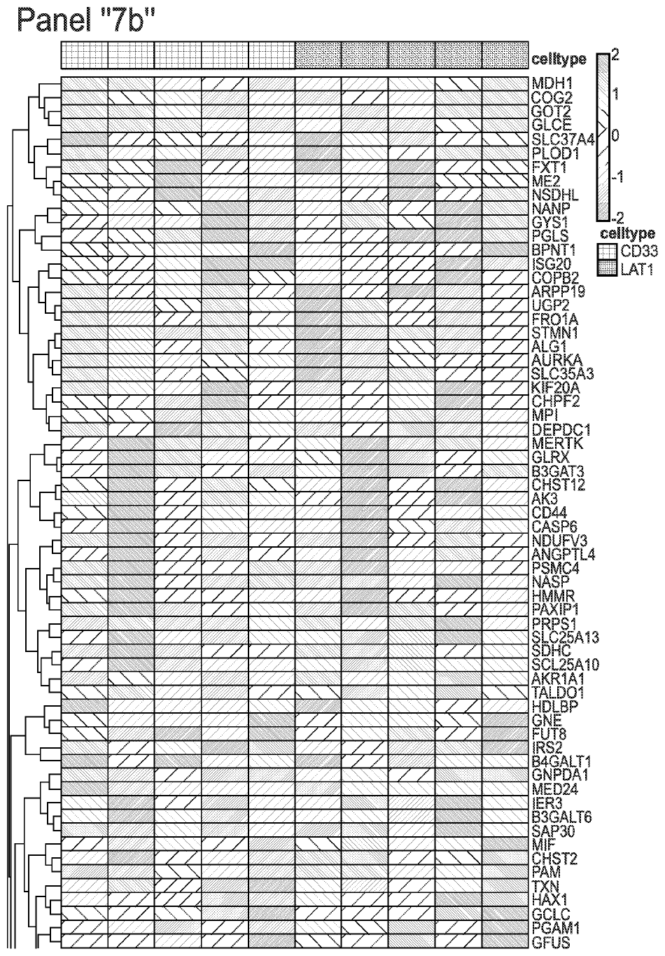

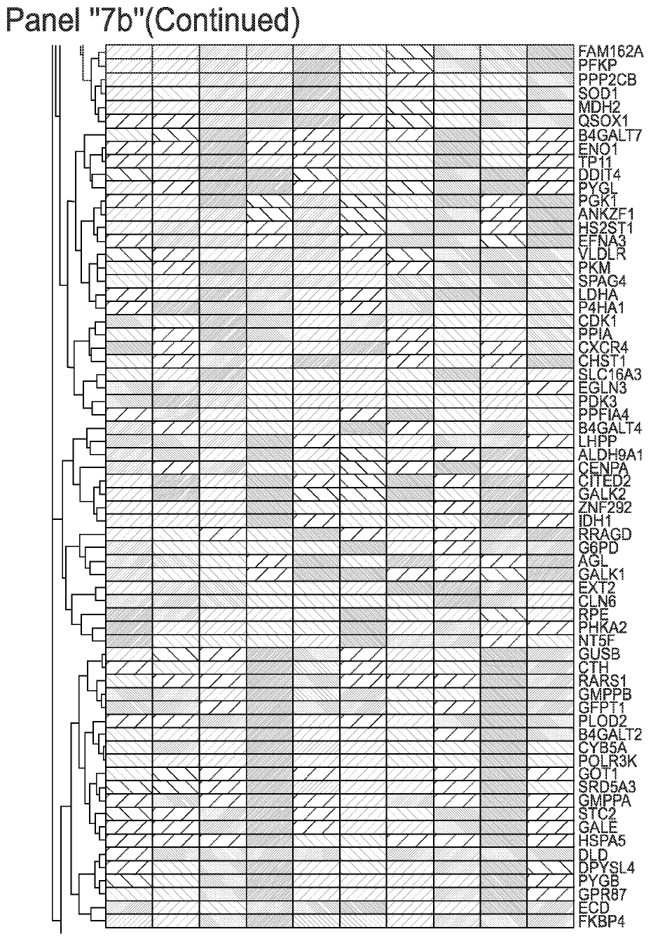

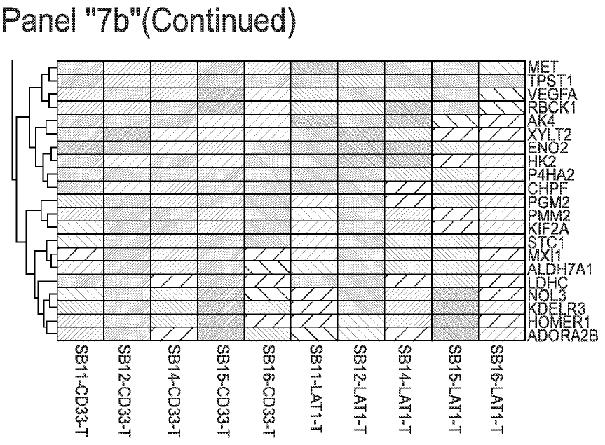

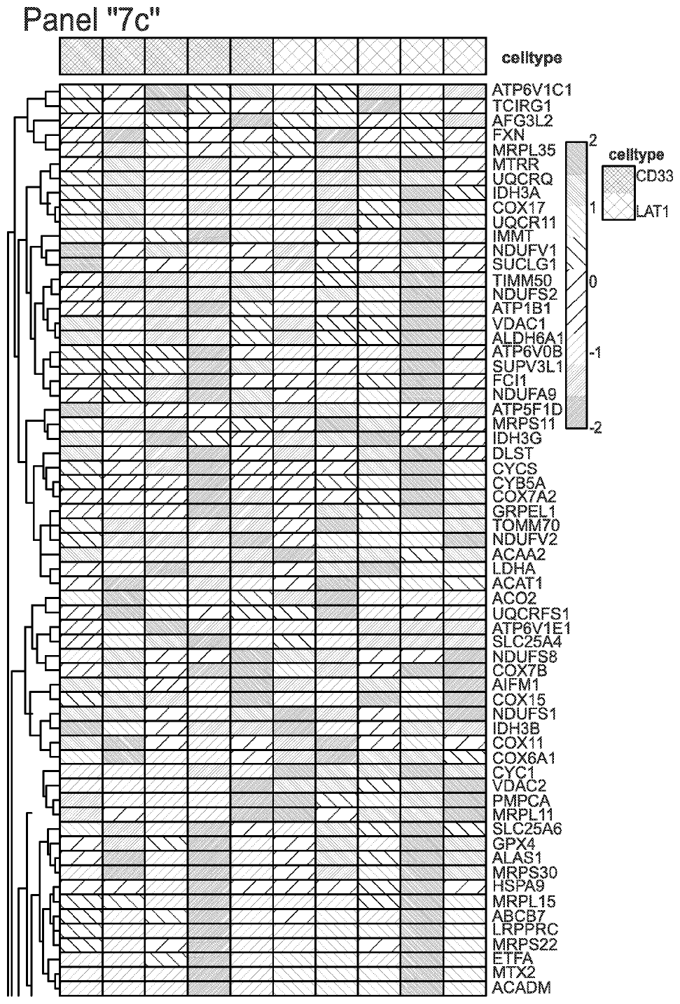

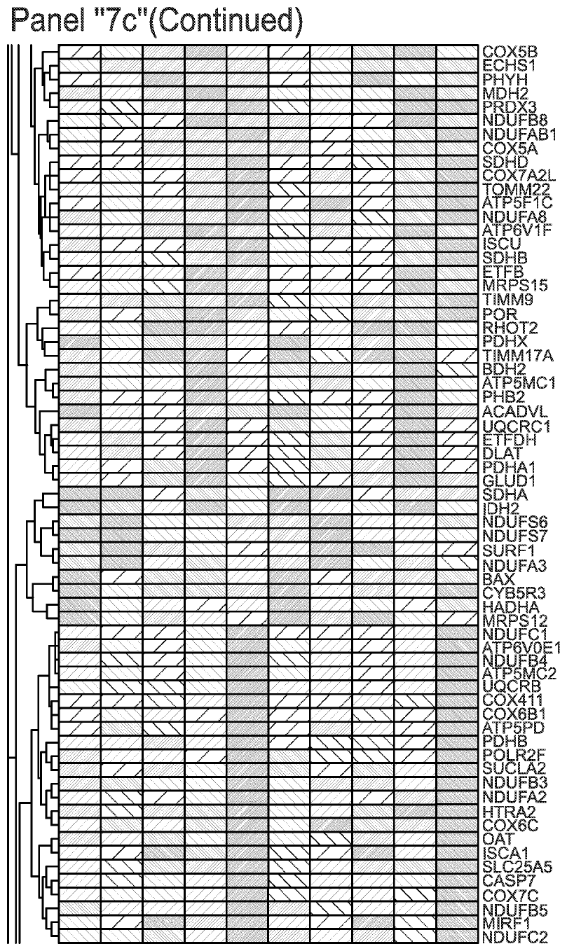

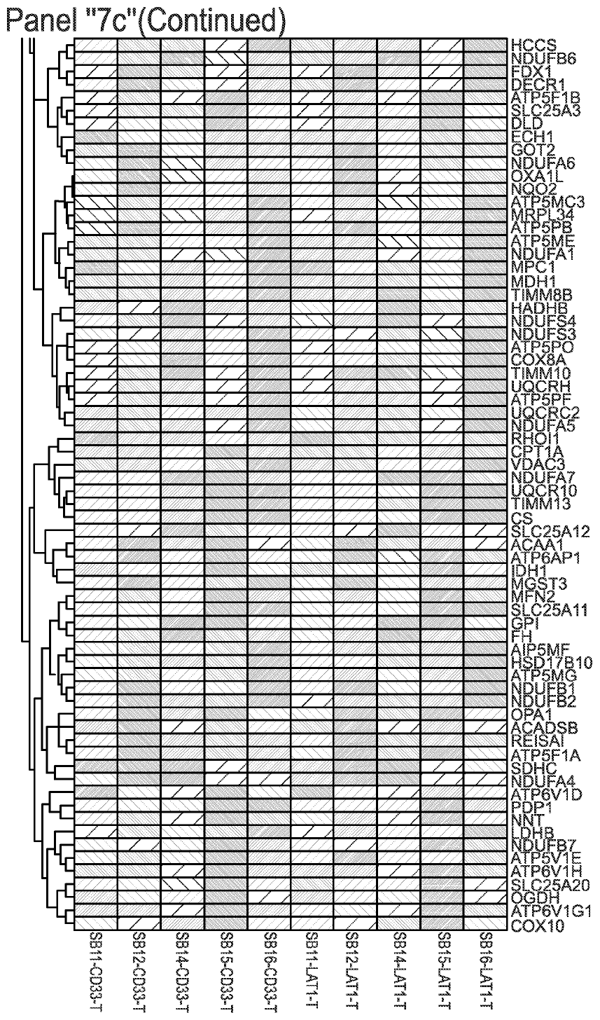

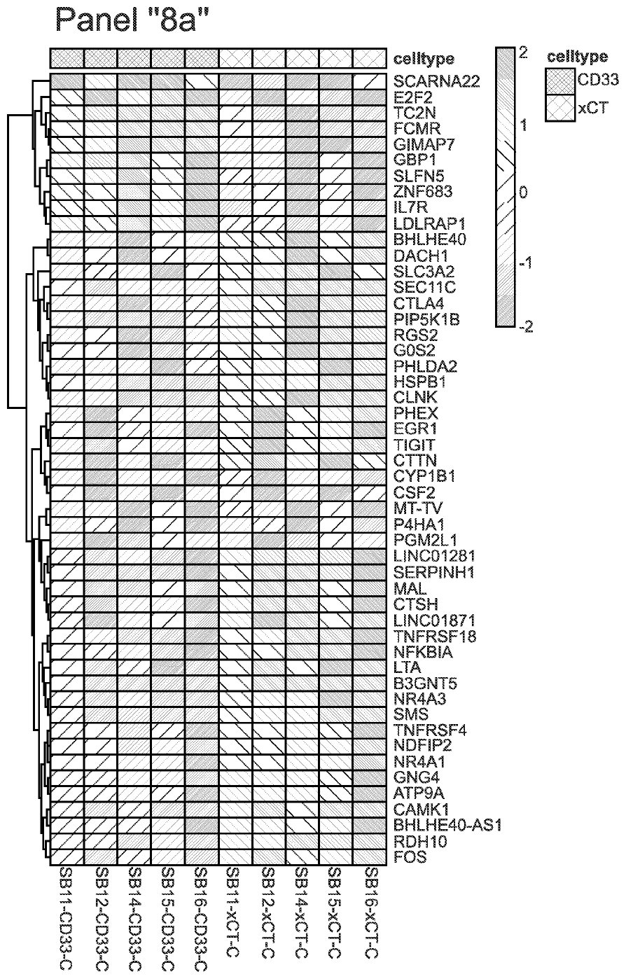

NOG-SCID mice were engrafted with 1×10⁶ CD33+tumour cells (HL60). Once bone marrow engraftment was confirmed on femoral biopsy, 2.5×10⁶ CAR-T cells were administered by tail vein injection. The data shown in FIG. 6 panel “b” shows a LAT-1 or xCT modified CAR-T cells have enhanced clearance of tumour cells from the bone marrows of the mice compared to standard ‘no transporter’ CAR-T cells.

Mice treated with the LAT-1 or xCT modified CAR-T cells also have prolonged survival compared to mice treated with standard ‘no transporter’ CAR-T cells. This data is shown in FIG. 6 panel “c”.

The inventors also demonstrated that the modified CAR-T cells are detectable within the bone marrow environment of treated mice, as measured by qRT-PCR, this data is shown in FIG. 6 panel “d”.

### 7.1 Modified CAR-T Cells Have a Series of Unique, Downstream Transcriptomic Adaptations to the Tumour Microenvironment Compared to Unmodified CAR-T Cells

The inventors have demonstrated that both SLC7A5 (LAT1) and SLC7A11 (xCT) modified CAR-T cells have a series of unique, downstream transcriptomic adaptations to the tumour microenvironment compared to unmodified CAR-T cells.

### SLC7A5 (LAT1) Modified CAR-T Cells:

CAR-T cells were transduced, and expanded before starvation of Tryptophan for a 5 hour period. CAR-T cells were then cultured with THP1 tumour cells (1:1) for 48 hours, in media containing 100 uM Tryptophan in the presence of THP1 leukaemia cells (1:1) for 48 hrs. CAR-T cells were then sorted by flow cytometry and subjected to RNA sequencing.

SLC7A11 (xCT) Modified CAR-T Cells:

CAR-T cells were transduced, and expanded before starvation of Cystine for a 5 hour period. CAR-T cells were then cultured with THP1 tumour cells (1:1) for 48hours, in media containing 100uM Cystine in the presence of THP1 leukaemia cells (1:1) for 48 hrs. CAR-T cells were then sorted by flow cytometry and subjected to RNA sequencing.

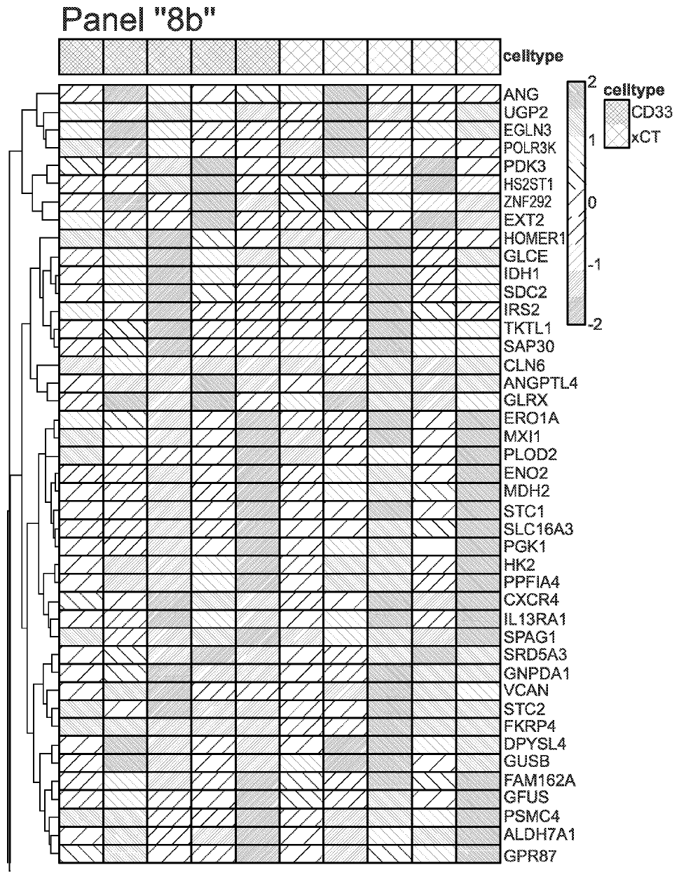

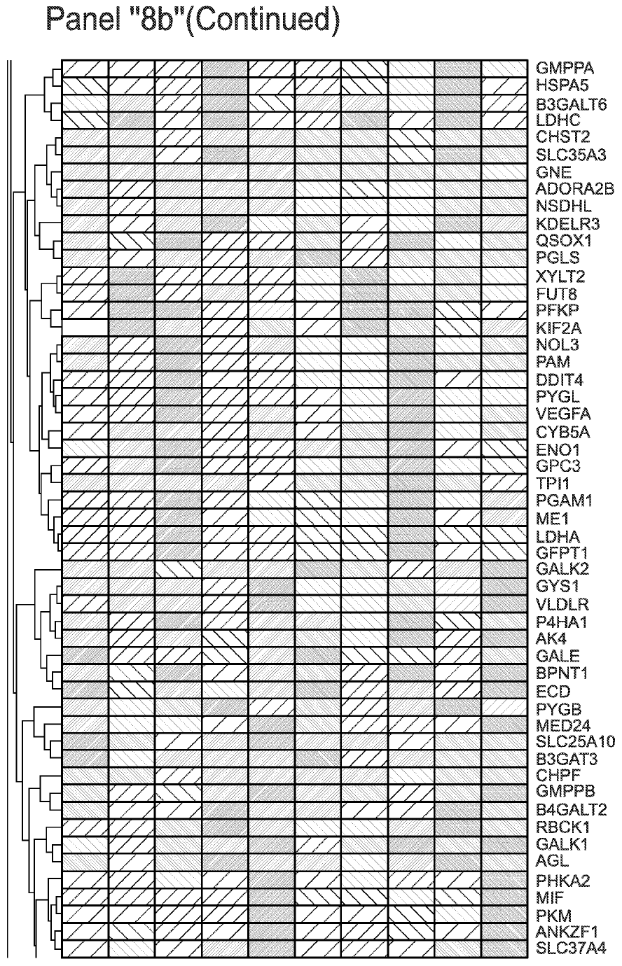

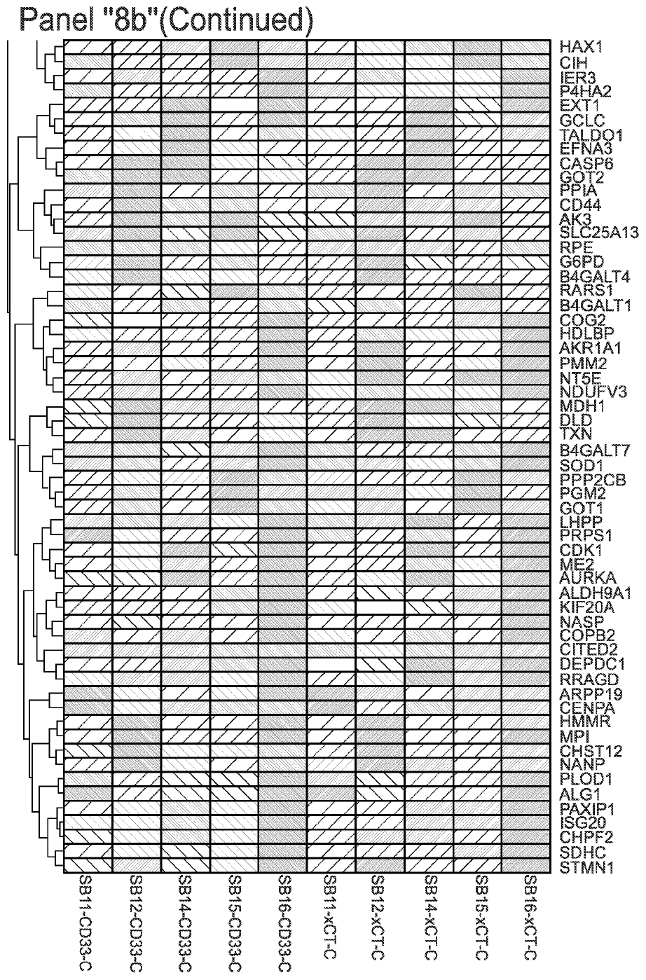

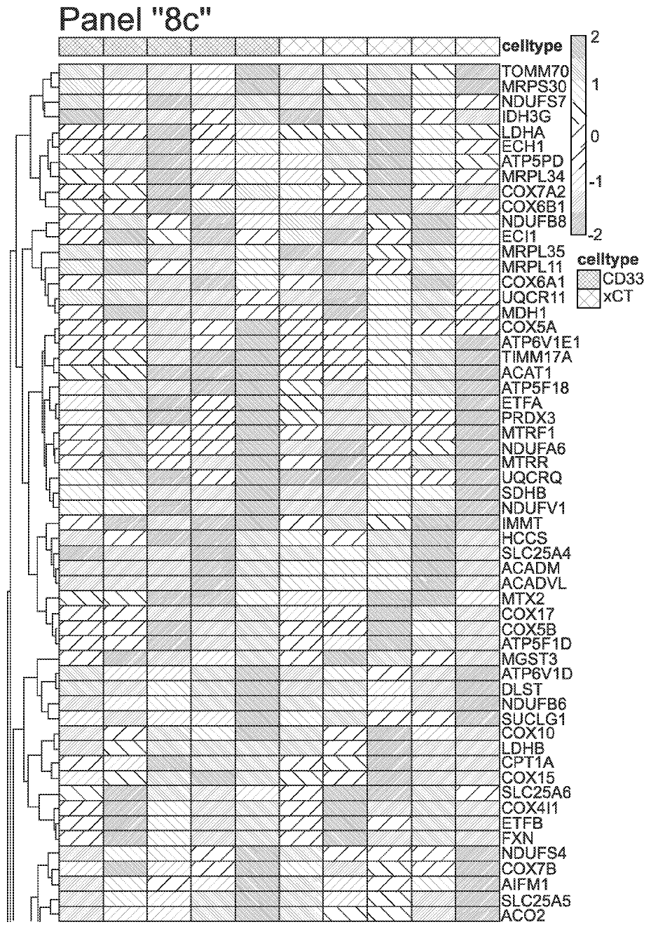

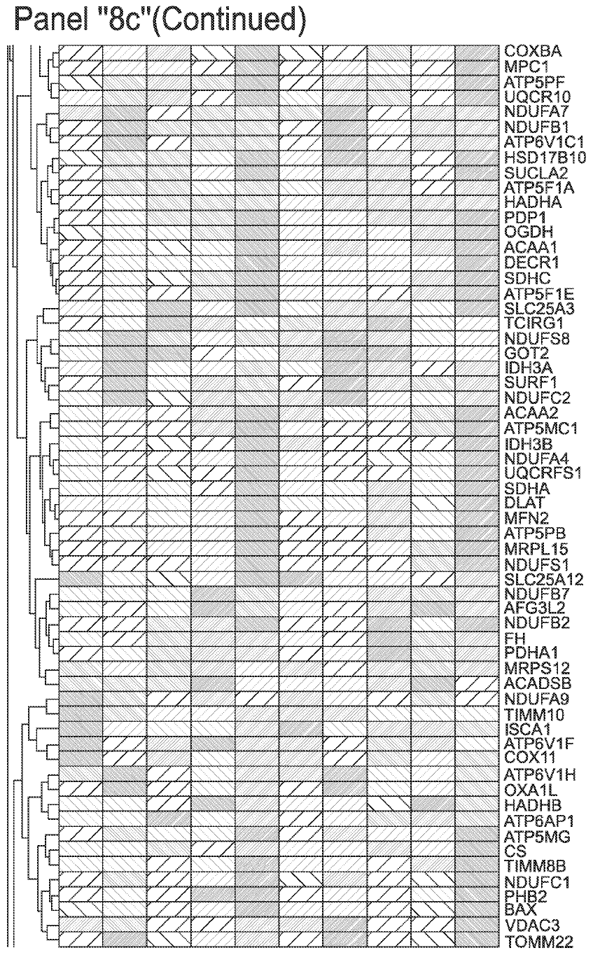

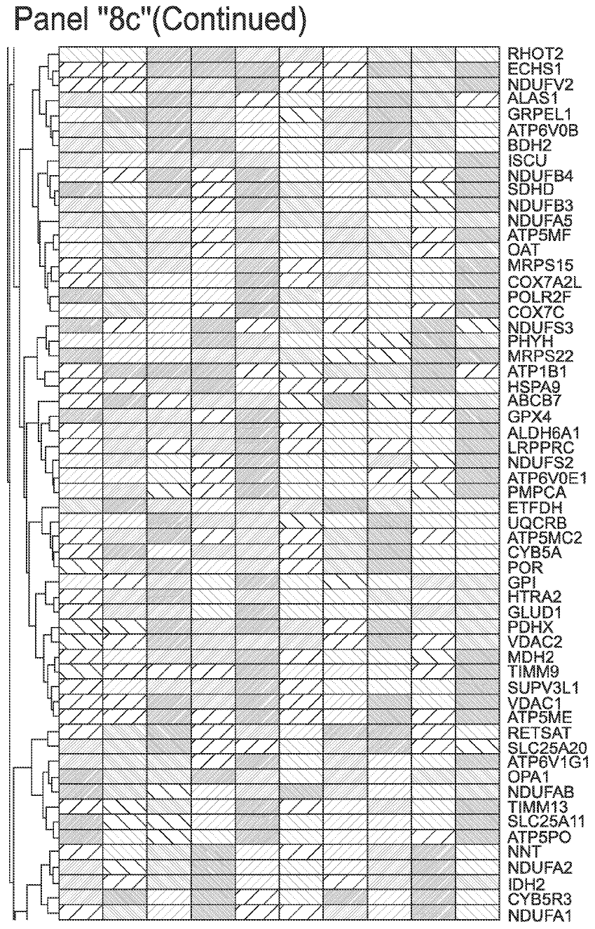

The data in FIG. 7 and FIG. 8 are represented as heatmaps of differential expression analysis, showing genes that are significantly up-/down-regulated in the modified CAR-T cells.

FIG. 7 panel “a” shows the top 50 genes that are significantly up-/down-regulated in the modified CAR-T cells. Panels 7 “b” and “c” show the top genes that are significantly up-/down-regulated in the glycolysis (7″b″) and oxidative phosphorylation (7“c”) pathways in CD33 vs CD33-LAT1 CAR-T cells. The genes shown on the vertical axis of the heatmaps in panels “b” and “c” are listed in Table 2 in the order in which they appear on their respective heatmap.

FIG. 8 panel “a” shows the top 50 genes that are significantly up-/down-regulated in the modified CAR-T cells. Panels 8 “b” and “c” show the top genes that are significantly up-/down-regulated in the glycolysis (7“b”) and oxidative phosphorylation (7“c”) pathways in CD33 vs CD33-xCT CAR-T cells. The genes shown on the vertical axis of the heatmaps in panels “b” and “c” are listed in Table 2 in the order in which they appear on their respective heatmap.

### 8.1 Modified CAR-T are Metabolically Adapted to Standard and Low Amino Acid Conditions.

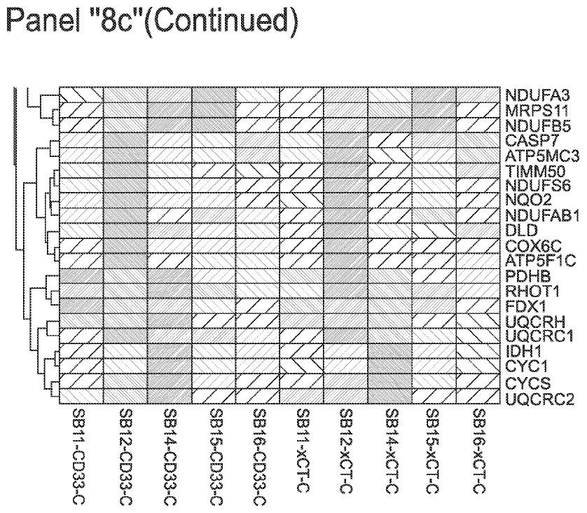

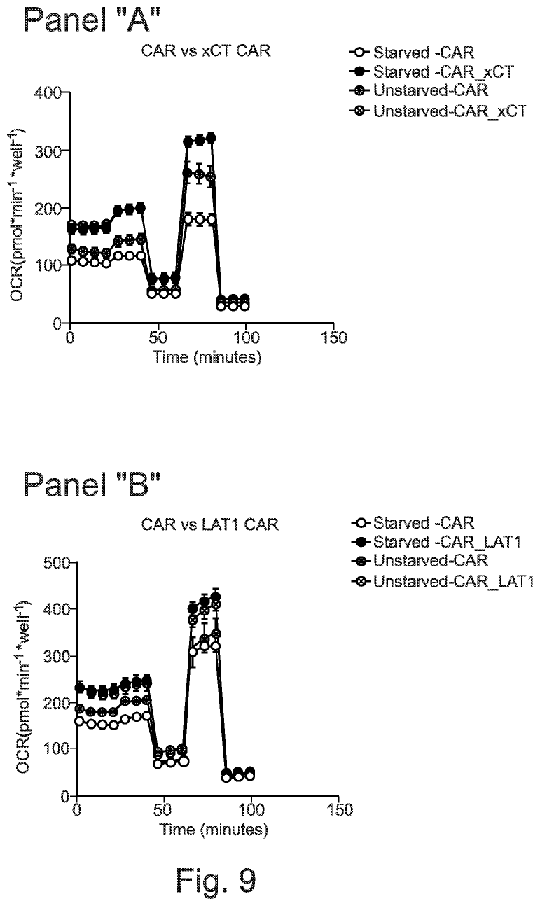

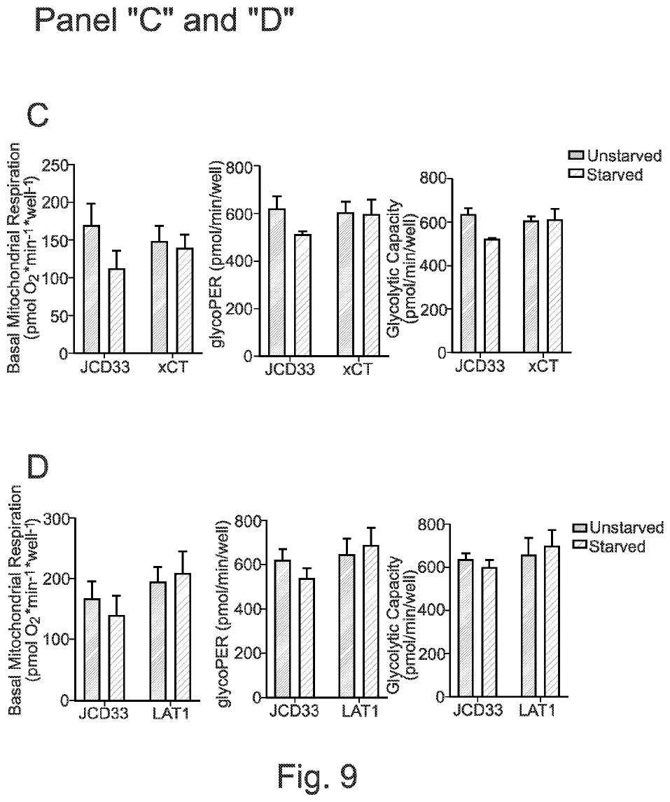

The inventors demonstrated that the SLC7A5 (LAT1) or SLC7A11(xCT)-modified CAR-T are metabolically adapted to standard and low amino acid conditions. This was demonstrated using representative seahorse assay for unmodified and modified CAR-T cells. The data in FIG. 9 panel A shows SLC7A11(xCT) modified CARs compared to unmodified CARs (JCD33). FIG. 9 panel B shows SLC7A5(LAT1) modified CARs compared to unmodified CARs (JCD33). Panels C and D are pooled analysis showing unmodified CAR-T cells have lower basal, ATP linked and maximal respiratory response capacity compared to transporter modified CAR-T cells (panel C: CAR vs xCT CAR and panel D: CAR vs LAT1 CAR).

### 9 Summary

The results reported in these studies demonstrate that cells of the invention (comprising nucleic acid constructs encoding a CAR and either SLC7A5 or SLC7A11) can be manufactured and purified in the manner that is required in order to generate therapeutically effective numbers of cells. Furthermore, the cells of the invention exhibit increased expression of amino acid transporters encoded by the nucleic acid constructs with which they have been transduced (in the studies set out above, SLC7A5 or SLC7A11), and increased uptake of amino acids in vitro.

Beyond this increase in expression of amino acid transporters, the cells of the invention also exhibit biological properties that make them suitable for therapeutic use, particularly in conditions of reduced amino acid concentrations that replicate those observed in cancer. These conditions, which have immunosuppressive effects, are believed to contribute to many failings observed in respect of immunotherapies.

As demonstrated in the results, the cells of the invention exhibit an ability to kill cancer cells with a degree of effectiveness comparable to that of control cells expressing CARs. The results also demonstrate that the cells of the invention exhibit an ability to kill cancer cells on repeat exposure over time, this is in contrast to the control cells expressing CARs, which do not retain the ability to kill cancer cells on repeat exposure over time. This indicates their therapeutic potential.

As demonstrated by the results, the cells of the invention exhibit an increased ability to clear tumour cells. The data shows that SLC7A5 (LAT1) or SLC7A11(xCT)-modified CAR-T cells were able to clear tumour cells more effectively as compared to control cells expressing CARs. In addition, this lead to prolonged survival of mice treated with the cells of the invention. This shows that the cells of the invention demonstrate improved therapeutic potential as compared to control cells expressing CARs.

As demonstrated by the results, surprisingly, the cells of the invention exhibit unique transcriptomic adaptations to the tumour microenvironment evidenced by the upregulation and downregulation of specific genes profiles compared to control CAR T-cells in the same tumour conditions. These adaptations include in the glycolysis and oxidative phosphorylation pathways advantageous changes in glycolysis and oxidative phosphorylation. Consistent with this, evaluation of mitochondrial metabolism confirms that modified CAR-T cells (cells of the invention) have a higher basal, ATP-linked and maximal respiratory response capacity under tumour conditions. This shows that the cells of the invention have improved transcriptomic and metabolic potential as compared to control cells expressing CARs.

Perhaps most importantly, the cells of the invention also demonstrate advantageous properties as compared to control cells expressing CARs. In particular, the cells of the invention are stimulated to proliferate to a much greater extent than control cells, when exposed to cells expressing the target molecule recognised by the fusion target-binding protein (in this case, CD33 expressed by cells of the cancer cell line THP-1). It will be appreciated that this improved response (leading, in the case of cells of the invention expressing SLC7A11, to an approximately 5-fold increase in cell numbers) will lead to the development of larger numbers of therapeutically effective cells in clinical use. Thus, the cells of the invention are likely to show improved therapeutic effectiveness, as compared to control CAR T cells, in use.

### 10 Exemplary Protocols Used in the Studies Described in the Examples

The following protocols were used in the studies set out in the Examples section. Accordingly, the conditions and reagents set out herein represent suitable embodiments for use in accordance with the invention. That said, the skilled person will understand that other, alternative, protocols may be capable of achieving the same results, and may also be used in the same manner.

### Protocols for the Production of Cells of the Invention

Retroviral Transduction of Human T cells

The following provides a protocol for the production of cells of the invention by transfection with nucleic acids of the invention.

### Day −2: Thaw Phoenix Ampho Cells

Late afternoon get Phoenix Ampho cells (retroviral packaging cell line for transduction of human cells) out of −80 and place in culture. Phoenix Ampho cells are grown in DMEM with 10% FCS, 1% L-glut (no antibiotics). Phoenix Ampho cells should never reach confluency. Typically put 2-3×106 Phoenix Ampho cells in eachT150 flask in 30 ml of media. On Monday you should have around 30-40×106 Phoenix Ampho cells.

### Day 1: Set up Phoenix Ampho Cells

Trypsinise Phoenix Ampho cells using TryLE and set up Phoenix Ampho cells at 8×106 cells/flask in 30 ml DM EM with 10% FCS and 1% L-glutamine (no antibiotics) (volume for T150 flask, scale as appropriate). Incubate cells overnight (37° C./5% CO2).

### Day 2: Transfection of Phoenix Ampho Cells

Phoenix Ampho cells should be 50-80% confluent on the day of transfection. The cells should then be transfected by the following method (for a T150 flask, scale as appropriate if using different flasks).

1. For each T150 flask of phoenix cells, place 12 μg of plasmid DNA (i.e. CAR plasmid)+12 μg pCI ampho plasmid into a 15 ml falcon and make up to 1800 μl with OptiMEM (Gibco) mixing gently with a pipette. To another 15 ml falcon add 1680 μl OptiMEM and add 120 μl Fugene 6 transfection reagent (available from stores) ensuring Fugene goes directly into OptiMEM rather than sticking to sides of tube; mix gently with a pipette. Then add the 1800 μl of OptiMEM/fugene mix to the tubes containing the plasmid DNA and mix gently with a pipette. Incubate at room temp for 45 mins. This allows fugene to form complexes with the DNA that have a neutral charge allowing DNA to be transported across the negatively charged Phoenix Ampho cell membrane.

2. Very gently replace the media on the Phoenix Ampno cells to 9 mls fresh DM EM with 10% FCS and glutamine then immediately overlay the DNA/fugene complexes or OptiMEM (for mock controls) onto the cells. Gently mix by north-south and east-west movements of the plate.

3. Incubate cells for 24 hours (37° C./5% CO2).

### Day 2: Activation of T Cells

T cells will not expand in the first 48 hours after activation, so typically activate as many T cells as you need (or more in case of cell death) for your transduction.

### Method Using Anti-CD3/CD28 Antibodies:

1. Lymphoprep a fresh leukocyte cone.

2. Count cells and culture at 1×106/ml in T cell media (1% human serum, 10% FCS, P/S, L-glut RPMI). Typically 200 mls per T150 flask.

3. Add IL-2 at 300 U/ml, add OKT3 (anti-CD3) at 30 ng/ml, add anti-CD28 mAB at 30 ng/ml (#MAB342-SP, R&D).

4. Incubate at 37° C./5% CO2 for 48 hours.

### Method Using Anti-CD3/CD28 Dynabeads:

1. Lymphoprep a fresh cone. Count cells and assume that 50% of PBMCs are CD3+ T cells.

2. Resuspend cells in a 15 ml falcon at 10×106 CD3+ T cells per ml of 5% human serum, PBS.

3. Add two Dynabeads® Human T-Activator CD3/CD28 per CD3+ cell. Washing dynabeads: vortex vial of beads for 30s. Remove required volume of beads and place in a 15 ml falcon. Add 1 ml of sterile PBS and mix well with a pipette. Place falcon on the dynabead magnet—dynabeads will stick to the edge of the falcon. Carefully remove the supernatant without disturbing the beads. Take falcon off the magnet and repeat wash step. Add dynabeads to your T cells in a small volume of PBS.

4. Incubate T cells on a tumbler at room temperature for at least an hour. T cells will bind dynabeads during this step, allowing selection of CD3+ T cells and activation at the same time.

5. Place cells on the dynabead magnet to remove non-bound cells. Count cells and culture at 1×106/ml in T cell media (1% human serum, 10% FCS, P/S, L-glut RPMI) with IL-2 300U/ml

6. Incubate at 37° C./5% CO2 for 48 hours.

### Day 3: Change Phoenix Ampho Media

Phoenix ampho cells will now be producing retrovirus containing your plasmid DNA, so take this into account when handling cells/supernatants. Place an autoclave bag inside your TC hood and place any plastics contaminated with retrovirus (cells/sups) within it. When you are 54 finished, seal the autoclave bag and place into an autoclave tin. Put any liquid waste in a waste pot and seal. Take retrovirus contaminated waste to wash-up ASAP.

Gently replace medium on Phoenix Ampho cells with fresh 21 mls/flask (volume for T150 flask, scale as appropriate) of DMEM +10% FCS+2mM L-glutamine (no antibiotics). Incubate the cells for a further 24 hours.

### Day 4: Transduction of Human T-Cells

1. Add 2 mls of retronectin (30 μg/ml) (#T100B—Takara RetroNectin® Recombinant Human Fibronectin Fragment) to each of the required number of wells of a 6-well plate (non tissue culture-treated) and incubate at room temperature for 3 hours (can also be set up the day before and coated overnight in the fridge). Remember to include wells for mock-transfected controls in the experiment. Culture plates are coated in retronectin to co-localise T cells and virus particles to allow efficient transduction of cells

2. Remove retronectin (it can be re-used until it has run out) and block wells with 2.5 ml of sterile PBS/2% BSA/well for 30mins. Remove the blocking solution and wash wells twice with 2.5 mls of sterile PBS (keep last PBS wash on well until ready to add virus).

3. Pre-warm some T cell media.

4. Pre-warm centrifuge for spinfection by spinning with empty buckets at 3160 rpm/2000 g for 60 mins@32° C. This can be interrupted when ready to do spinfection.

5. Harvest retrovirus-containing culture supernatant from Phoenix Ampho cells and spin down (1500 rpm for 5 mins). Transfer retrovirus sup to fresh tubes. Some people filter their retrovirus using a 0.45 μm filter, to remove contaminating Phoenix ampho cells, but this could decrease retroviral titre. If necessary, the virus can be snap frozen on dry ice/ethanol slurry and stored at −80° C., but titre is halved with every freeze thawing.

6. Spinfection: Add 2 ml/well of virus supernatant (or mock supernatant) to retronectin coated wells and spin at 3160 rpm/2000 g for 2 hours@32° C.

7. 45 mins before this spin finishes, prepare the T cells due to be transduced. Harvest T cells and count. Resuspend T cells at 1×106 in T cell media+IL2 (100 U/ml) and incubate (37° C./5% CO2) for 15-20 mins to allow cells to recover from centrifugation.

8. When virus has finished spinning, remove supernatants and wash wells once with PBS (2.5 ml/well).

9. Remove PBS from virus/retronectin coated plate and add T cells due to be transduced (2 ml/well). Ensure cells evenly distributed over plate by rocking north:south and east:west. Spin plates at 1300 rpm for 5 mins.

10. Place plates in incubator (37° C./5% CO2).

### Day 5: Feed Transduced T Cells

Add another 6 ml of T cell media+IL2 (100 IU/ml) to each well of T cells and return to incubator (37° C./5% CO2).

### Determining CAR Transduction Efficiency

The efficiency of methods for transducing cells to produce cells of the invention may be determined using the following procedure. CAR T cell transduction efficiency is determined 4 days post-spinfection. Take samples from mock and CAR T cell wells and stain as follows:
1. Wash ×1 with FACs buffer (10% FCS, PBS)
2. Stain with CD34-APC (1 μl/sample), CD4-FITC (2 μl/sample) and CD8-PE (1 μl/sample) in 50 μl of FACs buffer
3. Incubate for 20 mins on ice
4. Wash ×1 with FACS buffer
5. Resuspend cells in 200 μl of FACS buffer and analyse by flow cytometry.

### Sorting Cells of the Invention (Such as CAR T Cells) by CD34 Magnetic-Activated Cell Sorting

CAR-transduced cells (such as T cells) are sorted as follows:
1. Spin down T-cells at 1500 rpm, 5 mins and pour off supernatant.
2. Resuspend cells in 10 ml cold MACS buffer and spin 1500 rpm, 5 mins and pour off supernatant.
3. Resuspend cells in 300 μl cold MACS buffer, add 100 μl FcR blocking agent and 100 μl CD34 microbeads (Miltenyi Biotech 130-046-702). These quantities are suitable for up to 108 cells—if more than that, scale up accordingly.
4. Mix well and incubate for 30 mins in the fridge (2-8° C.). 56
5. Wash in 50 ml cold MACS buffer and spin 1500 rpm, 5 mins and pour off supernatant.
6. Resuspend cells in 500 μl cold MACS buffer and load cell suspension onto an MS column that has been pre-rinsed with 500 μl cold MACS buffer.
7. Allow cells to drip through by gravity flow and wash columns 3 times with 500 μl cold MACS buffer.
8. Remove columns from the magnet and flush through with 1 ml cold MACS buffer, collecting the cells in a sterile tube.
9. Centrifuge sorted CAR T cells and resuspend in normal T cell media (1% human serum, 10% FBS, P/S, L-glut, 100 U/ml IL-2, RPMI 1640) at a concentration of 1×106 CAR T cells per ml.
10. Check purity of CAR T cells by CD34 surface antibody staining the following day.

### Immunoblotting of Sorted CAR-T Cells

1. Sorted CAR-T cells were washed in PBS and lysed with RIPA lysis buffer (20 nM Tris-HCl pH7.5, 150 nM NaCl, 2 mM EDTA, 1.0% Triton X-100) containing cOmplete™ EDTA-free protease inhibitors (Roche) and PhosSTOP™ phosphatase inhibitors (Sigma).
2. Protein amounts in cell lysates were quantified by Bradford assay.
3. Electrophoresis of equal amounts of protein per test condition was performed in separate wells of a 4-20% Tris-Glycine SDS-PAGE gel (BioRad) at 150V for 1 hour.
4. The separated proteins were transferred to PVDF membranes using the TransBlot Turbo system (BioRad).
5. Protein bands were detected using primary antibodies to SLC7A5, SLC7A11 and actin. HRP-conjugated secondary antibodies of either goat anti-rabbit (CST, 7074S) or horse anti-mouse (CST, 7076S) were used for primary antibody detection.
6. Blots were developed using ECL substrate (BioRad), exposed to CL-Xposure X-ray film (ThermoFisher Scientific) or documented using the ChemidocMP system (BioRad).

### Determination of CAR-T Cell Immunophenotype

1. To determine the expression of T-cell exhaustion markers or naive/effector/memory phenotype CAR-T cells were washed in ice cold PBS then stained with fluorophore-conjugated anti-PD-1, anti-TIM3, anti-LAG1, anti-TIGIT antibodies or anti-CD45RA, CCR7 and anti- CD62L (all BioLegend) on ice for 30 min. Propidium iodide was used to define viable versus dead cells.
2. Fluorescence data was acquired using a CytoFLEX cytometer (Beckman Coulter).

Normalised population statistics—including the median fluorescence intensities (MFI) were determined using FlowJo (BD Biosciences, formerly developed by FlowJo LLC).

### Proliferation and Cytotoxicity Assays

1. CAR-T cells were cultured in 96-well flat bottom plates with THP-1 target cells and 200 μL culture media supplemented with 0.1% 3-mercaptoethanol (Thermo Fisher Scientific) at 37° C., 5% CO2 for 7 days.
2. CAR-T cell proliferation and viability or AML viability was determined by flow cytometry using anti-CD34 antibody to gate on CAR-T cells and propidium iodide to discriminate dead cells.
3. Analysis was performed using a CytoFLEX Flow Cytometer (Beckman Coulter).
4. Distinct populations of proliferating cells are depicted as histograms generated from data analysis using the FlowJo Software.

### Re-Exposure Assay

1. 1×105 CAR T-cells were plated per well in combination with 5×105 THP1 target tumour cells per well in 96-well plate flat bottom plate
2. For wells containing unmodified (CD33) and modified (CD33-LAT1) CAR-T cells, the cells were cultured in media composed of RPMI-10% FBS+β-mercaptoethanol+200 uM recombinant IL-2
3. For wells containing unmodified (CD33) and modified (CD33-xCT) CAR-T cells, the cells were cultured in media composed of RPMI-10% FBS+200 uM recombinant IL-2
4. The percentage of viable cells was determined by propidium iodide staining using flow cytometry days 0, 3, 7, 9 and 12.
5. After each flow cytometry read 5×105 THP1 target tumour cells were re-added (except day 0)

### In Vivo Modelling of CAR-T Cells

NOG-SCID mice were engrafted with 1×106 CD33+ tumour cells (HL60).
Once bone marrow engraftment was confirmed on femoral biopsy by flow cytometry, 2.5×106 CAR-T cells were administered by tail vein injection.
Murine survival was tracked over time.
AML disease burden and CAR-T cell infiltration in the bone marrows of sacrificed mice was determined by flow cytometry staining and RT-qPCR.

### RNA-sequencing of CAR-T Cells

1. Pre-conditioning of unmodified (CD33) and modified (CD33-LAT1) CAR-T cells prior to RNA sequencing
   CAR-T cells were transduced, expanded in 100 um Tryptophan for 24 hrs, before starvation of Tryptophan for a 5 hour period. CAR-T cells were then cultured in media containing 100uM Tryptophan in the presence of THP1 leukaemia cells (1:1) for 48 hrs. CAR-T cells were then sorted by flow cytometry.
2. Pre-conditioning of unmodified (CD33) and modified (CD33-xCT) CAR-T cells prior to RNA sequencing
   CAR-T cells were transduced, expanded in 100 um Cystine for 24 his, before starvation of Cystine for a 5 hour period. CAR-T cells were then cultured in media containing 100 uM Cystine in the presence of THP1 leukaemia cells (1 :1) for 48 hrs. CAR-T cells were then sorted by flow cytometry.
3. RNA was derived from the sorted CAR-T populations and prepared with the Illumina TruSeq RNA Sample Preparation Kit. They were sequenced on the Illumina HiSeq2000 platform. Sequencing reads were aligned to GRCh37 human genome using STAR RNA-Seq aligner software. Reads mapping to transcripts were counted by the same software. Normalisation of read counts and differential expression analysis comparing unmodified and modified CAR-T cells was performed using DESeq2 R Bioconductor package.

### Jurkat Seahorse Assay

1. Anti-CD33 CAR-Jurkat, anti-CD33 LAT1 CAR-Jurkat and anti-CD33 xCT-CAR Jurkat were cultured in RPMI-10% FBS
2. 3 hours prior to Seahorse analysis the CAR-Jurkat were placed in their respective amino acid depleted media:
   a. Anti-CD33 CAR-Jurkat and anti-CD33-LAT1 CAR-Jurkat were incubated in tryptophan free media
   b. Anti-CD33 CAR-Jurkat and anti-CD33 xCT-Jurkat were incubated in cystine free media
3. Cells were then immobilised onto CellTak-coated (Corning) Seahorse KFe96 plates (Agilent) at 2×105 cells per well in growth medium and incubated for 10 minutes.
4. Extracellular acidification rate (SCAR) and oxygen consumption rate (OCR) were measured on a Xfe96 Extracellular Flux Analyzer (Agilent)
5. Measurements were obtained under basal conditions and upon Oligomycin (2 μM), BAM15 (3 μM), Rotenone and Antimycin A (both 2 μM) staggered injections during the period of 80 minutes.

TABLE 1

| Gene name | Transport mechanism | Predominant |
|---|---|---|
| (Protein name) | (Coupling ions) | substrate(s) |
| SLC1A1 (EAAT3/EAAC1) | System X⁻_{AG}/Na⁺, | L-Glu, D/L-Asp, |
| | H⁺, K⁺ | L-Cys |
| SLC1A2 (EAAT2/GLT1) | System X⁻_{AG}/Na⁺, | L-Glu, D/L-Asp |
| | H⁺, K⁺ | |
| SLC1A3 (EAAT1/GLAST) | System X⁻_{AG}/Na⁺, | L-Glu, D/L-Asp |
| | H⁺, K⁺ | |
| SLC1A4 (ASCT1) | System ASC/ | L-Ala, L-Ser |
| | Antiporter | |
| SLC1A5 (ASCT2) | System ASC/ | L-Asp, L-Cys, |
| | Antiporter | L-Gln |
| SLC1A6 (EAAT4) | System X⁻_{AG}/Na⁺, | L-Glu, D/L-Asp |
| | H⁺, K⁺ | |
| SLC1A7 (EAAT5) | System X⁻_{AG}/Na⁺, | L-Glu, D/L-Asp |
| | H⁺, K⁺ | |
| SLC3A1 (rBAT) | Heavy chain | — |
| SLC3A2 (CD98hc/4F2hc) | Heavy chain | — |
| SLC6A5 (GlyT2) | Na⁺/Cl⁻ | LGly |
| SLC6A7 (PROT) | Na⁺/Cl⁻ | L-Pro |
| SLC6A9 (GlyT1) | Na⁺/Cl⁻ | L-Gly |
| SLC6A14 (ATB^{0, +}) | Na⁺/Cl⁻ | NAAs and CAAs |
| SLC6A15 (B⁰AT2) | Na⁺ | BCAAs |
| SLC6A17 (B⁰AT3) | Na⁺/Cl⁻ | NAAs |
| SLC6A18 (B⁰AT3) | Na⁺/Cl⁻ | L-Gly |
| SLC6A19 (B⁰AT1) | Na⁺ | NAAs |
| SLC6A20 (SIT1/IMINO) | Na⁺/Cl⁻ | L-Pro, |
| | | L-Hyp |
| SLC7A1 (CAT-1) | System y⁺/Uniporter | CAAs (L-Arg) |
| SLC7A2 (CAT-2) | System y⁺/Uniporter | CAAS (L-Arg) |
| SLC7A3 (CAT-3) | System y⁺/Uniporter | CAAs |
| SLC7A5 (LAT1) | System L/Antiporter | LNAAs |
| SLC7A6 (y⁺LAT2) | System y⁺L | CAAS and LNAAS |
| SLC7A7 (Y⁺LAT1) | System y⁺L | Cationic and NAAs |
| SLC7A8 (LAT2) | System L/Antiporter | LNAAs |
| SLC7A9 (b^{0, +}AT) | System b^{0, +} | CAAs, L-cystine |
| | | and NAAs |
| SLC7A10 (Asc-1) | System Asc/ | Small NAAs |
| | Antiporter | |
| SLC7A11 (xCT) | Sytem x⁺₀/Antiporter | L-Glu, L-Cys |
| SLC7A12 (Asc-2) | System Asc/ | L-Gly, L-Ala |
| | Antiporter | |
| SLC7A13 (AGT-1) | Antipoter | L-Asp L-Glu, L- |
| | | cystine |
| SLC7A14 | System C/ | CAAs |
| | Uniporter | |
| SLC15A3 (PHT2) | H⁺ | L-His |
| SLC15A4 (PHT1) | H⁺ | L-His |
| SLC16A10 (MCT10/TAT1) | Facilitated | Aromatic amino |
| | transporter | acids (L-Phe, L-Tyr, |
| | | L-Trp and L-Dopa) |
| | | [117] |
| SLC17A6 (VGLUT2) | Cl⁻/Uniporter | L-Glu |
| SLC17A7 (VGLUT1) | Cl⁻/Uniporter | L-Glu |
| SLC17A8 (VGLUT3) | Cl⁻/Uniporter | L-Glu |
| SLC25A2 (ORC2) | H⁺/Antiporter | L-Orn |
| | | L-Cit, L-Arg, L-His |
| SL C25A12 | H⁺/Antiporter | L-Glu, D/L-Asp |
| (AGC-1/Aralar1) | | |
| SL C25A13 | H⁺/Antiporter | L-Asp, L-Glu |
| (AGC-2/Aralar2) | | |
| SL C25A15 (ORNT1/ | H⁺/Antiporter | L-Orn, L-Cit |
| ORC1) | | |
| SL C25A18 (GC-2) | H⁺/Coupled; OH⁻/ | L-Glu |
| | Antiporter | |
| SL C25A22 (GC-1) | H⁺/Coupled; OH⁻/ | L-Glu |
| | Antiporter | |
| SL C25A38 | | L-Gly |
| SL C32A1 (VIAAT) | H⁺/Antiporter | L-Gly, GABA |
| SL C36A1 (PAT1) | H⁺ | GABA |
| S LC36A2 (PAT2) | H⁺ | L-Pro, L-Gly |
| S LC36A4 (PAT4) | H⁺ | L-Pro, L-Trp |
| SL C38A1 (SNAT1) | System A/Na⁺ | L-Gln |
| SL C38A2 (SNAT2) | System A/Na⁺ | L-Gln |
| SL C38A3 (SNAT3) | System N; Na⁺/ | L-Gln |
| | Coupled; H⁺/ | |
| | Antiporter | |
| SL C25A12 | H⁺/Antiporter | L-Glu, D/L-Asp |
| (AGC-1/Aralar1) | | |
| SL C25A13 | H⁺/Antiporter | L-Asp, L-Glu |
| (AGC-2/Aralar2) | | |
| SL C25A15 (ORNT1/ | H⁺/Antiporter | L-Orn, L-Cit |
| ORC1) | | |
| SL C25A18 (GC-2) | H⁺/Coupled; OH⁻/ | L-Glu |
| | Antiporter | |
| SL C25A22 (GC-1) | H⁺/Coupled; OH⁻/ | L-Glu |
| | Antiporter | |
| SL C25A38 | | L-Gly |
| SL C32A1 (VIAAT) | H⁺/Antiporter | L-Gly, GABA |
| SL C36A1 (PAT1) | H⁺ | GABA |
| SL C36A2 (PAT2) | H⁺ | L-Pro, L-Gly |
| SL C36A4 (PAT4) | H⁺ | L-Pro, L-Trp |
| S LC38A1 (SNAT1) | System A/Na⁺ | L-Gln |
| SL C38A2 (SNAT2) | System A/Na⁺ | L-Gln |
| SL C38A3 (SNAT3) | System N; Na⁺/ | L-Gln |
| | Coupled; H⁺/ | |
| | Antiporter | |

TABLE 2
The genes set out in the table below correspond to the
genes detailed in the heatmaps in FIGS. 7b, 7c, 8b
and 8c. The genes are provided in the table in the
order they appear on the vertical axis of the heatmap.

| Glycolysis | Oxidative | Glycolysis | Oxidative |
|---|---|---|---|
| CD33 vs | Phosphorylation | CD33 vs | Phosphorylation |
| LAT1 | CD33 vs LAT1 | xCT | CD33 vs xCT |
| (FIG. 7b) | (FIG. 7c) | (FIG. 8b) | (FIG. 8c) |
| MDH1 | ATP6V1C1 | ANG | TOMM70 |
| COG2 | TCIRG1 | UGP2 | MRPS30 |
| GOT2 | AFG3L2 | EGLN3 | NDUFS7 |
| GLCE | FXN | POLR3K | IDH3G |
| SLC37A4 | MRPL35 | PDK3 | LDHA |
| PLOD1 | MTRR | HS2ST1 | ECH1 |
| EXT1 | UQCRQ | ZNF292 | APT5PD |
| ME2 | IDH3A | EXT2 | MRPL34 |
| NSDHL | COX17 | HOMER1 | COX7A2 |
| NANP | UQCR11 | GLCE | COX6B1 |
| GYS1 | IMTT | IDH1 | NDUFB8 |
| PGLS | NDUFV1 | SDC2 | ECI1 |
| BPNT1 | SUCLG1 | IRS2 | MRPL35 |
| ISG20 | TIMM50 | TKTL1 | MRPL11 |
| COPB2 | NDUFS2 | SAP30 | COX6A1 |
| ARPP19 | ATP1B1 | CLN6 | UQCR11 |
| UGP2 | VDAC1 | ANGPTL4 | MDH1 |
| ERO1A | ALDH6A1 | GLRX | COX5A |
| STMN1 | ATP6V0B | ERO1A | APT6V1E1 |
| ALG1 | SUPV3L1 | MXI1 | TIMM17A |
| AURKA | ECI1 | PLOD2 | ACAT1 |
| SLC35A3 | NDUFA9 | ENO2 | ATP5F1B |
| KIF20A | ATP5F1D | MDH2 | ETFA |
| CHPF2 | MRPS11 | STC1 | PRDX3 |
| MPI | IDH3G | SLC16A3 | MTRF1 |
| DEPDC1 | DLST | PGK1 | NDUFA6 |
| MERTK | CYCS | HK2 | MTRR |
| GLRX | CYB5A | PPFIA4 | UQCRQ |
| B3GAT3 | COX7A2 | CXCR4 | SDHB |
| CHST12 | GRPEL1 | IL13RA1 | NDUFV1 |
| AK3 | TOMM70 | SPAG4 | IMMT |
| CD44 | NDUFV2 | SRD5A3 | HCCS |
| CASP6 | ACAA2 | GNPDA1 | SLC25A4 |
| NDUFV3 | LDHA | VCAN | ACADM |
| ANGPTL4 | ACAT1 | STC2 | ACADVL |
| PSMC4 | ACO2 | FKBP4 | MTX2 |
| NASP | UQCRFS1 | DPYSL4 | COX17 |
| HMMR | ATP6V1E1 | GUSB | COX5B |
| PAXIP1 | SLC25A4 | FAM162A | ATP5F1D |
| PRPS1 | NDUFS8 | GFUS | MGST3 |
| SLC25A13 | COX7B | PSMC4 | ATP6V1D |
| SDHC | AIFM1 | ALDH7A1 | DLST |
| SLC25A10 | COX15 | GPR87 | NDUFB6 |
| AKR1A1 | NDUFS1 | GMPPA | SUCLG1 |
| TALDO1 | IDH3B | HSPA5 | COX10 |
| HDLBP | COX11 | B3GALT6 | LDHB |
| GNE | COX6A1 | LDHC | CPT1A |
| FUT8 | CYC1 | CHST2 | COX15 |
| IRS2 | VDAC2 | SLC35A3 | SLC25A6 |
| B4GALT1 | PMPCA | GNE | COX4I1 |
| GNPDA1 | MRPL11 | ADORA2B | ETFB |
| MED24 | SLC25A6 | NSDHL | FXN |
| IER3 | GPX4 | KDELR3 | NDUFS4 |
| B3GALT6 | ALAS1 | QSOX1 | COX7B |
| SAP30 | MRPS30 | PGLS | AIFM1 |
| MIF | HSPA9 | XYLT2 | SLC25A5 |
| CHST2 | MRPL15 | FUT8 | ACO2 |
| PAM | ABCB7 | PFKP | COX8A |
| TXN | LRPPRC | KIF2A | MPC1 |
| HAX1 | MRPS22 | NOL3 | APT5PF |
| GCLC | ETFA | PAM | UQCR10 |
| PGAM1 | MTX2 | DDIT4 | NDUFA7 |
| GFUS | ACADM | PYGL | NDUFB1 |
| FAM162A | COX5B | VEGFA | ATP6V1C1 |
| PFKP | ECHS1 | CYB5A | HSD17B10 |
| PPP2CB | PHYH | ENO1 | SUCLA2 |
| SOD1 | MDH2 | GPC3 | ATP5F1A |
| MDH2 | PRDX3 | TPI1 | HADHA |
| QSOX1 | NDUFB8 | PGAM1 | PDP1 |
| B4GALT7 | NDUFAB1 | ME1 | OGDH |
| ENO1 | COX5A | LDHA | ACAA1 |
| TPI1 | SDHD | GFPT1 | DECR1 |
| DDIT4 | COX7A2L | GALK2 | SDHC |
| PYGL | TOMM22 | GYS1 | ATP5F1E |
| PGK1 | ATP5F1C | VLDLR | SLC25A3 |
| ANKZF1 | NDUFA8 | PAHA1 | TCIRG1 |
| HS2ST1 | ATP6V1F | AK4 | NDUFS8 |
| EFNA3 | ISCU | GALE | GOT2 |
| VLDLR | SDHB | BPNT1 | IDH3A |
| PKM | ETFB | ECD | SURF1 |
| SPAG4 | MRPS15 | PYGB | NDUFC2 |
| LDHA | TIMM9 | MED24 | ACAA2 |
| P4HA1 | POR | SLC25A10 | ATP5MC1 |
| CDK1 | RHOT2 | B3GAT3 | IDH3B |
| PPIA | PDHX | CHPF | NDUFA4 |
| CXCR4 | TIMM17A | GMPPB | UQCRFS1 |
| CHST1 | BDH2 | B4GALT2 | SDHA |
| SLC16A3 | ATP5MC1 | RBCK1 | DLAT |
| EGLN3 | PHB2 | GALK1 | MFN2 |
| PDK3 | ACADVL | AGL | ATP5PB |
| PPFIA4 | UQCRC1 | PHKA2 | MRPL15 |
| B4GALT4 | ETFDH | MIF | NDUFS1 |
| LHPP | DLAT | PKM | SLC25A12 |
| ALDH9A1 | PDHA1 | ANKZF1 | NDUFB7 |
| CENPA | GLUD1 | SLC37A4 | AFG3L2 |
| CITED2 | SDHA | HAX1 | NDUFB2 |
| GALK2 | IDH2 | CTH | FH |
| ZNF292 | NDUFS6 | IER3 | PDHA1 |
| IDH1 | NDUFS7 | P4HA2 | MRPS12 |
| RRAGD | SURF1 | EXT1 | ACADSB |
| G6PD | NDUFA3 | GCLC | NDUFA9 |
| AGL | BAX | TALDO1 | TIMM10 |
| GALK1 | CYB5R3 | EFNA3 | ISCA1 |
| EXT2 | HADHA | CASP6 | ATP6V1F |
| CLN6 | MRPS12 | GOT2 | COX11 |
| RPE | NDUFC1 | PPIA | ATP6V1H |
| PHKA2 | ATP6V0E1 | CD44 | OXA1L |
| NT5E | NDUFB4 | AK3 | HADHB |
| GUSB | ATP5MC2 | SLC25A13 | ATP6AP1 |
| CTH | UQCRB | RPE | ATP5MG |
| RARS1 | COX4I1 | G6PD | CS |
| GMPPB | COX6B1 | B4GALT4 | TIMM8B |
| GFPT1 | ATP5PD | RARS1 | NDUFC1 |
| PLOD2 | PDHB | B4GALT1 | PHB2 |
| B4GALT2 | POLR2F | COG2 | BAX |
| CYB5A | SUCLA2 | HDLBP | VDAC3 |
| POLR3K | NDUFB3 | AKR1A1 | TOMM22 |
| GOT1 | NDUFA2 | PMM2 | RHOT2 |
| SRD5A3 | HTRA2 | NT5E | ECHS1 |
| GMPPA | COX6C | NDUFV3 | NDUFV2 |
| STC2 | OAT | MDH1 | ALAS1 |
| GALE | ISCA1 | DLD | GRPEL1 |
| HSPA5 | SLC25A5 | TXN | ATP6V0B |
| DLD | CASP7 | B4GALT7 | BDH2 |
| DPYSL4 | COX7C | SOD1 | ISCU |
| PYGB | NDUFB5 | PP2CB | NDUFB4 |
| GPR87 | MTRF1 | PGM2 | SDHD |
| ECD | NDUFC2 | GOT1 | NDUFB3 |
| FKBP4 | HCCS | LHPP | NDUFA5 |
| MET | NDUFB6 | PRPS1 | ATP5MF |
| TPST1 | FDX1 | CDK1 | OAT |
| VEGFA | DECR1 | ME2 | MRPS15 |
| RBCK1 | ATP5F1B | AURKA | COX7A2L |
| AK4 | SLC25A3 | ALDH9A1 | POLR2F |
| XYLT2 | DLD | KIF20A | COX7C |
| ENO2 | ECH1 | NASP | NDUFS3 |
| HK2 | GOT2 | COPB2 | PHYH |
| P4HA2 | NDUFA6 | CITED2 | MRPS22 |
| CHPF | OXA1L | DEPDC1 | ATP1B1 |
| PGM2 | NQO2 | RRAGD | HSPA9 |
| PMM2 | ATP5MC3 | ARPP19 | ABCB7 |
| KIF2A | MRPL34 | CENPA | GPX4 |
| STC1 | ATP5PB | HMMR | ALDH6A1 |
| MXI1 | ATP5ME | MPI | LRPPRC |
| ALDH7A1 | NDUFA1 | CHST12 | NDUFS2 |
| LDHC | MPC1 | NANP | ATP6V0E1 |
| NOL3 | MDH1 | PLOD1 | PMPCA |
| KDELR3 | TIMM8B | ALG1 | ETFDH |
| HOMER1 | HADHB | PAXIP1 | UQCRB |
| ADORA2B | NDUFS4 | ISG20 | ATP5MC2 |
| | NDUFS3 | CHPF2 | CYB5A |
| | ATP5PO | SDHC | POR |
| | COX8A | STMN1 | GPI |
| | TIMM10 | | HTRA2 |
| | UQCRH | | GLUD1 |
| | ATP5PF | | PDHX |
| | UQCRC2 | | VDAC2 |
| | NDUFA5 | | MDH2 |
| | RHOT1 | | TIMM9 |
| | CPT1A | | SUPV3L1 |
| | VDAC3 | | VDAC1 |
| | NDUFA7 | | ATP5ME |
| | UQCR10 | | RETSAT |
| | TIMM13 | | SLC25A20 |
| | CS | | ATP6V1G1 |
| | SLC25A12 | | OPA1 |
| | ACAA1 | | NDUFA8 |
| | ATP6AP1 | | TIMM13 |
| | IDH1 | | SLC25A11 |
| | MGST3 | | ATP5PO |
| | MFN2 | | NNT |
| | SLC25A11 | | NDUFA2 |
| | GPI | | IDH2 |
| | FH | | CYB5R3 |
| | ATP5MF | | NDUFA1 |
| | HSD17B10 | | NDUFA3 |
| | ATP5MG | | MRPS11 |
| | NDUFB1 | | NDUFB5 |
| | NDUFB2 | | CASP7 |
| | OPA1 | | ATP5MC3 |
| | ACADSB | | TIMM50 |
| | RETSAT | | NDUFS6 |
| | ATP5F1A | | NQO2 |
| | SDHC | | NDUFAB1 |
| | NDUFA4 | | DLD |
| | ATP6V1D | | COX6C |
| | PDP1 | | ATP5F1C |
| | NNT | | PDHB |
| | LDHB | | RHOT1 |
| | NDUFB7 | | FDX1 |
| | ATP5F1E | | UQCRH |
| | ATP6V1H | | UQCRC1 |
| | SLC25A20 | | IDH1 |
| | OGDH | | CYC1 |
| | ATP6V1G1 | | CYCS |
| | COX10 | | UQCRC2 |

SEQUENCE INFORMATION

CAT-1 (SLC7A1),
Protein: UnitProt: P30825,
>sp|P30825|CTR1_HUMAN High affinity cationic amino acid transporter 1 OS = Homo
sapiens OX = 9606 GN = SLC7A1 PE = 1 SV = 1
MGCKVLLNIGQQMLRRKVVDCSREETRLSRCLNTFDLVALGVGSTLGAGVYVLAGAVARE
NAGPAIVISFLIAALASVLAGLCYGEFGARVPKTGSAYLYSYVTVGELWAFITGWNLILS
YIIGTSSVARAWSATFDELIGRPIGEFSRTHMTLNAPGVLAENPDIFAVIIILILTGLLT
LGVKESAMVNKIFTCINVLVLGFIMVSGFVKGSVKNWQLTEEDFGNTSGRLCLNNDTKEG
KPGVGGFMPFGFSGVLSGAATCFYAFVGFDCIATTGEEVKNPQKAIPVGIVASLLICFIA
YFGVSAALTLMMPYFCLDNNSPLPDAFKHVGWEGAKYAVAVGSLCALSASLLGSMFPMPR
VIYAMAEDGLLFKFLANVNDRTKTPIIATLASGAVAAVMAFLFDLKDLVDLMSIGTLLAY
SLVAACVLVLRYQPEQPNLVYQMASTSDELDPADQNELASTNDSQLGFLPEAEMFSLKTI
LSPKNMEPSKISGLIVNISTSLIAVLIITFCIVTVLGREALTKGALWAVELLAGSALLCA
VVTGVIWRQPESKTKLSFKVPFLPVLPILSIFVNVYLMMQLDQGTWVRFAVWMLIGFIIY
FGYGLWHSEEASLDADQARTPDGNLDQCK
SEQ ID NO: 1
Consensus Coding Sequence: CCDS9333.1: RefSeq: NM_003045.5,
ATGGGGTGCAAAGTCCTGCTCAACATTGGGCAGCAGATGCTGCGGCGGAAGGTGGTGGACTGTAGCCG
GGAGGAGACGCGGCTGTCTCGCTGCCTGAACACTTTTGATCTGGTGGCCCTCGGGGTGGGCAGCACAC
TGGGTGCTGGTGTCTACGTCCTGGCTGGAGCTGTGGCCCGTGAGAATGCAGGCCCTGCCATTGTCATC
TCCTTCCTGATCGCTGCGCTGGCCTCAGTGCTGGCTGGCCTGTGCTATGGCGAGTTTGGTGCTCGGGT
CCCCAAGACGGGCTCAGCTTACCTCTACAGCTATGTCACCGTTGGAGAGCTCTGGGCCTTCATCACCG
GCTGGAACTTAATCCTCTCCTACATCATCGGTACTTCAAGCGTAGCGAGGGCCTGGAGCGCCACCTTC
GACGAGCTGATAGGCAGACCCATCGGGGAGTTCTCACGGACACACATGACTCTGAACGCCCCCGGCGT
GCTGGCTGAAAACCCCGACATATTCGCAGTGATCATAATTCTCATCTTGACAGGACTTTTAACTCTTG
GTGTGAAAGAGTCGGCCATGGTCAACAAAATATTCACTTGTATTAACGTCCTGGTCCTGGGCTTCATA
ATGGTGTCAGGATTTGTGAAAGGATCGGTTAAAAACTGGCAGCTCACGGAGGAGGATTTTGGGAACAC
ATCAGGCCGTCTCTGTTTGAACAATGACACAAAAGAAGGGAAGCCCGGTGTTGGTGGATTCATGCCCT
TCGGGTTCTCTGGTGTCCTGTCGGGGGCAGCGACTTGCTTCTATGCCTTCGTGGGCTTTGACTGCATC
GCCACCACAGGTGAAGAGGTGAAGAACCCACAGAAGGCCATCCCCGTGGGGATCGTGGCGTCCCTCTT
GATCTGCTTCATCGCCTACTTTGGGGTGTCGGCTGCCCTCACGCTCATGATGCCCTACTTCTGCCTGG
ACAATAACAGCCCCCTGCCCGACGCCTTTAAGCACGTGGGCTGGGAAGGTGCCAAGTACGCAGTGGCC
GTGGGCTCCCTCTGCGCTCTTTCCGCCAGTCTTCTAGGTTCCATGTTTCCCATGCCTCGGGTTATCTA
TGCCATGGCTGAGGATGGACTGCTATTTAAATTCTTAGCCAACGTCAATGATAGGACCAAAACACCAA
TAATCGCCACATTAGCCTCGGGTGCCGTTGCTGCTGTGATGGCCTTCCTCTTTGACCTGAAGGACTTG
GTGGACCTCATGTCCATTGGCACTCTCCTGGCTTACTCGTTGGTGGCTGCCTGTGTGTTGGTCTTACG
GTACCAGCCAGAGCAGCCTAACCTGGTATACCAGATGGCCAGTACTTCCGACGAGTTAGATCCAGCAG
ACCAAAATGAATTGGCAAGCACCAATGATTCCCAGCTGGGCTTTTTACCAGAGGCAGAGATGTTCTCT
TTGAAAACCATACTCTCACCCAAAAACATGGAGCCTTCCAAAATCTCTGGGCTAATTGTGAACATTTC
AACCAGCCTCATAGCTGTTCTCATCATCACCTTCTGCATTGTGACCGTGCTTGGAAGGGAGGCTCTCA
CCAAAGGGGCGCTGTGGGCAGTCTTTCTGCTCGCAGGGTCTGCCCTCCTCTGTGCCGTGGTCACGGGC
GTCATCTGGAGGCAGCCCGAGAGCAAGACCAAGCTCTCATTTAAGGTTCCCTTCCTGCCAGTGCTCCC
CATCCTGAGCATCTTCGTGAACGTCTATCTCATGATGCAGCTGGACCAGGGCACCTGGGTCCGGTTTG
CTGTGTGGATGCTGATAGGCTTCATCATCTACTTTGGCTATGGCCTGTGGCACAGCGAGGAGGCGTCC
CTGGATGCCGACCAAGCAAGGACTCCTGACGGCAACTTGGACCAGTGCAAGTGA
SEQ ID NO: 2
LAT-1 (SLC7A5),
Protein: UnitProt: Q01650,
>sp|Q01650|LAT1_HUMAN Large neutral amino acids transporter small subunit 1
OS = Homo sapiens OX = 9606 GN = SLC7A5 PE = 1 SV = 2
MAGAGPKRRALAAPAAEEKEEAREKMLAAKSADGSAPAGEGEGVTLQRNITLLNGVAIIV
GTIIGSGIFVTPTGVLKEAGSPGLALVVWAACGVFSIVGALCYAELGTTISKSGGDYAYM
LEVYGSLPAFLKLWIELLIIRPSSQYIVALVFATYLLKPLFPTCPVPEEAAKLVACLCVL
LLTAVNCYSVKAATRVODAFAAAKLLALALIILLGFVQIGKGDVSNLDPNFSFEGTKLDV
GNIVLALYSGLFAYGGWNYLNFVTEEMINPYRNLPLAIIISLPIVTLVYVLTNLAYFTTL
STEQMLSSEAVAVDFGNYHLGVMSWIIPVFVGLSCFGSVNGSLFTSSRLFFVGSREGHLP
SILSMIHPQLLTPVPSLVFTCVMTLLYAFSKDIFSVINFFSFFNWLCVALAIIGMIWLRH
RKPELERPIKVNLALPVFFILACLFLIAVSFWKTPVECGIGFTIILSGLPVYFFGVWWKN
KPKWLLQGIFSTTVLCQKLMQVVPQET
SEQ ID NO: 3
Consensus Coding Sequence: CCDS10964.1: RefSeq: NM_003486.7,
ATGGCGGGTGCGGGCCCGAAGCGGCGCGCGCTAGCGGCGCCGGCGGCCGAGGAGAAGGAAGAGGCGCG
GGAGAAGATGCTGGCCGCCAAGAGCGCGGACGGCTCGGCGCCGGCAGGCGAGGGCGAGGGCGTGACCC
TGCAGCGGAACATCACGCTGCTCAACGGCGTGGCCATCATCGTGGGGACCATTATCGGCTCGGGCATC
TTCGTGACGCCCACGGGCGTGCTCAAGGAGGCAGGCTCGCCGGGGCTGGCGCTGGTGGTGTGGGCCGC
GTGCGGCGTCTTCTCCATCGTGGGCGCGCTCTGCTACGCGGAGCTCGGCACCACCATCTCCAAATCGG
GCGGCGACTACGCCTACATGCTGGAGGTCTACGGCTCGCTGCCCGCCTTCCTCAAGCTCTGGATCGAG
CTGCTCATCATCCGGCCTTCATCGCAGTACATCGTGGCCCTGGTCTTCGCCACCTACCTGCTCAAGCC
GCTCTTCCCCACCTGCCCGGTGCCCGAGGAGGCAGCCAAGCTCGTGGCCTGCCTCTGCGTGCTGCTGC
TCACGGCCGTGAACTGCTACAGCGTGAAGGCCGCCACCCGGGTCCAGGATGCCTTTGCCGCCGCCAAG
CTCCTGGCCCTGGCCCTGATCATCCTGCTGGGCTTCGTCCAGATCGGGAAGGGTGATGTGTCCAATCT
AGATCCCAACTTCTCATTTGAAGGCACCAAACTGGATGTGGGGAACATTGTGCTGGCATTATACAGCG
GCCTCTTTGCCTATGGAGGATGGAATTACTTGAATTTCGTCACAGAGGAAATGATCAACCCCTACAGA
AACCTGCCCCTGGCCATCATCATCTCCCTGCCCATCGTGACGCTGGTGTACGTGCTGACCAACCTGGC
CTACTTCACCACCCTGTCCACCGAGCAGATGCTGTCGTCCGAGGCCGTGGCCGTGGACTTCGGGAACT
ATCACCTGGGCGTCATGTCCTGGATCATCCCCGTCTTCGTGGGCCTGTCCTGCTTCGGCTCCGTCAAT
GGGTCCCTGTTCACATCCTCCAGGCTCTTCTTCGTGGGGTCCCGGGAAGGCCACCTGCCCTCCATCCT
CTCCATGATCCACCCACAGCTCCTCACCCCCGTGCCGTCCCTCGTGTTCACGTGTGTGATGACGCTGC
TCTACGCCTTCTCCAAGGACATCTTCTCCGTCATCAACTTCTTCAGCTTCTTCAACTGGCTCTGCGTG
GCCCTGGCCATCATCGGCATGATCTGGCTGCGCCACAGAAAGCCTGAGCTTGAGCGGCCCATCAAGGT
GAACCTGGCCCTGCCTGTGTTCTTCATCCTGGCCTGCCTCTTCCTGATCGCCGTCTCCTTCTGGAAGA
CACCCGTGGAGTGTGGCATCGGCTTCACCATCATCCTCAGCGGGCTGCCCGTCTACTTCTTCGGGGTC
TGGTGGAAAAACAAGCCCAAGTGGCTCCTCCAGGGCATCTTCTCCACGACCGTCCTGTGTCAGAAGCT
CATGCAGGTGGTCCCCCAGGAGACATAG
SEQ ID NO: 4
4F2hc (CD98 or SLC3A2, cell surface antigen heavy chain)
Protein: UnitProt: P08195,
>sp|P08195|4F2_HUMAN 4F2 cell-surface antigen heavy chain OS = Homo sapiens
OX = 9606 GN = SLC3A2 PE = 1 SV = 3
MELQPPEASIAVVSIPROLPGSHSEAGVOGLSAGDDSELGSHCVAQTGLELLASGDPLPS
ASQNAEMIETGSDCVTQAGLOLLASSDPPALASKNAEVTGTMSQDTEVDMKEVELNELEP
EKQPMNAASGAAMSLAGAEKNGLVKIKVAEDEAEAAAAAKFTGLSKEELLKVAGSPGWVR
TRWALLLLFWLGWLGMLAGAVVIIVRAPRCRELPAQKWWHTGALYRIGDLQAFQGHGAGN
LAGLKGRLDYLSSLKVKGLVLGPIHKNQKDDVAQTDLLQIDPNFGSKEDFDSLLOSAKKK
SIRVILDLTPNYRGENSWFSTQVDTVATKVKDALEFWLQAGVDGFQVRDIENLKDASSFL
AEWQNITKGFSEDRLLIAGTNSSDLQQILSLLESNKDLLLTSSYLSDSGSTGEHTKSLVT
QYLNATGNRWCSWSLSQARLLTSFLPAQLLRLYQLMLFTLPGTPVFSYGDEIGLDAAALP
GQPMEAPVMLWDESSFPDIPGAVSANMTVKGQSEDPGSLLSLFRRLSDORSKERSLLHGD
FHAFSAGPGLFSYIRHWDQNERFLVVLNFGDVGLSAGLQASDLPASASLPAKADLLLSTQ
PGREEGSPLELERLKLEPHEGLLLRFPYAA
SEQ ID NO: 5
Consensus Coding Sequence: CCDS 31588.1: RefSeq: NM_001012662.3,
ATGGAGCTACAGCCTCCTGAAGCCTCGATCGCCGTCGTGTCGATTCCGCGCCAGTTGCCTGGCTCACA
TTCGGAGGCTGGTGTCCAGGGTCTCAGCGCGGGGGACGACTCAGAGACGGGGTCTGACTGTGTTACCC
AGGCTGGTCTTCAACTCTTGGCCTCAAGTGATCCTCCTGCCTTAGCTTCCAAGAATGCTGAGGTTACA
GTAGAAACGGGGTTTCACCATGTTAGCCAGGCTGATATTGAATTCCTGACCTCAATTGATCCGACTGC
CTCGGCCTCCGGAAGTGCTGGGATTACAGGCACCATGAGCCAGGACACCGAGGTGGATATGAAGGAGG
TGGAGCTGAATGAGTTAGAGCCCGAGAAGCAGCCGATGAACGCGGCGTCTGGGGCGGCCATGTCCCTG
GCGGGAGCCGAGAAGAATGGTCTGGTGAAGATCAAGGTGGCGGAAGACGAGGCGGAGGCGGCAGCCGC
GGCTAAGTTCACGGGCCTGTCCAAGGAGGAGCTGCTGAAGGTGGCAGGCAGCCCCGGCTGGGTACGCA
CCCGCTGGGCACTGCTGCTGCTCTTCTGGCTCGGCTGGCTCGGCATGCTTGCTGGTGCCGTGGTCATA
ATCGTGCGAGCGCCGCGTTGTCGCGAGCTACCGGCGCAGAAGTGGTGGCACACGGGCGCCCTCTACCG
CATCGGCGACCTTCAGGCCTTCCAGGGCCACGGCGCGGGCAACCTGGCGGGTCTGAAGGGGCGTCTCG
ATTACCTGAGCTCTCTGAAGGTGAAGGGCCTTGTGCTGGGTCCAATTCACAAGAACCAGAAGGATGAT
GTCGCTCAGACTGACTTGCTGCAGATCGACCCCAATTTTGGCTCCAAGGAAGATTTTGACAGTCTCTT
GCAATCGGCTAAAAAAAAGAGCATCCGTGTCATTCTGGACCTTACTCCCAACTACCGGGGTGAGAACT
CGTGGTTCTCCACTCAGGTTGACACTGTGGCCACCAAGGTGAAGGATGCTCTGGAGTTTTGGCTGCAA
GCTGGCGTGGATGGGTTCCAGGTTCGGGACATAGAGAATCTGAAGGATGCATCCTCATTCTTGGCTGA
GTGGCAAAATATCACCAAGGGCTTCAGTGAAGACAGGCTCTTGATTGCGGGGACTAACTCCTCCGACC
TTCAGCAGATCCTGAGCCTACTCGAATCCAACAAAGACTTGCTGTTGACTAGCTCATACCTGTCTGAT
TCTGGTTCTACTGGGGAGCATACAAAATCCCTAGTCACACAGTATTTGAATGCCACTGGCAATCGCTG
GTGCAGCTGGAGTTTGTCTCAGGCAAGGCTCCTGACTTCCTTCTTGCCGGCTCAACTTCTCCGACTCT
ACCAGCTGATGCTCTTCACCCTGCCAGGGACCCCTGTTTTCAGCTACGGGGATGAGATTGGCCTGGAT
GCAGCTGCCCTTCCTGGACAGCCTATGGAGGCTCCAGTCATGCTGTGGGATGAGTCCAGCTTCCCTGA
CATCCCAGGGGCTGTAAGTGCCAACATGACTGTGAAGGGCCAGAGTGAAGACCCTGGCTCCCTCCTTT
CCTTGTTCCGGCGGCTGAGTGACCAGCGGAGTAAGGAGCGCTCCCTACTGCATGGGGACTTCCACGCG
TTCTCCGCTGGGCCTGGACTCTTCTCCTATATCCGCCACTGGGACCAGAATGAGCGTTTTCTGGTAGT
GCTTAACTTTGGGGATGTGGGCCTCTCGGCTGGACTGCAGGCCTCCGACCTGCCTGCCAGCGCCAGCC
TGCCAGCCAAGGCTGACCTCCTGCTCAGCACCCAGCCAGGCCGTGAGGAGGGCTCCCCTCTTGAGCTG
GAACGCCTGAAACTGGAGCCTCACGAAGGGCTGCTGCTCCGCTTCCCCTACGCGGCCTGA
SEQ ID NO: 6
xCT (SLC7A11)
Protein: UnitProt: Q9UPY5,
>sp|Q9UPY5|XCT_HUMAN Cystine/glutamate transporter OS = Homo sapiens OX = 9606
GN = SLC7A11 PE = 1 SV = 1
MVRKPVVSTISKGGYLQGNVNGRLPSLGNKEPPGQEKVQLKRKVTLLRGVSIIIGTIIGA
GIFISPKGVLQNTGSVGMSLTIWTVCGVLSLFGALSYAELGTTIKKSGGHYTYILEVFGP
LPAFVRVWVELLIIRPAATAVISLAFGRYILEPFFIQCEIPELAIKLITAVGITVVMVLN
SMSVSWSARIQIFLTFCKLTAILIIIVPGVMQLIKGQTONFKDAFSGRDSSITRLPLAFY
YGMYAYAGWFYLNFVTEEVENPEKTIPLAICISMAIVTIGYVLTNVAYFTTINAEELLLS
NAVAVTFSERLLGNFSLAVPIFVALSCFGSMNGGVFAVSRLFYVASREGHLPEILSMIHV
RKHTPLPAVIVLHPLTMIMLFSGDLDSLLNELSFARWLFIGLAVAGLIYLRYKCPDMHRP
FKVPLFIPALFSFTCLFMVALSLYSDPFSTGIGFVITLTGVPAYYLFIIWDKKPRWFRIM
SEKITRTLQIILEVVPEEDKL
SEQ ID NO: 7
Consensus Coding Sequence: CCDS3742.1: RefSeq: NM_014331.4,
ATGGTCAGAAAGCCTGTTGTGTCCACCATCTCCAAAGGAGGTTACCTGCAGGGAAATGTTAACGGGAG
GCTGCCTTCCCTGGGCAACAAGGAGCCACCTGGGCAGGAGAAAGTGCAGCTGAAGAGGAAAGTCACTT
TACTGAGGGGAGTCTCCATTATCATTGGCACCATCATTGGAGCAGGAATCTTCATCTCTCCTAAGGGC
GTGCTCCAGAACACGGGCAGCGTGGGCATGTCTCTGACCATCTGGACGGTGTGTGGGGTCCTGTCACT
ATTTGGAGCTTTGTCTTATGCTGAATTGGGAACAACTATAAAGAAATCTGGAGGTCATTACACATATA
TTTTGGAAGTCTTTGGTCCATTACCAGCTTTTGTACGAGTCTGGGTGGAACTCCTCATAATACGCCCT
GCAGCTACTGCTGTGATATCCCTGGCATTTGGACGCTACATTCTGGAACCATTTTTTATTCAATGTGA
AATCCCTGAACTTGCGATCAAGCTCATTACAGCTGTGGGCATAACTGTAGTGATGGTCCTAAATAGCA
TGAGTGTCAGCTGGAGCGCCCGGATCCAGATTTTCTTAACCTTTTGCAAGCTCACAGCAATTCTGATA
ATTATAGTCCCTGGAGTTATGCAGCTAATTAAAGGTCAAACGCAGAACTTTAAAGACGCCTTTTCAGG
AAGAGATTCAAGTATTACGCGGTTGCCACTGGCTTTTTATTATGGAATGTATGCATATGCTGGCTGGT
TTTACCTCAACTTTGTTACTGAAGAAGTAGAAAACCCTGAAAAAACCATTCCCCTTGCAATATGTATA
TCCATGGCCATTGTCACCATTGGCTATGTGCTGACAAATGTGGCCTACTTTACGACCATTAATGCTGA
GGAGCTGCTGCTTTCAAATGCAGTGGCAGTGACCTTTTCTGAGCGGCTACTGGGAAATTTCTCATTAG
CAGTTCCGATCTTTGTTGCCCTCTCCTGCTTTGGCTCCATGAACGGTGGTGTGTTTGCTGTCTCCAGG
TTATTCTATGTTGCGTCTCGAGAGGGTCACCTTCCAGAAATCCTCTCCATGATTCATGTCCGCAAGCA
CACTCCTCTACCAGCTGTTATTGTTTTGCACCCTTTGACAATGATAATGCTCTTCTCTGGAGACCTCG
ACAGTCTTTTGAATTTCCTCAGTTTTGCCAGGTGGCTTTTTATTGGGCTGGCAGTTGCTGGGCTGATT
TATCTTCGATACAAATGCCCAGATATGCATCGTCCTTTCAAGGTGCCACTGTTCATCCCAGCTTTGTT
TTCCTTCACATGCCTCTTCATGGTTGCCCTTTCCCTCTATTCGGACCCATTTAGTACAGGGATTGGCT
TCGTCATCACTCTGACTGGAGTCCCTGCGTATTATCTCTTTATTATATGGGACAAGAAACCCAGGTGG
TTTAGAATAATGTCGGAGAAAATAACCAGAACATTACAAATAATACTGGAAGTTGTACCAGAAGAAGA
TAAGTTATGA
SEQ ID NO: 8
P2A CLEAVAGE SEQUENCE
GCCACCAACTTCAGCCTGCTGAAGCAGGCCGGCGACGTGGAGGAAAACCCTGGCCCC
SEQ ID NO: 9
Sequence ID NO: 10-Amino acid sequence of exemplary CAR targeting GD2
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPDILLTQTPLSLPVSLGDQASISCRSSQSLVHRNGNTYLHWYLQKPGQ
SPKLLIHKVSNRFSGVPDRFSGSGSGTDFTLKISRVEAEDLGVYFCSQSTHVPPLTFGAGTKLELKRA
DAAPTVSIFPGSGGGGSGGEVKLQQSGPSLVEPGASVMISCKASGSSFTGYNMNWVRQNIGKSLE
WIGAIDPYYGGTSYNQKFKGRATLTVDKSSSTAYMHLKSLTSEDSAVYYCVSGMEYWGQGTSVTVS
SAKTTPPSVYGRVTVSSAEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKKDPKTTTPAPRPPTPAPTI
ASQPLSLRPEACRPAAGGAVHTRGLDFACDIYIWAPLAGTCGVLLLSLVITLYCKRGRKKLLYIFKQPF
MRPVQTTQEEDGCSCRFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDGLYQGLSTATKDTYDA
LHMQALPPRGSGATNFSLLKQAGDVEENPGP
Sequence ID NO: 11-Amino acid sequence of exemplary CAR targeting CD33
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPGSNIMLTQSPSSLAVSAGEKVTMSCKSSQSVFFSSSQKNYLAWYQQI
PGQSPKLLIYWASTRESGVPDRFTGSGSGTDFTLTISSVQSEDLAIYYCHQYLSSRTFGGGTKLEIKR
GGGGSGGGGSSGGGSQVQLQQPGAEVVKPGASVKMSCKASGYTFTSYYIHWIKQTPGQGLEWVG
VIYPGNDDISYNQKFKGKATLTADKSSTTAYMQLSSLTSEDSAVYYCAREVRLRYFDVWGAGTTVTV
SSTTTPAPRPPTPAPTIASQPLSLRPEACRPAAGGAVHTRGLDFACDIYIWAPLAGTCGVLLLSLVITLY
CKRGRKKLLYIFKQPFMRPVQTTQEEDGCSCRFPEEEEGGCELRVKFSRSADAPAYKQGQNQLYNE
LNLGRREEYDVLDKRRGRDPEMGGKPRRKNPQEGLYNELQKDKMAEAYSEIGMKGERRRGKGHD
GLYQGLSTATKDTYDALHMQALPPRGSGATNFSLLKQAGDVEENPGP
Sequence ID NO: 12-Amino acid sequence of exemplary CAR targeting mesothelin
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPMQVQLQQSGPELEKPGASVKISCKASGYSFTGYTMNWVKQSHGKSL
EWIGLITPYNGASSYNQKFRGKATLTVDKSSSTAYMDLLSLTSEDSAVYFCARGGYDGRGFDYWGQ
GTTVTVSSGVGGSGGGGSGGGGSDIELTQSPAIMSASPGEKVTMTCSASSSVSYMHWYQQKSGTS
PKRWIYDTSKLASGVPGRFSGSGSGNSYSLTISSVEAEDDATYYCQQWSGYPLTFGAGTKLEIKTTT
PAPRPPTPAPTIASQPLSLRPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQ
GLSTATKDTYDALHMQALPPRGSGATNFSLLKQAGDVEENPGP
Sequence ID NO: 13-Amino acid sequence of exemplary CAR targeting EGFR
MPRGWTALCLLSLLPSGFMSLDNNGTATPELPTQGTFSNVSTNVSYQETTTPSTLGSTSLHPVSQHG
NEATTNITETTVKFTSTSVITSVYGNTNSSVQSQTSVISTVFTTPANVSTPETTLKPSLSPGNVSDLSTT
STSLATSPTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVASHQSYS
QKTLIALVTSGALLAVLGITGYFLMNRRSWSPTGERLELEPVDRVKQTLNFDLLKLAGDVESNPGPGN
MALPVTALLLPLALLLHAARPQVQLQQSGGGLVKPGASLKLSCVTSGFTFRKFGMSWVRQTSDKRLE
WASISTGGYNTYYSDNVKGRFTISRENAKNTLYLQMSSLKSEDTALYYCTRGYSSTSYAMDYWGQ
GTTVTVSSSGGGSGGGGSGGGGSDIELTQSPASLSVATGEKVTIRCMTSTDIDDDMNWYQQKPGEP
PKFLISEGNTLRGVPSRFSSSGTGTDFVFTIENTLSEDVGDYYCLQSFNVPLTFGDGTKLEKALEQKLI
SEEDLAKPTTTPAPRPPTPAPTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCELRVKFSRSADAPAYKQGQ
NQLYNELNLGRREEYDVLDKRRGRDPEMGGKPRRKNPQEGLYNELQKDKMAEAYSEIGMKGERRR
GKGHDGLYQGLSTATKDTYDALHMQALPPRGSGATNFSLLKQAGDVEENPGP
Sequence ID NO: 14-Amino acid sequence of exemplary GD2 target binding moiety
DILLTQTPLSLPVSLGDQASISCRSSQSLVHRNGNTYLHWYLQKPGQSPKLLIHKVSNRFSGVPDRFS
GSGSGTDFTLKISRVEAEDLGVYFCSQSTHVPPLTFGAGTKLELKRADAAPTVSIFPGSGGGGSGGE
VKLQQSGPSLVEPGASVMISCKASGSSFTGYNMNWVRQNIGKSLEWIGAIDPYYGGTSYNQKFKGR
ATLTVDKSSSTAYMHLKSLTSEDSAVYYCVSGMEYWGQGTSVTVSSAKTTPPSVYGRVTVSS
Sequence ID NO: 15-Amino acid sequence of exemplary CD33 target binding moiety
GSNIMLTQSPSSLAVSAGEKVTMSCKSSQSVFFSSSQKNYLAWYQQIPGQSPKLLIYWASTRESGVP
DRFTGSGSGTDFTLTISSVQSEDLAIYYCHQYLSSRTFGGGTKLEIKRGGGGSGGGGSSGGGSQVQ
LQQPGAEVVKPGASVKMSCKASGYTFTSYYIHWIKQTPGQGLEWVGVIYPGNDDISYNQKFKGKATL
TADKSSTTAYMQLSSLTSEDSAVYYCAREVRLRYFDVWGAGTTVTVSS
Sequence ID NO: 16-Amino acid sequence of exemplary mesothelin target binding moiety
MQVQLQQSGPELEKPGASVKISCKASGYSFTGYTMNWVKQSHGKSLEWIGLITPYNGASSYNQKFR
GKATLTVDKSSSTAYMDLLSLTSEDSAVYFCARGGYDGRGFDYWGQGTTVTVSSGVGGSGGGGSG
GGGSDIELTQSPAIMSASPGEKVTMTCSASSSVSYMHWYQQKSGTSPKRWIYDTSKLASGVPGRFS
GSGSGNSYSLTISSVEAEDDATYYCQQWSGYPLTFGAGTKLEIK
Sequence ID NO: 17-Amino acid sequence of exemplary EGFRVIII target binding moiety
QVQLQQSGGGLVKPGASLKLSCVTSGFTFRKFGMSWVRQTSDKRLEWVASISTGGYNTYYSDNVK
GRFTISRENAKNTLYLQMSSLKSEDTALYYCTRGYSSTSYAMDYWGQGTTVTVSSSGGGSGGGGS
GGGGSDIELTQSPASLSVATGEKVTIRCMTSTDIDDDMNWYQQKPGEPPKFLISEGNTLRPGVPSRF
SSSGTGTDFVFTIENTLSEDVGDY
SEQ ID NO: 18, alternative EGFRvIII binding moiety encoded by SEQ ID NO: 19
MDWIWRILFLVGAATGAHSQVQLQQSGGGLVKPGASLKLSCVTSGFTFRKFGMSWVRQT
SDKRLEWVASISTGGYNTYYSDNVKGRFTISRENAKNTLYLQMSSLKSEDTALYYCTRGYS
STSYAMDYWGQGTTVTVSSSGGGSGGGGSGGGGSDIELTQSPASLSVATGEKVTIRCMT
STDIDDDMNWYQQKPGEPPKFLISEGNTLRPGVPSRFSSSGTGTDFVFTIENTLSEDVGDY
YCLQSFNVPLTFGDGTKLEKAL
Sequence ID No: 19-DNA encoding an alternative EGFRvlll target binding moiety
(EGFRvIII scFv sequence derived from MR1 antibody)
Atggactggatttggcgcatccttttccttgtcggcgctgctaccggcgcgcattctcaggtacaact
ccagcagtctgggggaggcttagtgaagcctggagcgtctctgaaactctcctgtgtaacctctggat
tcactttcagaaaatttggcatgtcttgggttcgccagactagtgacaagaggctggaatgggtcgca
tccattagtactggcggttataacacgtactattcagacaatgtaaagggccgattcaccatctccag
agagaatgccaagaacaccctgtacctgcaaatgagtagtctgaagtctgaggacacggccttgtatt
actgtacaagaggctattctagtacctcttatgctatggactactggggccaagggaccacggtcacc
gtctcctcaagtggaggcggttcaggcggaggtggctctggcggtggcggatcggacatcgagctcac
tcagtctccagcatccctgtccgtggctacaggagaaaaagtcactatcagatgcatgaccagcactg
atattgatgatgatatgaactggtaccagcagaagccaggggaaccccctaagttccttatttcagaa
ggcaatactcttcggccgggagtcccatcccgattttccagcagtggcactggcacagattttgtttt
tacaattgaaaacacactctcggaagatgttggagattactactgtttgcaaagctttaacgtgcctc
ttacattcggtgatggcaccaagcttgaaaaagctcta

Sequence ID No: 20—An exemplary nucleic acid construct of the invention encoding an anti-CD33 CAR (as fusion target-binding protein) and SLC7A5 (as amino acid transporter). Coding regions within the nucleic acid sequence of SEQ ID NO: 20 are identified as follows:

| | |
|---|---|
| tCD34 | “unformatted” text |
| F2A | Underlined bold text |
| CD8 signal peptide | Underlined italic text |
| CD33 ScFV | Underlined bold italic text |
| CD8 Hinge | Italic text |
| 4-1BB | Bold text |
| CD3z signalling domain | Double underlined italic text |
| P2A | Double underlined bold text |
| LAT1 (SLC7A5) | Bold italic text |

ATGCCTCGCGGCTGGACAGCCCTGTGCCTGCTGTCTCTGCTGCCATCCGGCTTCATGAGCCT
GGATAATAACGGCACAGCCACCCCAGAGCTGCCTACACAGGGCACCTTCAGCAATGTGTCCA
CAAACGTGAGCTATCAGGAGACCACAACCCCTTCTACCCTGGGATCCACAAGCCTGCACCCC
GTGTCTCAGCACGGCAACGAAGCCACCACCAACATCACCGAGACCACAGTGAAGTTTACCTC
CACCTCTGTGATTACCTCTGTGTACGGAAATACAAACTCCAGCGTGCAGTCTCAGACATCTG
TGATCTCCACAGTGTTTACAACACCTGCCAATGTGTCCACCCCAGAGACAACCCTGAAGCCC
AGCCTGTCTCCTGGAAATGTGTCCGATCTGTCTACCACCTCCACCAGCCTGGCCACCTCTCC
CACCAAGCCCTATACCTCCTCTTCTCCCATCCTGAGCGATATCAAAGCCGAGATCAAATGCA
GCGGGATTCGGGAAGTGAAACTGACACAGGGCATCTGCCTGGAACAGAATAAGACATCCAGC
TGCGCCGAGTTTAAGAAAGATAGAGGAGAGGGACTGGCCAGGGTGCTGTGTGGCGAAGAGCA
GGCCGACGCCGATGCCGGCGCCCAGGTGTGTTCCCTGCTGCTGGCCCAGTCTGAGGTGCGCC
CCCAGTGCCTGCTGCTGGTGCTGGCCAATCGGACAGAAATTAGCAGCAAGCTGCAGCTGATG
AAAAAACACCAGAGCGATCTGAAAAAGCTGGGCATCCTGGACTTTACCGAGCAGGACGTGGC
CTCTCACCAGAGCTACAGCCAGAAAACACTGATCGCCCTGGTGACCAGCGGAGCCCTGCTGG
CCGTGCTGGGCATCACCGGATATTTCCTGATGAATAGGCGCAGCTGGAGCCCCACCGGCGAA
CGGCTGGAGCTGGAGCCTGTCGACCGAGTGAAGCAGACCCTGAACTTTGATCTGCTGAAGCT
GGCCGGCGACGTGGAGTCCAACCCCGGGCCAGGGAATATGGCCTTACCAGTGACCGCCTTGC
TCCTGCCGCTGGCCTTGCTGCTCCACGCCGCCAGGCCG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ACCACGACGCCAGCACCGCGACCA
CCAACACCGGCGCCAACCATCGCATCGCAGCCCCTGTCCCTGCGCCCAGAGGCGTGCCGACC
AGCGGCGGGGGGCGCAGTGCACACGAGGGGGCTGGACTTCGCCTGTGATATCTACATCTGGG
CGCCCTTGGCCGGGACTTGTGGGGTCCTTCTCCTGTCACTGGTTATCACCCTTTACTGCAAA
CGGGGCAGAAAGAAACTCCTGTATATATTCAAACAACCATTTATGAGACCAGTACAAACTAC
TCAAGAGGAAGATGGCTGTAGCTGCCGATTTCCAGAAGAAGAAGAAGGAGGATGTGAACTG

[symbol]

[symbol]

[symbol]

[symbol]

[symbol]

[symbol]

[symbol]

[symbol]

[symbol]

[symbol]

[symbol]

[symbol]

[symbol]

TGTACA

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Sequence ID No: 21—an exemplary polypeptide sequence of the invention—the amino acid sequence produced on expression of the nucleic acids construct of Sequence ID No: 20. The polypeptide is subsequently cleaved at F2A and P2A to yield a separate anti-CD33 CAR and SLC7A5 amino acid transporter.

| | |
|---|---|
| tCD34 | “unformatted” text |
| F2A | Underlined bold text |
| Anti-CD33 CAR | Underlined bold italic text |
| P2A | Double underlined bold text |
| LAT1 (SLC7A5) | Bold italic text |

MPRGWTALCLLSLLPSGEMSLDNNGTATPELPTQGTFSNVSTNVSYQETTTPSTLGSTSLHP
VSQHGNEATTNITETTVKFTSTSVITSVYGNTNSSVQSQTSVISTVETTPANVSTPETTLKP
SLSPGNVSDLSTTSTSLATSPTKPYTSSSPILSDIKAEIKCSGIREVKLTQGICLEQNKTSS
CAEFKKDRGEGLARVLCGEEQADADAGAQVCSLLLAQSEVRPQCLLLVLANRTEISSKLQLM
KKHQSDLKKLGILDFTEQDVASHQSYSQKTLIALVTSGALLAVLGITGYFLMNRRSWSPTGE
RLELEPVDRVKQTLNFDLLKLAGDVESNPGP

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Sequence ID No: 22—An exemplary nucleic acid construct of the invention encoding an anti-CD33 CAR (as fusion target-binding protein) and SLC7A11 (as amino acid transporter). Coding regions within the nucleic acid sequence of SEQ ID NO: 20 are identified as follows:

| | |
|---|---|
| tCD34 | “unformatted” text |
| F2A | Underlined bold text |
| CD8 signal peptide | Underlined italic text |
| CD33 ScFV | Underlined bold italic text |
| CD8 Hinge | Italic text |
| 4-1BB | Bold text |
| CD3z signalling domain | Double underlined italic text |
| P2A | Double underlined bold text |
| xCT (SLC7A11) | Bold italic text |

ATGCCTCGCGGCTGGACAGCCCTGTGCCTGCTGTCTCTGCTGCCATCCGGCTTCATGAGCCT
GGATAATAACGGCACAGCCACCCCAGAGCTGCCTACACAGGGCACCTTCAGCAATGTGTCCA
CAAACGTGAGCTATCAGGAGACCACAACCCCTTCTACCCTGGGATCCACAAGCCTGCACCCC
GTGTCTCAGCACGGCAACGAAGCCACCACCAACATCACCGAGACCACAGTGAAGTTTACCTC
CACCTCTGTGATTACCTCTGTGTACGGAAATACAAACTCCAGCGTGCAGTCTCAGACATCTG
TGATCTCCACAGTGTTTACAACACCTGCCAATGTGTCCACCCCAGAGACAACCCTGAAGCCC
AGCCTGTCTCCTGGAAATGTGTCCGATCTGTCTACCACCTCCACCAGCCTGGCCACCTCTCC
CACCAAGCCCTATACCTCCTCTTCTCCCATCCTGAGCGATATCAAAGCCGAGATCAAATGCA
GCGGGATTCGGGAAGTGAAACTGACACAGGGCATCTGCCTGGAACAGAATAAGACATCCAGC
TGCGCCGAGTTTAAGAAAGATAGAGGAGAGGGACTGGCCAGGGTGCTGTGTGGCGAAGAGCA
GGCCGACGCCGATGCCGGCGCCCAGGTGTGTTCCCTGCTGCTGGCCCAGTCTGAGGTGCGCC
CCCAGTGCCTGCTGCTGGTGCTGGCCAATCGGACAGAAATTAGCAGCAAGCTGCAGCTGATG
AAAAAACACCAGAGCGATCTGAAAAAGCTGGGCATCCTGGACTTTACCGAGCAGGACGTGGC
CTCTCACCAGAGCTACAGCCAGAAAACACTGATCGCCCTGGTGACCAGCGGAGCCCTGCTGG
CCGTGCTGGGCATCACCGGATATTTCCTGATGAATAGGCGCAGCTGGAGCCCCACCGGCGAA
CGGCTGGAGCTGGAGCCTGTCGACCGAGTGAAGCAGACCCTGAACTTTGATCTGCTGAAGCT
GGCCGGCGACGTGGAGTCCAACCCCGGGCCAGGGAATATGGCCTTACCAGTGACCGCCTTGC
TCCTGCCGCTGGCCTTGCTGCTCCACGCCGCCAGGCCG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ACCACGACGCCAGCACCGCGACCA
CCAACACCGGCGCCAACCATCGCATCGCAGCCCCTGTCCCTGCGCCCAGAGGCGTGCCGACC
AGCGGCGGGGGGCGCAGTGCACACGAGGGGGCTGGACTTCGCCTGTGATATCTACATCTGGG
CGCCCTTGGCCGGGACTTGTGGGGTCCTTCTCCTGTCACTGGTTATCACCCTTTACTGCAAA
CGGGGCAGAAAGAAACTCCTGTATATATTCAAACAACCATTTATGAGACCAGTACAAACTAC
TCAAGAGGAAGATGGCTGTAGCTGCCGATTTCCAGAAGAAGAAGAAGGAGGATGTGAACTG

[symbol]

[symbol]

[symbol]

[symbol]

[symbol]

[symbol]

[symbol]

[symbol]

[symbol]

[symbol]

TGTACA

[symbol]

[symbol]

[symbol]

[symbol]

[symbol]

[symbol]

[symbol]

[symbol]

[symbol]

[symbol]

[symbol]

GCCACCACCTTCAGCCTGCTGAAGCAGGCCGGCGACG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Sequence ID No: 23—an exemplary polypeptide sequence of the invention—the amino acid sequence produced on expression of the nucleic acids construct of Sequence ID No: 21. The polypeptide is subsequently cleaved at F2A and P2A to yield a separate anti-CD33 CAR and SLC7A11 amino acid transporter.

| | |
|---|---|
| tCD34 | “unformatted” text |
| F2A | Underlined bold text |
| Anti-CD33 CAR | Underlined bold italic text |
| P2A | Double underlined bold text |
| xCT (SLC7A11) | Bold italic text |

MPRGWTALCLLSLLPSGFMSLDNNGTATPELPTQGTFSNVSTNVSYQETTTPSTLGSTSLHP
VSQHGNEATTNITETTVKFTSTSVITSVYGNTNSSVQSQTSVISTVETTPANVSTPETTLKP
SLSPGNVSDLSTTSTSLATSPTKPYTSSSPILSDIKAEIKCSGIREVKLTQGICLEQNKTSS
CAEFKKDRGEGLARVLCGEEQADADAGAQVCSLLLAQSEVRPQCLLLVLANRTEISSKLQLM
KKHQSDLKKLGILDFTEQDVASHQSYSQKTLIALVTSGALLAVLGITGYFLMNRRSWSPTGE
RLELEPVDRVKQTLNFDLLKLAGDVESNPGP

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ATNFSLLKQAGDVEENPGP

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

The following paragraphs are not claims, but represent preferred aspects and embodiments of the invention.

1. A nucleic acid construct comprising a first sequence encoding an amino acid transporter, and a second sequence encoding a fusion target-binding protein.

2. A nucleic acid construct according to paragraph 1, wherein the fusion target-binding protein is a chimeric antigen receptor (CAR).

3. A nucleic acid construct according to paragraph 1 or paragraph 2, comprising a third sequence encoding a CD98 chain.

4. A nucleic acid construct according to any preceding paragraph, comprising one or more further sequences encoding one or more further amino acid transporters.

5. A nucleic acid construct according to any preceding paragraph, wherein the amino acid transporter encoded by the first sequence, and the further amino acid transporter encoded by the further sequence if present, is selected from the group consisting of: the SLC7A5 gene product, or a fragment or variant thereof; the SLC7A11 gene product, or a fragment or variant thereof; and the SLC7A1 gene product, or a fragment or variant thereof; or the gene product, or a fragment or derivative thereof, of any of the genes set out in Table 1.

6. A nucleic acid construct according to paragraph 5, comprising a sequence encoding the SLC7A5 gene product, or a fragment or derivative thereof.

7. A nucleic acid construct according to paragraph 5, comprising a sequence encoding the SLC7A11 gene product, or a fragment or derivative thereof.

8. A nucleic acid construct according to paragraph 5, comprising a sequence encoding the SLC7A1 gene product, or a fragment or derivative thereof.

9. A nucleic acid construct according to any preceding paragraph, wherein each of the first and second sequences, and the third and further sequences if present, are separated by a cleavage sequence located between the respective sequences.

10. A nucleic acid construct according to any preceding paragraph, wherein the fusion target-binding protein encoded by the second sequence is selected from the group consisting of: an anti-GD2 fusion target-binding protein; an anti-CD33 fusion target-binding protein; an anti-mesothelin fusion target-binding protein; an EGFRvIII fusion target-binding protein; an anti-VEGFR2 fusion target-binding protein; an anti-FAP fusion target-binding protein; an anti-EpCam fusion target-binding protein; an anti-GPC3 fusion target-binding protein; an anti-CD133 fusion target-binding protein; an anti-IL13Ra fusion target-binding protein; an anti-EphA2 fusion target-binding protein; an anti-Muc1 fusion target-binding protein; an anti-BCMA fusion target-binding protein; an anti-CD70 fusion target-binding protein; an anti-CD123 fusion target-binding protein; an anti-ROR1 fusion target-binding protein; an anti-PSMA fusion target-binding protein; an anti-CD5 fusion target-binding protein; an anti-GAP fusion target-binding protein; an anti-CEA fusion target-binding protein; an anti-PSCA fusion target-binding protein; an anti-Her2 fusion target-binding protein; and an anti-CD19 fusion target-binding protein.

11. A nucleic acid construct according to paragraph 10, wherein the anti-GD2 fusion target-binding protein encoded by the second sequence is an anti-GD2 CAR.

12. A nucleic acid construct according to paragraph 11, wherein the anti-GD2 CAR encoded by the second sequence comprises the CAR of SEQ ID NO: 10, or a fragment thereof, or a variant sharing at least 75% sequence identity with SEQ ID NO: 10 or a fragment thereof.

13. A nucleic acid construct according to paragraph 10, wherein the anti-CD33 fusion target-binding protein encoded by the second sequence is an anti-CD33 CAR.

14. A nucleic acid construct according to paragraph 13, wherein the anti-CD33 CAR encoded by the second sequence comprises the CAR of SEQ ID NO: 11, or a fragment thereof, or a variant sharing at least 75% sequence identity with SEQ ID NO: 11 or a fragment thereof.

15. A nucleic acid construct according to paragraph 10, wherein the anti-mesothelin fusion target-binding protein encoded by the second sequence is an anti-mesothelin CAR.

16. A nucleic acid construct according to paragraph 15, wherein the anti-mesothelin CAR encoded by the second sequence comprises the CAR of SEQ ID NO: 12, or a fragment thereof, or a variant sharing at least 75% sequence identity with SEQ ID NO: 12 or a fragment thereof.

17. A nucleic acid construct according to paragraph 10, wherein the anti-EGFRvIII fusion target-binding protein encoded by the second sequence is an anti-EGFRvIII CAR.

18. A nucleic acid construct according to paragraph 17, wherein the anti-EGFRvIII CAR encoded by the second sequence comprises the CAR of SEQ ID NO: 13, or a fragment thereof, or a variant sharing at least 75% sequence identity with SEQ ID NO: 13 or a fragment thereof.

19. A nucleic acid construct according to any preceding paragraph, in the form of a vector.

20. A nucleic acid sequence according to paragraph 19, wherein the vector is selected from the group consisting of: a retrovirus vector: and a lentivirus vector.

21. A cell comprising a nucleic acid construct according to any preceding paragraph.

22. A method of manufacturing a therapeutically modified cell, the method comprising providing a cell with a nucleic acid sequence encoding a fusion target-binding protein and a nucleic acid sequence encoding an amino acid transporter such that the nucleic acid sequences are expressed by the cell to produce an amino acid transporter and a fusion target-binding protein.

23. A method of manufacturing a therapeutically modified cell according to paragraph 22, the method comprising providing a cell with a nucleic acid construct according to any of paragraphs 1 to 20, such that the nucleic acid construct is expressed by the cell to produce an amino acid transporter and a fusion target-binding protein.

24. A cell according to paragraph 21, or a method of manufacturing a therapeutically modified cell according to paragraph 22 or 23, wherein the cell is selected from the group consisting of: a T cell; and a natural killer (NK) cell.

25. A cell according to paragraph 21 or paragraph 24, for use in the prevention and/or treatment of a disease such as cancer.

1. A cell comprising a nucleic acid construct, wherein the nucleic acid construct comprises a first sequence encoding an amino acid transporter, and a second sequence encoding a fusion target-binding protein, wherein the amino acid transporter encoded by the first sequence, is selected from the group consisting of: the SLC7A5 gene product, or a fragment or variant thereof; the SLC7A11 gene product, or a fragment or variant thereof; and
the SLC7A1 gene product, or a fragment or variant thereof; or the gene product, or a fragment or derivative thereof, of any of the genes set out in Table 1, and wherein the cell demonstrates increased proliferation in the presence of cancer cells expressing a target protein recognised by the fusion target-binding protein, compared to a comparator CAR T cell.

2. A cell according to claim 1, wherein the fusion target-binding protein is a chimeric antigen receptor (CAR).

3. A cell according to claim 1 or claim 2, wherein the nucleic acid construct comprises a third sequence encoding a CD98 chain.

4. A cell according to any preceding claim, wherein the nucleic acid construct comprises one or more further sequences encoding one or more further amino acid transporters.

5. A cell according to any preceding claim, wherein the nucleic acid construct comprises a sequence encoding the SLC7A5 gene product, or a fragment or derivative thereof.

6. A cell according to any preceding claim, wherein the nucleic acid construct comprises a sequence encoding the SLC7A11 gene product, or a fragment or derivative thereof.

7. A cell according to any preceding claim, wherein the nucleic acid construct comprises a sequence encoding the SLC7A1 gene product, or a fragment or derivative thereof.

8. A cell according to any preceding claim, wherein each of the first and second sequences, and the third and further sequences if present of the nucleic acid construct, are separated by a cleavage sequence located between the respective sequences.

9. A cell according to any preceding claim, wherein the fusion target-binding protein encoded by the second sequence is selected from the group consisting of: an anti-GD2 fusion target-binding protein; an anti-CD33 fusion target-binding protein; an anti-mesothelin fusion target-binding protein; an EGFRvIII fusion target-binding protein; an anti-VEGFR2 fusion target-binding protein; an anti-FAP fusion target-binding protein; an anti-EpCam fusion target-binding protein; an anti-GPC3 fusion target-binding protein; an anti-CD133 fusion target-binding protein; an anti-IL13Ra fusion target-binding protein; an anti-EphA2 fusion target-binding protein; an anti-Muc1 fusion target-binding protein; an anti-BCMA fusion target-binding protein; an anti-CD70 fusion target-binding protein; an anti-CD123 fusion target-binding protein; an anti-ROR1 fusion target-binding protein; an anti-PSMA fusion target-binding protein; an anti-CD5 fusion target-binding protein; an anti-GAP fusion target-binding protein; an anti-CEA fusion target-binding protein; an anti-PSCA fusion target-binding protein; an anti-Her2 fusion target-binding protein; and an anti-CD19 fusion target-binding protein.

10. A cell according to claim 9, wherein the anti-GD2 fusion target-binding protein encoded by the second sequence is an anti-GD2 CAR.

11. A cell according to claim 10, wherein the anti-GD2 CAR encoded by the second sequence comprises the CAR of SEQ ID NO: 10, or a fragment thereof, or a variant sharing at least 75% sequence identity with SEQ ID NO: 10 or a fragment thereof.

12. A cell according to claim 9, wherein the anti-CD33 fusion target-binding protein encoded by the second sequence is an anti-CD33 CAR.

13. A cell according to claim 12, wherein the anti-CD33 CAR encoded by the second sequence comprises the CAR of SEQ ID NO: 11, or a fragment thereof, or a variant sharing at least 75% sequence identity with SEQ ID NO: 11 or a fragment thereof.

14. A cell according to claim 9, wherein the anti-mesothelin fusion target-binding protein encoded by the second sequence is an anti-mesothelin CAR.

15. A cell according to claim 14, wherein the anti-mesothelin CAR encoded by the second sequence comprises the CAR of SEQ ID NO: 12, or a fragment thereof, or a variant sharing at least 75% sequence identity with SEQ ID NO: 12 or a fragment thereof.

16. A cell according to claim 9, wherein the anti-EGFRvIII fusion target-binding protein encoded by the second sequence is an anti-EGFRvIII CAR.

17. A cell according to claim 16, wherein the anti-EGFRvIII CAR encoded by the second sequence comprises the CAR of SEQ ID NO: 13, or a fragment thereof, or a variant sharing at least 75% sequence identity with SEQ ID NO: 13 or a fragment thereof.

18. A method of manufacturing a therapeutically modified cell according to any of claims 1-17, the method comprising providing the cell with a nucleic acid sequence encoding the fusion target-binding protein and a nucleic acid sequence encoding the amino acid transporter such that the nucleic acid sequences are expressed by the cell to produce the amino acid transporter and a fusion target-binding protein.

19. A cell according to any of claims 1 to 17, or a method of manufacturing a therapeutically modified cell according to claim 18, wherein the cell is selected from the group consisting of: a T cell; and a natural killer (NK) cell.

20. A cell according to any of claims 1 to 17, or claim 19, for use in the prevention and/or treatment of a disease such as cancer.


SEQUENCE LISTING (23 sequences)

SEQ ID NO: 1 - protein, 629 aa, Homo sapiens
MGCKVLLNIGQQMLRRKVVDCSREETRLSRCLNTFDLVALGVGSTLGAGVYVLAGAVARENAGPAIVISFLIAALASVLAGLCYGEFGARVPKTGSAYLYSYVTVGELWAFITGWNLILSYIIGTSSVARAWSATFDELIGRPIGEFSRTHMTLNAPGVLAENPDIFAVIIILILTGLLTLGVKESAMVNKIFTCINVLVLGFIMVSGFVKGSVKNWQLTEEDFGNTSGRLCLNNDTKEGKPGVGGFMPFGFSGVLSGAATCFYAFVGFDCIATTGEEVKNPQKAIPVGIVASLLICFIAYFGVSAALTLMMPYFCLDNNSPLPDAFKHVGWEGAKYAVAVGSLCALSASLLGSMFPMPRVIYAMAEDGLLFKFLANVNDRTKTPIIATLASGAVAAVMAFLFDLKDLVDLMSIGTLLAYSLVAACVLVLRYQPEQPNLVYQMASTSDELDPADQNELASTNDSQLGFLPEAEMFSLKTILSPKNMEPSKISGLIVNISTSLIAVLIITFCIVTVLGREALTKGALWAVFLLAGSALLCAVVTGVIWRQPESKTKLSFKVPFLPVLPILSIFVNVYLMMQLDQGTWVRFAVWMLIGFIIYFGYGLWHSEEASLDADQARTPDGNLDQCK

SEQ ID NO: 2 - DNA, 1890 bp, Homo sapiens
atggggtgcaaagtcctgctcaacattgggcagcagatgctgcggcggaaggtggtggactgtagccgggaggagacgcggctgtctcgctgcctgaacacttttgatctggtggccctcggggtgggcagcacactgggtgctggtgtctacgtcctggctggagctgtggcccgtgagaatgcaggccctgccattgtcatctccttcctgatcgctgcgctggcctcagtgctggctggcctgtgctatggcgagtttggtgctcgggtccccaagacgggctcagcttacctctacagctatgtcaccgttggagagctctgggccttcatcaccggctggaacttaatcctctcctacatcatcggtacttcaagcgtagcgagggcctggagcgccaccttcgacgagctgataggcagacccatcggggagttctcacggacacacatgactctgaacgcccccggcgtgctggctgaaaaccccgacatattcgcagtgatcataattctcatcttgacaggacttttaactcttggtgtgaaagagtcggccatggtcaacaaaatattcacttgtattaacgtcctggtcctgggcttcataatggtgtcaggatttgtgaaaggatcggttaaaaactggcagctcacggaggaggattttgggaacacatcaggccgtctctgtttgaacaatgacacaaaagaagggaagcccggtgttggtggattcatgcccttcgggttctctggtgtcctgtcgggggcagcgacttgcttctatgccttcgtgggctttgactgcatcgccaccacaggtgaagaggtgaagaacccacagaaggccatccccgtggggatcgtggcgtccctcttgatctgcttcatcgcctactttggggtgtcggctgccctcacgctcatgatgccctacttctgcctggacaataacagccccctgcccgacgcctttaagcacgtgggctgggaaggtgccaagtacgcagtggccgtgggctccctctgcgctctttccgccagtcttctaggttccatgtttcccatgcctcgggttatctatgccatggctgaggatggactgctatttaaattcttagccaacgtcaatgataggaccaaaacaccaataatcgccacattagcctcgggtgccgttgctgctgtgatggccttcctctttgacctgaaggacttggtggacctcatgtccattggcactctcctggcttactcgttggtggctgcctgtgtgttggtcttacggtaccagccagagcagcctaacctggtataccagatggccagtacttccgacgagttagatccagcagaccaaaatgaattggcaagcaccaatgattcccagctgggctttttaccagaggcagagatgttctctttgaaaaccatactctcacccaaaaacatggagccttccaaaatctctgggctaattgtgaacatttcaaccagcctcatagctgttctcatcatcaccttctgcattgtgaccgtgcttggaagggaggctctcaccaaaggggcgctgtgggcagtctttctgctcgcagggtctgccctcctctgtgccgtggtcacgggcgtcatctggaggcagcccgagagcaagaccaagctctcatttaaggttcccttcctgccagtgctccccatcctgagcatcttcgtgaacgtctatctcatgatgcagctggaccagggcacctgggtccggtttgctgtgtggatgctgataggcttcatcatctactttggctatggcctgtggcacagcgaggaggcgtccctggatgccgaccaagcaaggactcctgacggcaacttggaccagtgcaagtga

SEQ ID NO: 3 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 4 - DNA, 1524 bp, Homo sapiens
atggcgggtgcgggcccgaagcggcgcgcgctagcggcgccggcggccgaggagaaggaagaggcgcgggagaagatgctggccgccaagagcgcggacggctcggcgccggcaggcgagggcgagggcgtgaccctgcagcggaacatcacgctgctcaacggcgtggccatcatcgtggggaccattatcggctcgggcatcttcgtgacgcccacgggcgtgctcaaggaggcaggctcgccggggctggcgctggtggtgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccacctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagacatag

SEQ ID NO: 5 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 6 - DNA, 1896 bp, Homo sapiens
atggagctacagcctcctgaagcctcgatcgccgtcgtgtcgattccgcgccagttgcctggctcacattcggaggctggtgtccagggtctcagcgcgggggacgactcagagacggggtctgactgtgttacccaggctggtcttcaactcttggcctcaagtgatcctcctgccttagcttccaagaatgctgaggttacagtagaaacggggtttcaccatgttagccaggctgatattgaattcctgacctcaattgatccgactgcctcggcctccggaagtgctgggattacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctga

SEQ ID NO: 7 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 8 - DNA, 1506 bp, Homo sapiens
atggtcagaaagcctgttgtgtccaccatctccaaaggaggttacctgcagggaaatgttaacgggaggctgccttccctgggcaacaaggagccacctgggcaggagaaagtgcagctgaagaggaaagtcactttactgaggggagtctccattatcattggcaccatcattggagcaggaatcttcatctctcctaagggcgtgctccagaacacgggcagcgtgggcatgtctctgaccatctggacggtgtgtggggtcctgtcactatttggagctttgtcttatgctgaattgggaacaactataaagaaatctggaggtcattacacatatattttggaagtctttggtccattaccagcttttgtacgagtctgggtggaactcctcataatacgccctgcagctactgctgtgatatccctggcatttggacgctacattctggaaccattttttattcaatgtgaaatccctgaacttgcgatcaagctcattacagctgtgggcataactgtagtgatggtcctaaatagcatgagtgtcagctggagcgcccggatccagattttcttaaccttttgcaagctcacagcaattctgataattatagtccctggagttatgcagctaattaaaggtcaaacgcagaactttaaagacgccttttcaggaagagattcaagtattacgcggttgccactggctttttattatggaatgtatgcatatgctggctggttttacctcaactttgttactgaagaagtagaaaaccctgaaaaaaccattccccttgcaatatgtatatccatggccattgtcaccattggctatgtgctgacaaatgtggcctactttacgaccattaatgctgaggagctgctgctttcaaatgcagtggcagtgaccttttctgagcggctactgggaaatttctcattagcagttccgatctttgttgccctctcctgctttggctccatgaacggtggtgtgtttgctgtctccaggttattctatgttgcgtctcgagagggtcaccttccagaaatcctctccatgattcatgtccgcaagcacactcctctaccagctgttattgttttgcaccctttgacaatgataatgctcttctctggagacctcgacagtcttttgaatttcctcagttttgccaggtggctttttattgggctggcagttgctgggctgatttatcttcgatacaaatgcccagatatgcatcgtcctttcaaggtgccactgttcatcccagctttgttttccttcacatgcctcttcatggttgccctttccctctattcggacccatttagtacagggattggcttcgtcatcactctgactggagtccctgcgtattatctctttattatatgggacaagaaacccaggtggtttagaataatgtcggagaaaataaccagaacattacaaataatactggaagttgtaccagaagaagataagttatga

SEQ ID NO: 9 - DNA, 57 bp, Artificial Sequence
  P2A CLEAVAGE SEQUENCE
gccaccaacttcagcctgctgaagcaggccggcgacgtggaggaaaaccctggcccc

SEQ ID NO: 10 - protein, 1109 aa, Artificial Sequence
  CAR targeting GD2
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 11 - protein, 857 aa, Artificial Sequence
  CAR targeting CD33
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 12 - protein, 850 aa, Artificial Sequence
  CAR targeting mesothelin
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPMQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 13 - protein, 864 aa, Artificial Sequence
  CAR targeting EGFR
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRGVPSRFSSSGTGTDFVFTIENTLSEDVGDYYCLQSFNVPLTFGDGTKLEKALEQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 14 - protein, 263 aa, Artificial Sequence
  GD2 target binding moiety
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSS

SEQ ID NO: 15 - protein, 248 aa, Artificial Sequence
  CD33 target binding moiety
GSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 16 - protein, 241 aa, Artificial Sequence
  mesothelin target binding moiety
MQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 17 - protein, 221 aa, Artificial Sequence
  EGFRVIII target binding moiety
QVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDY

SEQ ID NO: 18 - protein, 262 aa, Artificial Sequence
  alternative EGFRvIII binding moiety
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSSGGGSGGGGSGGGGSDIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKAL

SEQ ID NO: 19 - DNA, 786 bp, Artificial Sequence
  alternative EGFRvIII target binding moiety (EGFRvIII scFv sequence derived from MR1 antibody)
atggactggatttggcgcatccttttccttgtcggcgctgctaccggcgcgcattctcaggtacaactccagcagtctgggggaggcttagtgaagcctggagcgtctctgaaactctcctgtgtaacctctggattcactttcagaaaatttggcatgtcttgggttcgccagactagtgacaagaggctggaatgggtcgcatccattagtactggcggttataacacgtactattcagacaatgtaaagggccgattcaccatctccagagagaatgccaagaacaccctgtacctgcaaatgagtagtctgaagtctgaggacacggccttgtattactgtacaagaggctattctagtacctcttatgctatggactactggggccaagggaccacggtcaccgtctcctcaagtggaggcggttcaggcggaggtggctctggcggtggcggatcggacatcgagctcactcagtctccagcatccctgtccgtggctacaggagaaaaagtcactatcagatgcatgaccagcactgatattgatgatgatatgaactggtaccagcagaagccaggggaaccccctaagttccttatttcagaaggcaatactcttcggccgggagtcccatcccgattttccagcagtggcactggcacagattttgtttttacaattgaaaacacactctcggaagatgttggagattactactgtttgcaaagctttaacgtgcctcttacattcggtgatggcaccaagcttgaaaaagctcta

SEQ ID NO: 20 - DNA, 4086 bp, Artificial Sequence
  anti-CD33 CAR (as fusion target-binding protein) and SLC7A5 (as amino acid transporter)
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgaacggctggagctggagcctgtcgaccgagtgaagcagaccctgaactttgatctgctgaagctggccggcgacgtggagtccaaccccgggccagggaatatggccttaccagtgaccgccttgctcctgccgctggccttgctgctccacgccgccaggccgggatccaacatcatgctgacccagagccctagcagcctggccgtgtctgccggcgagaaagtgaccatgagctgcaagagcagccagagcgtgttcttcagcagctcccagaagaactacctagcctggtatcagcagatcccaggccagagccctaagctgctgatctactgggccagcaccagagaaagcggcgtgcccgatagattcaccggaagcggttctggcaccgacttcaccctgacaatcagcagcgtgcagagcgaggacctggccatctactactgccaccagtacctgagcagccggacctttggcggaggcaccaagctggaaatcaagagaggcggcggaggctcaggcggaggcggatctagtggcggaggatctcaggtgcagctgcagcagccaggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttcaagggcaaggccaccctgaccgccgacaagtctagcaccaccgcctacatgcagctgtccagcctgaccagcgaggacagcgccgtgtactactgcgccagagaagtgcggctgcggtacttcgatgtgtggggagccggcaccaccgtgaccgtgtcatctaccacgacgccagcaccgcgaccaccaacaccggcgccaaccatcgcatcgcagcccctgtccctgcgcccagaggcgtgccgaccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccaccaacttcagcctgctgaagcaggccggcgacgtggaggaaaaccctggccccatggccggcgctggacctaagagaagggctctggctgctcccgctgctgaggagaaagaagaggctagagagaagatgctggctgctaagagcgccgatggctccgctcccgccggagaaggagagggcgtgacactgcagaggaacatcacactgctcaacggcgtcgccattatcgtgggcaccatcattggaagcggcatcttcgtgacccccaccggcgtgctgaaggaggctggatcccccggactggctctggtggtctgggctgcttgcggcgtgttcagcattgtgggcgctctgtgttacgccgaactgggcaccaccattagcaaaagcggaggagactacgcctacatgctggaagtgtacggaagcctccccgccttcctcaagctgtggatcgagctgctgatcattaggccttccagccagtacatcgtcgccctcgtcttcgccacatatctgctcaagcctctgtttcccacatgccccgtgcccgaggaggctgctaaactggtcgcttgtctctgtgtgctgctgctgacagccgtcaattgttacagcgtcaaggctgccacaagagtgcaagacgcttttgccgccgctaagctgctcgccctcgctctgatcattctgctgggcttcgtccaaattggcaagggcgatgtcagcaatctggaccccaacttctccttcgagggcaccaaactcgacgtgggcaacatcgtgctcgctctgtattccggactgttcgcttacggaggctggaactatctgaacttcgtcaccgaggagatgatcaacccctatagaaatctgcctctggctatcatcatctctctgcccatcgtcacactggtgtatgtgctcaccaatctggcctacttcaccacactgagcacagaacagatgctgtccagcgaagccgtcgccgtggactttggcaactatcatctgggcgtgatgagctggatcatccccgtgttcgtcggactgagctgcttcggctccgtgaatggatccctctttacaagctctagactcttcttcgtgggctctagagagggacatctgcctagcatcctcagcatgattcaccctcagctgctgacccccgtgccctctctggtgttcacatgcgtgatgacactgctgtacgccttcagcaaggatatcttcagcgtgattaatttttttagctttttcaactggctctgcgtggctctggccatcattggcatgatttggctgagacatagaaaacccgagctggagaggcccatcaaagtgaatctggctctgcccgtgttcttcattctcgcttgtctgtttctgattgccgtgtccttctggaagacccccgtggaatgcggcattggcttcaccatcattctgtccggcctccccgtgtacttcttcggcgtctggtggaagaacaagcccaagtggctgctgcaaggcatcttcagcaccacagtgctgtgccaaaagctgatgcaagtggtgccccaagagacatga

SEQ ID NO: 21 - protein, 1361 aa, Artificial Sequence
  amino acid sequence produced on expression of the nucleic acids construct of Sequence ID No 20
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 22 - DNA, 4068 bp, Artificial Sequence
  anti-CD33 CAR (as fusion target-binding protein) and SLC7A11 (as amino acid transporter)
atgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatccacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgaacggctggagctggagcctgtcgaccgagtgaagcagaccctgaactttgatctgctgaagctggccggcgacgtggagtccaaccccgggccagggaatatggccttaccagtgaccgccttgctcctgccgctggccttgctgctccacgccgccaggccgggatccaacatcatgctgacccagagccctagcagcctggccgtgtctgccggcgagaaagtgaccatgagctgcaagagcagccagagcgtgttcttcagcagctcccagaagaactacctagcctggtatcagcagatcccaggccagagccctaagctgctgatctactgggccagcaccagagaaagcggcgtgcccgatagattcaccggaagcggttctggcaccgacttcaccctgacaatcagcagcgtgcagagcgaggacctggccatctactactgccaccagtacctgagcagccggacctttggcggaggcaccaagctggaaatcaagagaggcggcggaggctcaggcggaggcggatctagtggcggaggatctcaggtgcagctgcagcagccaggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttcaagggcaaggccaccctgaccgccgacaagtctagcaccaccgcctacatgcagctgtccagcctgaccagcgaggacagcgccgtgtactactgcgccagagaagtgcggctgcggtacttcgatgtgtggggagccggcaccaccgtgaccgtgtcatctaccacgacgccagcaccgcgaccaccaacaccggcgccaaccatcgcatcgcagcccctgtccctgcgcccagaggcgtgccgaccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccaccaacttcagcctgctgaagcaggccggcgacgtggaggaaaaccctggccccatggtgaggaaacccgtggtgtccacaatcagcaagggcggctatctgcaaggcaatgtgaacggcagactgcctagcctcggaaacaaggaaccccccggccaagagaaggtgcagctgaagaggaaagtgacactgctgagaggcgtctccatcattatcggcaccatcatcggagctggcatcttcatcagccctaagggcgtgctccagaacaccggatccgtcggcatgtctctgaccatctggaccgtctgcggcgtgctgtctctgtttggcgccctctcctacgccgagctgggaacaaccatcaagaagtccggcggccactatacctacattctggaagtcttcggccctctgcccgcctttgtgagagtgtgggtggagctgctgatcattagacccgccgccaccgctgtcatttctctggctttcggaaggtacattctggagcccttcttcatccagtgtgagatccccgagctggctatcaagctgatcaccgctgtcggcatcaccgtggtcatggtcctcaacagcatgagcgtgagctggagcgctaggatccagatctttctgaccttctgcaagctgacagccattctgatcatcattgtccccggagtcatgcagctgattaagggccagacccagaactttaaggacgcctttagcggaagagacagcagcatcacaaggctgcccctcgccttttactacggcatgtacgcctatgccggctggttttatctgaacttcgtcaccgaggaggtggagaaccccgagaagaccatccctctggccatctgcatctccatggccatcgtgacaatcggatacgtgctcaccaacgtcgcctactttaccaccatcaatgccgaagagctgctgctctccaatgctgtcgccgtcacattttccgaaaggctgctgggcaatttctctctggccgtgcctatctttgtggctctgagctgtttcggcagcatgaacggcggagtgttcgccgtgtctagactgttctacgtggcctccagagagggacatctgcccgagatcctctccatgatccacgtgaggaagcacacccctctgcccgccgtgatcgtgctccaccctctgaccatgatcatgctcttttccggagatctggattctctgctgaactttctgtccttcgctagatggctgtttatcggactggctgtggccggactgatttatctgagatacaagtgccccgacatgcacagaccctttaaggtgcctctgtttatccccgctctgttcagcttcacatgtctgttcatggtcgccctctccctctacagcgaccccttctccaccggcatcggattcgtcattacactgaccggcgtgcccgcctattacctctttatcatctgggacaagaagcctagatggtttaggatcatgagcgagaagatcacaagaaccctccaaatcattctcgaggtggtccccgaggaagataagctgtga

SEQ ID NO: 23 - protein, 1355 aa, Artificial Sequence
  amino acid sequence produced on expression of the nucleic acids construct of Sequence ID No 21
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEPVDRVKQTLNFDLLKLAGDVESNPGPGNMALPVTALLLPLALLLHAARPGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL